(19) United States
(12) Patent Application Publication
(10) Pub. No.: US 2026/0108483 A1
(43) Pub. Date: Apr. 23, 2026

(54) METHODS AND COMPOSITIONS FOR PREVENTING THE PROGRESSION OF DIABETIC RETINOPATHY AND RELATED CONDITIONS

(71) Applicant: Opus Genetics, Inc., Durham, NC (US)

(72) Inventors: Mitchell George Brigell, Belmont, MA (US); Ronil Ajaykumar Patel, Tampa, FL (US); Jay Stuart Pepose, Chesterfield, MO (US); Mina Sooch, Bloomfield, MI (US)

(21) Appl. No.: 19/279,680

(22) Filed: Jul. 24, 2025

Related U.S. Application Data
(63) Continuation of application No. PCT/US2024/012879, filed on Jan. 25, 2024.
(60) Provisional application No. 63/441,054, filed on Jan. 25, 2023.

(51) Int. Cl. A61K 31/192; A61P 9/10; A61P 27/02

(52) U.S. Cl. CPC A61K 31/192; A61P 9/10; A61P 27/02

(57) ABSTRACT

The invention provides methods, compositions, and kits containing a first therapeutic agent that is a substituted 2,3-dimethoxyquinone of Formula I, or a pharmaceutically acceptable salt thereof, for preventing the progression of diabetic retinopathy, diabetic macular edema, and/or other retinal disorders and/or other disorders.

### CROSS-REFERENCE TO RELATED APPLICATIONS

This application is a continuation of International Patent Application No. PCT/US2024/012879, filed Jan. 25, 2024, which claims the benefit of and priority to U.S. Provisional Patent Application Ser. No. 63/441,054, filed Jan. 25, 2023; the contents of each of which are hereby incorporated by reference in their entirety.

### FIELD OF THE INVENTION

The invention provides methods, compositions, and kits containing a first therapeutic agent that is a substituted 2,3-dimethoxyquinone of Formula I, or a pharmaceutically acceptable salt thereof, for preventing the progression of diabetic retinopathy, diabetic macular edema, and/or other retinal disorders and/or other disorders.

### BACKGROUND

Diabetic retinopathy is a disease of the eye that, if left untreated, can lead to blindness. A significant proportion of individuals who suffer from diabetes experience some degree of related retinal damage. Existing therapies for diabetic retinopathy are not effective for all patients and/or have undesirable side effects. For example, laser photocoagulation produces its effects by creating burns in the tissue of the eye, which can be painful and/or cause certain vision problems (e.g., losses in peripheral, color, and/or night vision). Vitrectomy generally proceeds by creating an incision in the surface of the eye (introducing the potential for intraocular infection), and often requires weeks of recovery where the eye must be covered and cannot be used. Intravitreal injection of triamcinolone or anti-VEGF medications also carry a risk of intraocular infection, particularly with the need for additional injections over time.

The compound (E)-2-((4,5-dimethoxy-2-methyl-3,6-dioxocyclohexa-1,4-dien-1-yl)methylene)undecanoic acid, which has the following chemical formula, is described in WO 2009/042542 and WO 2022/232597:

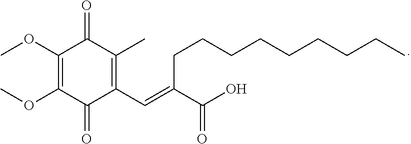 [SMILES: CCCCCCCCC/C(=C\C1=C(C)C(=O)C(OC)=C(OC)C1=O)C(=O)O]

Procedures for preventing the progression of diabetic retinopathy, diabetic macular edema, and/or other retinal disorders and/or other disorders using the foregoing compound would benefit patients.

The present invention addresses this need and provides other related advantages.

### SUMMARY

The invention provides methods, compositions, and kits containing a first therapeutic agent that is a substituted 2,3-dimethoxyquinone of Formula I, or a pharmaceutically acceptable salt thereof, for preventing the progression of diabetic retinopathy, diabetic macular edema, and/or other retinal disorders and/or other disorders. The methods generally comprise orally administering to a human patient in need thereof a therapeutically effective amount (e.g., an amount of from about 120 mg to about 600 mg per day) of a compound of Formula I or a pharmaceutically acceptable salt thereof:

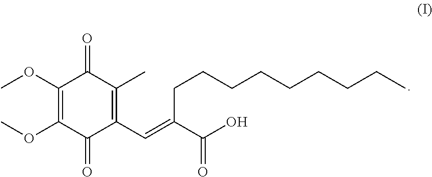 [SMILES: CCCCCCCCC/C(=C\C1=C(C)C(=O)C(OC)=C(OC)C1=O)C(=O)O]

Exemplary more preferred embodiments comprise orally administering to a human patient in need thereof an amount of from about 480 mg to about 600 mg per day of a compound of Formula I or pharmaceutically acceptable salt thereof. Preventing the progression of the patient's diabetic retinal disorder can be evaluated according to changes in the patient's Diabetic Retinopathy Severity Score (DRSS), the patient's visual acuity, and other procedures described in the literature. Additional exemplary aspects and embodiments of the invention are described below.

One aspect of the invention provides a method of preventing the progression of a diabetic retinal disease, comprising the steps of:
(a) identifying a human patient with diabetic retinal disease that would benefit from therapy that prevents the progression of the diabetic retinal disease; and
(b) orally administering to said human patient in need thereof a therapeutically effective amount of a first therapeutic agent to thereby prevent the progression of diabetic retinal disease, wherein the first therapeutic agent is a compound of Formula I or a pharmaceutically acceptable salt thereof:

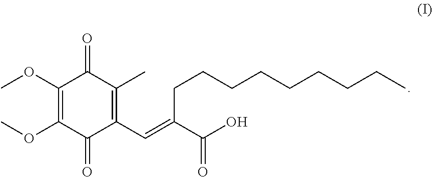 [SMILES: CCCCCCCCC/C(=C\C1=C(C)C(=O)C(OC)=C(OC)C1=O)C(=O)O]

In certain embodiments, a first dose of the first therapeutic agent and a second dose of the first therapeutic agent are orally administered to the patient on the same day. In certain embodiments, the method further comprises administering to the patient a second therapeutic agent that is a vascular endothelial growth factor inhibitor. In certain embodiments, the diabetic retinal disease is diabetic retinopathy. In certain embodiments, the diabetic retinal disease is diabetic macular edema. Additional features of the method are described in the detailed description.

Another aspect of the invention provides a method of reducing the rate of progression of a diabetic retinal disease, comprising the steps of:
(a) identifying a human patient with diabetic retinal disease that would benefit from therapy that reduces the rate of progression of the diabetic retinal disease; and
(b) orally administering to said human patient in need thereof a therapeutically effective amount of a first therapeutic agent to thereby reduce the rate of progression of the diabetic retinal disease, wherein the first therapeutic agent is a compound of Formula I or a pharmaceutically acceptable salt thereof:

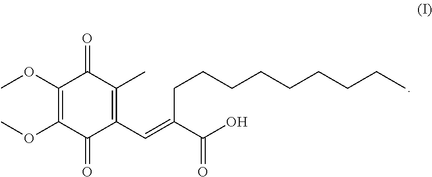 [SMILES: CCCCCCCCC/C(=C\C1=C(C)C(=O)C(OC)=C(OC)C1=O)C(=O)O]

In certain embodiments, a first dose of the first therapeutic agent and a second dose of the first therapeutic agent are orally administered to the patient on the same day. In certain embodiments, the diabetic retinal disease is diabetic retinopathy. Additional features of the method are described in the detailed description.

Another aspect of the invention provides a method of slowing clinically meaningful progression of a diabetic retinal disease, comprising the steps of:
(a) identifying a human patient with diabetic retinal disease that would benefit from therapy that slows clinically meaningful progression of the diabetic retinal disease; and
(b) orally administering to said human patient in need thereof a therapeutically effective amount of a first therapeutic agent to thereby slow clinically meaningful progression of the diabetic retinal disease, wherein the first therapeutic agent is a compound of Formula I or a pharmaceutically acceptable salt thereof:

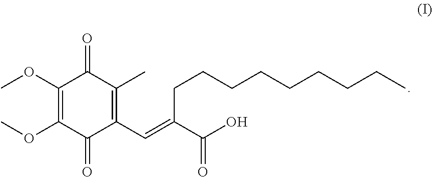 [SMILES: CCCCCCCCC/C(=C\C1=C(C)C(=O)C(OC)=C(OC)C1=O)C(=O)O]

In certain embodiments, a first dose of the first therapeutic agent and a second dose of the first therapeutic agent are orally administered to the patient on the same day. In certain embodiments, the diabetic retinal disease is diabetic retinopathy. Additional features of the method are described in the detailed description.

Another aspect of the invention provides a method of slowing the development of a vision-threatening complication in a patient suffering from a diabetic retinal disease, comprising the steps of:
(a) identifying a human patient with diabetic retinal disease that would benefit from therapy that slows the development of a vision-threatening complication; and
(b) orally administering to said human patient in need thereof a therapeutically effective amount of a first therapeutic agent to thereby slow the development of a vision-threatening complication, wherein the first therapeutic agent is a compound of Formula I or a pharmaceutically acceptable salt thereof:

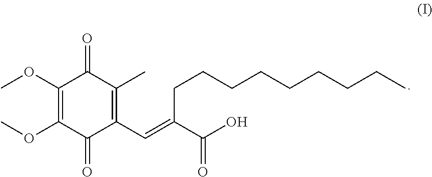 [SMILES: CCCCCCCCC/C(=C\C1=C(C)C(=O)C(OC)=C(OC)C1=O)C(=O)O]

In certain embodiments, the vision threatening complication is one or more of diabetic macular edema, proliferative diabetic retinopathy, vitreous hemorrhage, tractional retinal detachment, or anterior segment neovascularization. In certain embodiments, a first dose of the first therapeutic agent and a second dose of the first therapeutic agent are orally administered to the patient on the same day. In certain embodiments, the diabetic retinal disease is diabetic retinopathy. Additional features of the method are described in the detailed description.

Another aspect of the invention provides a method of preserving best-corrected visual acuity and/or reducing the need for a treatment selected from the group consisting of vascular endothelial growth factor inhibitor therapy and ocular laser therapy in a patient suffering from a diabetic retinal disease, comprising the steps of:
(a) identifying a human patient with diabetic retinal disease that would benefit from preserving best-corrected visual acuity and/or reducing the need for a treatment selected from the group consisting of vascular endothelial growth factor inhibitor therapy and ocular laser therapy; and
(b) orally administering to said human patient in need thereof a therapeutically effective amount of a first therapeutic agent, wherein the first therapeutic agent is a compound of Formula I or a pharmaceutically acceptable salt thereof:

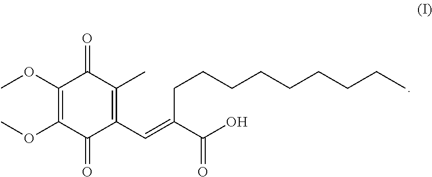 [SMILES: CCCCCCCCC/C(=C\C1=C(C)C(=O)C(OC)=C(OC)C1=O)C(=O)O]

In certain embodiments, a first dose of the first therapeutic agent and a second dose of the first therapeutic agent are orally administered to the patient on the same day. In certain embodiments, the diabetic retinal disease is diabetic retinopathy. Additional features of the method are described in the detailed description.

### BRIEF DESCRIPTION OF FIGURES

FIG. 1 depicts exemplary diabetic retinopathy severity scores (DRSS) and corresponding descriptions and retinal images.

FIG. 2 provides a more detailed description of exemplary diabetic retinopathy severity scores (DRSS) and retinal images.

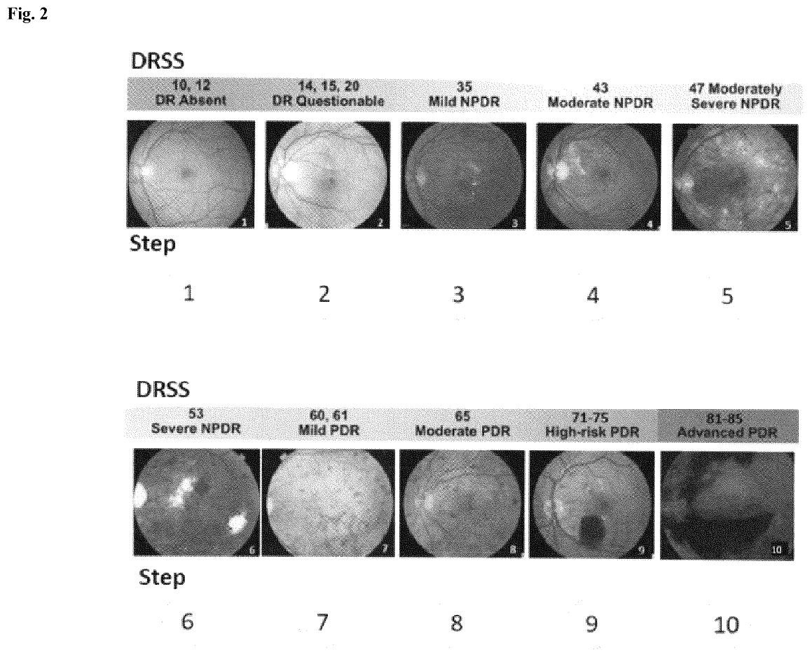

FIG. 3 shows results from a clinical trial, as further described in Example 1, that are the number of patients that experienced a three or more step worsening in the patients' binocular Diabetic Retinopathy Severity Scale Score at week 12 and at week 24 for patients that received placebo and for patients that received Compound 1.

FIG. 4A shows an XRPD pattern of a Compound 1 calcium salt obtained as described in Example 3.

FIG. 4B shows an overlay of a thermo-gravimetric (TG) thermogram and a differential scanning calorimetry (DSC) thermogram of the Compound 1 calcium salt obtained as described in Example 3.

FIG. 4C shows a dynamic vapor sorption (DVS) spectrum of the Compound 1 calcium salt obtained as described in Example 3.

FIG. 5A shows an XRPD pattern of the Compound 1 calcium salt obtained as described in Example 4.

FIG. 5B shows a ¹H NMR spectrum of the Compound 1 calcium salt obtained as described in Example 4.

FIG. 5C shows a thermo-gravimetric (TG) thermogram of Compound 1 calcium salt obtained as described in Example 4.

FIG. 5D shows a DSC thermogram of the Compound 1 calcium salt obtained as described in Example 4.

FIG. 6A shows an XRPD pattern of a Compound 1 calcium salt obtained as described in Example 5.

FIG. 6B shows an overlay of a TG thermogram and a DSC thermogram of the anhydrous and predominantly amorphous Compound 1 calcium salt obtained as described in Example 5.

### DETAILED DESCRIPTION OF THE INVENTION

The invention provides methods, compositions, and kits containing a first therapeutic agent that is a substituted 2,3-dimethoxyquinone of Formula I, or a pharmaceutically acceptable salt thereof, for preventing the progression of diabetic retinopathy, diabetic macular edema, and/or other retinal disorders and/or other disorders:

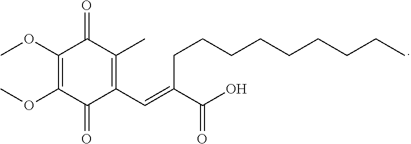 [SMILES: CCCCCCCCC/C(=C\C1=C(C)C(=O)C(OC)=C(OC)C1=O)C(=O)O]

Preventing the progression of the patient's diabetic retinal disorder can be evaluated according to changes in the patient's Diabetic Retinopathy Severity Score (DRSS), the patient's visual acuity, and other procedures described in the literature. Various aspects of the invention are set forth below in sections; however, aspects of the invention described in one particular section are not to be limited to any particular section.

### Definitions

To facilitate an understanding of the present invention, a number of terms and phrases are defined below.

The terms “a,” “an” and “the” as used herein mean “one or more” and include the plural unless the context is inappropriate.

The term “about” means within 10% of the stated value. In certain embodiments, the value may be within 8%, 6%, 5%, 4%, 2%, or 1% of the stated value.

As used herein, the term “patient” refers to organisms to be treated by the methods of the present invention. Such organisms preferably include, but are not limited to, mammals (e.g., murines, simians, equines, bovines, porcines, canines, felines, and the like), and most preferably includes humans.

As used herein, the term “effective amount” refers to the amount of a compound sufficient to effect beneficial or desired results. Unless specified otherwise, an effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin in Remington's Pharmaceutical Sciences, 15th Ed., Mack Publ. Co., Easton, PA [1975].

As used herein, the term “pharmaceutically acceptable salt” refers to any pharmaceutically acceptable salt (e.g., acid or base) of a compound of the present invention which, upon administration to a subject, is capable of providing a compound of this invention. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium or calcium), hydroxides, ammonia, and compounds of formula NW₃, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate (mesylate), 2-naphthalenesulfonate, nicotinate, oxalate, palmoate, pectinate, persulfate, phenylpropionate, picrate, pivalate, propionate, succinate, sulfate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. Therapeutic Methods

The invention provides methods for preventing the progression of diabetic retinopathy, diabetic macular edema, and/or other retinal disorders and/or other disorders by orally administering to a human patient a substituted 2,3-dimethoxyquinone of Formula I, or a pharmaceutically acceptable salt thereof. Preventing encompasses slowing the progression of the stated disease. Various aspects and embodiments of the therapeutic methods are described in the sections below. The sections are arranged for convenience and information in one section is not to be limited to that section, but may be applied to methods in other sections.

### A. First Method

One aspect of the invention provides a method of preventing the progression of a diabetic retinal disease, comprising the steps of:
(a) identifying a human patient with diabetic retinal disease that would benefit from therapy that prevents the progression of the diabetic retinal disease; and
(b) orally administering to said human patient in need thereof a therapeutically effective amount of a first therapeutic agent to thereby prevent the progression of diabetic retinal disease, wherein the first therapeutic agent is a compound of Formula I or a pharmaceutically acceptable salt thereof:

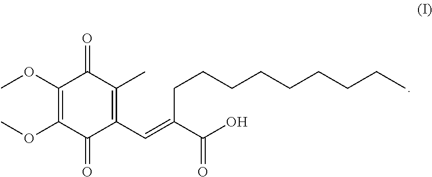 [SMILES: CCCCCCCCC/C(=C\C1=C(C)C(=O)C(OC)=C(OC)C1=O)C(=O)O]

The method may be further characterized by additional features, such as the identity of the first therapeutic agent and the dosing regimen. The invention embraces all permutations and combinations of these features.

Accordingly, the method may be further characterized according to the identity of the first therapeutic agent. For example, in certain embodiments, the first therapeutic agent is a compound of Formula I. In certain embodiments, the first therapeutic agent is a pharmaceutically acceptable salt of the compound of Formula I.

The method may be further characterized by features of step (a). For example, in certain embodiments, step (a) comprises identifying a human patient with diabetic retinal disease that is mild or moderate non-proliferative diabetic retinopathy that would benefit from therapy that prevents progression of the diabetic retinal disease. In certain embodiments, step (a) comprises identifying a human patient with diabetic retinal disease that is moderate to moderately severe non-proliferative diabetic retinopathy that would benefit from therapy that prevents progression of the diabetic retinal disease. In certain embodiments, step (a) comprises identifying a human patient with diabetic retinal disease that is mild proliferative diabetic retinopathy that would benefit from therapy that prevents progression of the diabetic retinal disease. In certain embodiments, step (a) comprises identifying a human patient with diabetic retinal disease that has not progressed beyond mild proliferative diabetic retinopathy that would benefit from therapy that prevents progression of the diabetic retinal disease. In certain embodiments, step (a) comprises identifying a human patient with diabetic retinal disease that is moderate to severe non-proliferative diabetic retinopathy that would benefit from therapy that prevents progression of the diabetic retinal disease.

In certain embodiments, preventing the progression of diabetic retinal disease comprises preventing an increase of three or more steps in the patient's Diabetic Retinopathy Severity Score. In certain embodiments, preventing the progression of diabetic retinal disease comprises preventing an increase of three steps in the patient's Diabetic Retinopathy Severity Score. In certain embodiments, preventing the progression of diabetic retinal disease comprises preventing an increase of two or more steps in the patient's Diabetic Retinopathy Severity Score. In certain embodiments, preventing the progression of diabetic retinal disease comprises preventing an increase of one or more steps in the patient's Diabetic Retinopathy Severity Score.

In certain embodiments, the patient's Diabetic Retinopathy Severity Score is the patient's binocular Diabetic Retinopathy Severity Score.

In certain embodiments, the increase in the patient's Diabetic Retinopathy Severity Score is over a period of 3 months. In certain embodiments, the increase in the patient's Diabetic Retinopathy Severity Score is over a period of 6 months. In certain embodiments, the increase in the patient's Diabetic Retinopathy Severity Score is over a period of 12 months.

### B. Second Method

Another aspect of the invention provides a method of preventing the progression of a diabetic retinal disease, comprising orally administering to said human patient in need thereof a therapeutically effective amount of a first therapeutic agent to thereby prevent the progression of diabetic retinal disease, wherein the first therapeutic agent is a compound of Formula I or a pharmaceutically acceptable salt thereof:

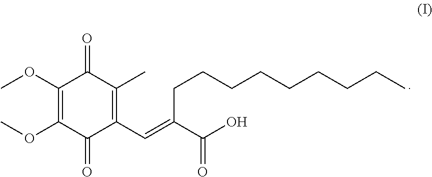 [SMILES: CCCCCCCCC/C(=C\C1=C(C)C(=O)C(OC)=C(OC)C1=O)C(=O)O]

The method may be further characterized by additional features, such as the identity of the first therapeutic agent and the dosing regimen. The invention embraces all permutations and combinations of these features.

Accordingly, the method may be further characterized according to the identity of the first therapeutic agent. For example, in certain embodiments, the first therapeutic agent is a compound of Formula I. In certain embodiments, the first therapeutic agent is a pharmaceutically acceptable salt of the compound of Formula I.

In certain embodiments, the method further comprises identifying a human patient with diabetic retinal disease that would benefit from therapy that prevents the progression of the diabetic retinal disease. In certain embodiments, the patient that would benefit from such therapy is a patient having a mild or moderate case of a diabetic retinal disease. In certain embodiments, the patient that would benefit from such therapy is a patient having a diabetic retinal disease that has not yet advanced to severe diabetic retinal disease.

In certain embodiments, method further comprises identifying a human patient with diabetic retinal disease that is mild or moderate non-proliferative diabetic retinopathy that would benefit from therapy that prevents progression of the diabetic retinal disease. In certain embodiments, method further comprises identifying a human patient with diabetic retinal disease that is moderate to moderately severe non-proliferative diabetic retinopathy that would benefit from therapy that prevents progression of the diabetic retinal disease. In certain embodiments, method further comprises identifying a human patient with diabetic retinal disease that is moderate to severe non-proliferative diabetic retinopathy that would benefit from therapy that prevents progression of the diabetic retinal disease. In certain embodiments, method further comprises identifying a human patient with diabetic retinal disease that is mild proliferative diabetic retinopathy that would benefit from therapy that prevents progression of the diabetic retinal disease. In certain embodiments, method further comprises identifying a human patient with diabetic retinal disease that has not progressed beyond mild proliferative diabetic retinopathy that would benefit from therapy that prevents progression of the diabetic retinal disease.

In certain embodiments, the diabetic retinal disease is diabetic retinopathy, and the patient has diabetic retinopathy that has not yet progressed to proliferative diabetic retinopathy. In certain embodiments, the diabetic retinal disease is diabetic retinopathy, and the patient has diabetic retinopathy that has not yet progressed beyond mild proliferative diabetic retinopathy.

In certain embodiments, the diabetic retinal disease is diabetic retinopathy, and the patient has non-proliferative diabetic retinopathy. In certain embodiments, the diabetic retinal disease is diabetic retinopathy, and the patient has mild non-proliferative diabetic retinopathy. In certain embodiments, the diabetic retinal disease is diabetic retinopathy, and the patient has moderate non-proliferative diabetic retinopathy. In certain embodiments, the diabetic retinal disease is diabetic retinopathy, and the patient has moderate to severe non-proliferative diabetic retinopathy. In certain embodiments, the diabetic retinal disease is diabetic retinopathy, and the patient has severe non-proliferative diabetic retinopathy.

In certain embodiments, preventing the progression of diabetic retinal disease comprises preventing an increase of three or more steps in the patient's Diabetic Retinopathy Severity Score. In certain embodiments, preventing the progression of diabetic retinal disease comprises preventing an increase of three steps in the patient's Diabetic Retinopathy Severity Score. In certain embodiments, preventing the progression of diabetic retinal disease comprises preventing an increase of two or more steps in the patient's Diabetic Retinopathy Severity Score. In certain embodiments, preventing the progression of diabetic retinal disease comprises preventing an increase of one or more steps in the patient's Diabetic Retinopathy Severity Score.

In certain embodiments, the patient's Diabetic Retinopathy Severity Score is the patient's binocular Diabetic Retinopathy Severity Score.

In certain embodiments, the increase in the patient's Diabetic Retinopathy Severity Score is over a period of 3 months. In certain embodiments, the increase in the patient's Diabetic Retinopathy Severity Score is over a period of 6 months. In certain embodiments, the increase in the patient's Diabetic Retinopathy Severity Score is over a period of 12 months.

In a more specific embodiment, the invention provides a method of preventing the progression of a diabetic retinal disease, comprising orally administering to said human patient in need thereof an amount of from about 720 mg to about 900 mg per day of a first therapeutic agent to thereby prevent the progression of diabetic retinal disease, wherein the first therapeutic agent is a compound of Formula I or a pharmaceutically acceptable salt thereof:

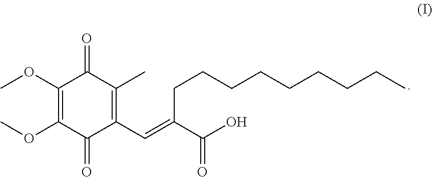 [SMILES: CCCCCCCCC/C(=C\C1=C(C)C(=O)C(OC)=C(OC)C1=O)C(=O)O]

### C. Third Method

Another aspect of the invention provides a method of reducing the rate of progression of a diabetic retinal disease, comprising the steps of.
(a) identifying a human patient with diabetic retinal disease that would benefit from therapy that reduces the rate of progression of the diabetic retinal disease; and
(b) orally administering to said human patient in need thereof a therapeutically effective amount of a first therapeutic agent to thereby reduce the rate of progression of the diabetic retinal disease, wherein the first therapeutic agent is a compound of Formula I or a pharmaceutically acceptable salt thereof.

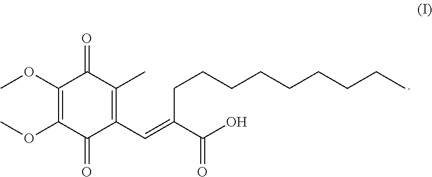 [SMILES: CCCCCCCCC/C(=C\C1=C(C)C(=O)C(OC)=C(OC)C1=O)C(=O)O]

The method may be further characterized by additional features, such as the identity of the first therapeutic agent and the dosing regimen. The invention embraces all permutations and combinations of these features.

Accordingly, the method may be further characterized according to the identity of the first therapeutic agent. For example, in certain embodiments, the first therapeutic agent is a compound of Formula I. In certain embodiments, the first therapeutic agent is a pharmaceutically acceptable salt of the compound of Formula I.

The method may be further characterized by features of step (a). For example, in certain embodiments, step (a) comprises identifying a human patient with diabetic retinal disease that is mild or moderate non-proliferative diabetic retinopathy that would benefit from therapy that reduces the rate of progression of the diabetic retinal disease. In certain embodiments, step (a) comprises identifying a human patient with diabetic retinal disease that is moderate to moderately severe non-proliferative diabetic retinopathy that would benefit from therapy that reduces the rate of progression of the diabetic retinal disease. In certain embodiments, step (a) comprises identifying a human patient with diabetic retinal disease that is mild proliferative diabetic retinopathy that would benefit from therapy that reduces the rate of progression of the diabetic retinal disease. In certain embodiments, step (a) comprises identifying a human patient with diabetic retinal disease that has not progressed beyond mild proliferative diabetic retinopathy that would benefit from therapy that reduces the rate of progression of the diabetic retinal disease. In certain embodiments, step (a) comprises identifying a human patient with diabetic retinal disease that is moderate to severe non-proliferative diabetic retinopathy that would benefit from therapy that prevents progression of the diabetic retinal disease.

In certain embodiments, reducing the rate of progression of diabetic retinal disease comprises increasing the amount of time that elapses before the patient experiences an increase of three or more steps in the patient's Diabetic Retinopathy Severity Score. In certain embodiments, reducing the rate of progression of diabetic retinal disease comprises increasing the amount of time that elapses before the patient experiences an increase of three steps in the patient's Diabetic Retinopathy Severity Score. In certain embodiments, reducing the rate of progression of diabetic retinal disease comprises increasing the amount of time that elapses before the patient experiences an increase of two or more steps in the patient's Diabetic Retinopathy Severity Score. In certain embodiments, reducing the rate of progression of diabetic retinal disease comprises increasing the amount of time that elapses before the patient experiences an increase of one or more steps in the patient's Diabetic Retinopathy Severity Score. In certain embodiments, the increase in time is at least a 10%, 20%, 30%, 40%, 50%, 60%, 70%, 80%, 90%, 100%, 150%, 200%, 250%, 300%, 350%, 400%, 450%, or 500% increase. In certain embodiments, the increase in time is an increase by at least 2, 4, 6, 8, 10, 12, 14, 16, or 18 months before the patient experiences the indicated increase in Diabetic Retinopathy Severity Score.

In certain embodiments, the patient's Diabetic Retinopathy Severity Score is the patient's binocular Diabetic Retinopathy Severity Score.

In certain embodiments, the increase in the patient's Diabetic Retinopathy Severity Score is over a period of 3 months. In certain embodiments, the increase in the patient's Diabetic Retinopathy Severity Score is over a period of 6 months. In certain embodiments, the increase in the patient's Diabetic Retinopathy Severity Score is over a period of 12 months.

### D. Fourth Method

Another aspect of the invention provides a method of reducing the rate of progression of a diabetic retinal disease, comprising orally administering to said human patient in need thereof a therapeutically effective amount of a first therapeutic agent to thereby reduce the rate of progression of the diabetic retinal disease, wherein the first therapeutic agent is a compound of Formula I or a pharmaceutically acceptable salt thereof.

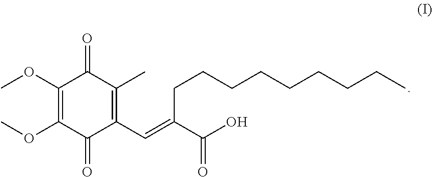 [SMILES: CCCCCCCCC/C(=C\C1=C(C)C(=O)C(OC)=C(OC)C1=O)C(=O)O]

The method may be further characterized by additional features, such as the identity of the first therapeutic agent and the dosing regimen. The invention embraces all permutations and combinations of these features.

Accordingly, the method may be further characterized according to the identity of the first therapeutic agent. For example, in certain embodiments, the first therapeutic agent is a compound of Formula I. In certain embodiments, the first therapeutic agent is a pharmaceutically acceptable salt of the compound of Formula I.

In certain embodiments, the method further comprises identifying a human patient with diabetic retinal disease that would benefit from therapy that reduces the rate of progression of the diabetic retinal disease. In certain embodiments, the patient that would benefit from such therapy is a patient having a mild or moderate case of a diabetic retinal disease. In certain embodiments, the patient that would benefit from such therapy is a patient having a diabetic retinal disease that has not yet advanced to severe diabetic retinal disease. In certain embodiments, the patient that would benefit from such therapy is a patient having a moderate to severe case of a diabetic retinal disease.

In certain embodiments, the method further comprises identifying a human patient with diabetic retinal disease that is mild or moderate non-proliferative diabetic retinopathy that would benefit from therapy that reduces the rate of progression of the diabetic retinal disease. In certain embodiments, the method further comprises identifying a human patient with diabetic retinal disease that is moderate to moderately severe non-proliferative diabetic retinopathy that would benefit from therapy that reduces the rate of progression of the diabetic retinal disease. In certain embodiments, the method further comprises identifying a human patient with diabetic retinal disease that is mild proliferative diabetic retinopathy that would benefit from therapy that reduces the rate of progression of the diabetic retinal disease. In certain embodiments, the method further comprises identifying a human patient with diabetic retinal disease that has not progressed beyond mild proliferative diabetic retinopathy that would benefit from therapy that reduces the rate of progression of the diabetic retinal disease.

In certain embodiments, the diabetic retinal disease is diabetic retinopathy, and the patient has diabetic retinopathy that has not yet progressed to proliferative diabetic retinopathy. In certain embodiments, the diabetic retinal disease is diabetic retinopathy, and the patient has diabetic retinopathy that has not yet progressed beyond mild proliferative diabetic retinopathy.

In certain embodiments, the diabetic retinal disease is diabetic retinopathy, and the patient has non-proliferative diabetic retinopathy. In certain embodiments, the diabetic retinal disease is diabetic retinopathy, and the patient has mild non-proliferative diabetic retinopathy. In certain embodiments, the diabetic retinal disease is diabetic retinopathy, and the patient has moderate non-proliferative diabetic retinopathy. In certain embodiments, the diabetic retinal disease is diabetic retinopathy, and the patient has moderate to severe non-proliferative diabetic retinopathy. In certain embodiments, the diabetic retinal disease is diabetic retinopathy, and the patient has severe non-proliferative diabetic retinopathy.

In certain embodiments, reducing the rate of progression of diabetic retinal disease comprises increasing the amount of time that elapses before the patient experiences an increase of three or more steps in the patient's Diabetic Retinopathy Severity Score. In certain embodiments, reducing the rate of progression of diabetic retinal disease comprises increasing the amount of time that elapses before the patient experiences an increase of three steps in the patient's Diabetic Retinopathy Severity Score. In certain embodiments, reducing the rate of progression of diabetic retinal disease comprises increasing the amount of time that elapses before the patient experiences an increase of two or more steps in the patient's Diabetic Retinopathy Severity Score. In certain embodiments, reducing the rate of progression of diabetic retinal disease comprises increasing the amount of time that elapses before the patient experiences an increase of one or more steps in the patient's Diabetic Retinopathy Severity Score. In certain embodiments, the increase in time is at least a 10%, 20%, 30%, 40%, 50%, 60%, 70%, 80%, 90%, 100%, 150%, 200%, 250%, 300%, 350%, 400%, 450%, or 500% increase. In certain embodiments, the increase in time is an increase by at least 2, 4, 6, 8, 10, 12, 14, 16, or 18 months before the patient experiences the indicated increase in Diabetic Retinopathy Severity Score.

In certain embodiments, the patient's Diabetic Retinopathy Severity Score is the patient's binocular Diabetic Retinopathy Severity Score.

In certain embodiments, the increase in the patient's Diabetic Retinopathy Severity Score is over a period of 3 months. In certain embodiments, the increase in the patient's Diabetic Retinopathy Severity Score is over a period of 6 months. In certain embodiments, the increase in the patient's Diabetic Retinopathy Severity Score is over a period of 12 months.

### E. Fifth Method

Another aspect of the invention provides a method of slowing clinically meaningful progression of a diabetic retinal disease, comprising orally administering to said human patient in need thereof a therapeutically effective amount of a first therapeutic agent to thereby slow clinically meaningful progression of the diabetic retinal disease, wherein the first therapeutic agent is a compound of Formula I or a pharmaceutically acceptable salt thereof:

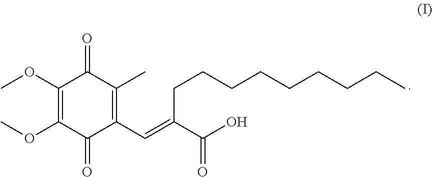 [SMILES: CCCCCCCCC/C(=C\C1=C(C)C(=O)C(OC)=C(OC)C1=O)C(=O)O]

Another aspect of the invention provides a method of slowing clinically meaningful progression of a diabetic retinal disease, comprising the steps of:
(a) identifying a human patient with diabetic retinal disease that would benefit from therapy that slows clinically meaningful progression of the diabetic retinal disease; and
(b) orally administering to said human patient in need thereof a therapeutically effective amount of a first therapeutic agent to thereby slow clinically meaningful progression of the diabetic retinal disease, wherein the first therapeutic agent is a compound of Formula I or a pharmaceutically acceptable salt thereof:

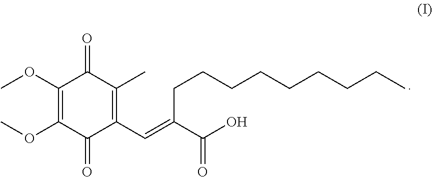 [SMILES: CCCCCCCCC/C(=C\C1=C(C)C(=O)C(OC)=C(OC)C1=O)C(=O)O]

The methods may be further characterized by additional features, such as the identity of the first therapeutic agent and the dosing regimen. The invention embraces all permutations and combinations of these features.

### F. Sixth Method

Another aspect of the invention provides a method of slowing the development of a vision-threatening complication in a patient suffering from a diabetic retinal disease, comprising orally administering to said human patient in need thereof a therapeutically effective amount of a first therapeutic agent to thereby slow the development of a vision-threatening complication, wherein the first therapeutic agent is a compound of Formula I or a pharmaceutically acceptable salt thereof:

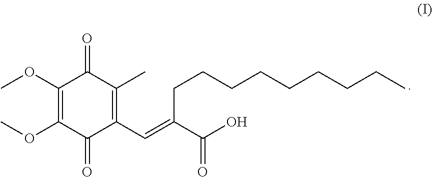 [SMILES: CCCCCCCCC/C(=C\C1=C(C)C(=O)C(OC)=C(OC)C1=O)C(=O)O]

Another aspect of the invention provides a method of slowing the development of a vision-threatening complication in a patient suffering from a diabetic retinal disease, comprising the steps of:
(a) identifying a human patient with diabetic retinal disease that would benefit from therapy that slows the development of a vision-threatening complication; and
(b) orally administering to said human patient in need thereof a therapeutically effective amount of a first therapeutic agent to thereby slow the development of a vision-threatening complication, wherein the first therapeutic agent is a compound of Formula I or a pharmaceutically acceptable salt thereof:

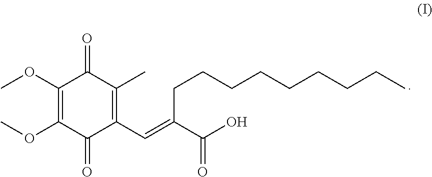 [SMILES: CCCCCCCCC/C(=C\C1=C(C)C(=O)C(OC)=C(OC)C1=O)C(=O)O]

In certain embodiments, the vision threatening complication is one or more of diabetic macular edema, proliferative diabetic retinopathy, vitreous hemorrhage, tractional retinal detachment, or anterior segment neovascularization.

The methods may be further characterized by additional features, such as the identity of the first therapeutic agent and the dosing regimen. The invention embraces all permutations and combinations of these features.

### G. Seventh Method

Another aspect of the invention provides a method of preserving best-corrected visual acuity and/or reducing the need for a treatment selected from the group consisting of vascular endothelial growth factor inhibitor therapy and ocular laser therapy in a patient suffering from a diabetic retinal disease, comprising orally administering to said human patient in need thereof a therapeutically effective amount of a first therapeutic agent, wherein the first therapeutic agent is a compound of Formula I or a pharmaceutically acceptable salt thereof:

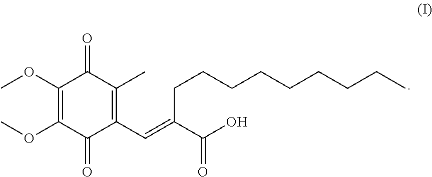 [SMILES: CCCCCCCCC/C(=C\C1=C(C)C(=O)C(OC)=C(OC)C1=O)C(=O)O]

Another aspect of the invention provides a method of preserving best-corrected visual acuity and/or reducing the need for a treatment selected from the group consisting of vascular endothelial growth factor inhibitor therapy and ocular laser therapy in a patient suffering from a diabetic retinal disease, comprising the steps of.
(a) identifying a human patient with diabetic retinal disease that would benefit from preserving best-corrected visual acuity and/or reducing the need for a treatment selected from the group consisting of vascular endothelial growth factor inhibitor therapy and ocular laser therapy; and
(b) orally administering to said human patient in need thereof a therapeutically effective amount of a first therapeutic agent, wherein the first therapeutic agent is a compound of Formula I or a pharmaceutically acceptable salt thereof.

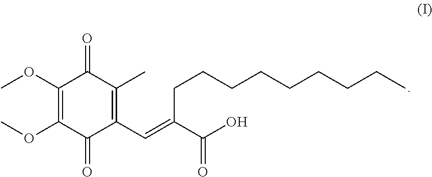 [SMILES: CCCCCCCCC/C(=C\C1=C(C)C(=O)C(OC)=C(OC)C1=O)C(=O)O]

The methods may be further characterized by additional features, such as the identity of the first therapeutic agent and the dosing regimen. The invention embraces all permutations and combinations of these features.

H. Additional Features for the Fifth through Seventh Therapeutic Methods

Additional features that may be used to characterize the Fifth through Seventh Therapeutic Methods are provided below.

### Identity of the Diabetic Retinal Disease

In certain embodiments, the diabetic retinal disease is diabetic retinopathy. In certain embodiments, the diabetic retinopathy is moderate diabetic retinopathy. In certain embodiments, the diabetic retinopathy is moderately severe to severe diabetic retinopathy. In certain embodiments, the diabetic retinopathy is moderate to severe diabetic retinopathy. In certain embodiments, the diabetic retinopathy is non-proliferative diabetic retinopathy. In a more specific embodiment, the diabetic retinopathy is moderate to severe non-proliferative diabetic retinopathy.

### Step (a) More Detailed Embodiments

In certain embodiments, step (a) comprises identifying a human patient with diabetic retinal disease that is mild or moderate non-proliferative diabetic retinopathy that would benefit from therapy. In certain embodiments, step (a) comprises identifying a human patient with diabetic retinal disease that is moderate to moderately severe non-proliferative diabetic retinopathy that would benefit from therapy. In certain embodiments, step (a) comprises identifying a human patient with diabetic retinal disease that is moderate to severe non-proliferative diabetic retinopathy that would benefit from therapy. In certain embodiments, step (a) comprises identifying a human patient with diabetic retinal disease that is moderate to severe non-proliferative diabetic retinopathy in both eyes that would benefit from therapy, and wherein at least one eye of the patient has a DRSS level of 47 or 53. In certain embodiments, step (a) comprises identifying a human patient with diabetic retinal disease that has not progressed beyond mild proliferative diabetic retinopathy that would benefit from therapy.

### First Therapeutic Agent & Dose Thereof

In certain embodiments, the first therapeutic agent is a compound of Formula I. In certain embodiments, the first therapeutic agent is a pharmaceutically acceptable salt of the compound of Formula I. In certain embodiments, the first therapeutic agent is a calcium salt of the compound of Formula I.

In certain embodiments, the first therapeutic agent is orally administered to the patient in an amount of from about 300 mg to about 600 mg per day. In certain embodiments, a first dose of the first therapeutic agent and a second dose of the first therapeutic agent are orally administered to the patient on the same day. In certain embodiments, the first therapeutic agent is orally administered to the patient in an amount of about 600 mg per day. In certain embodiments, about 300 mg of the first therapeutic agent is orally administered to the patient in the morning, and about 300 mg of the first therapeutic agent is orally administered to the patient in the evening. In certain embodiments, about 300 mg of the first therapeutic agent is orally administered to the patient, and then at a time that is from about 8 hours to about 16 hours later about 300 mg of the first therapeutic agent is orally administered to the patient.

In certain embodiments, if the patient experiences an adverse event due to the first therapeutic agent, then thereafter for a period of at least two days the first therapeutic agent is orally administered to the patient in the reduced-daily amount of from about 300 mg to about 480 mg per day. In certain embodiments, if the patient experiences an adverse event due to the first therapeutic agent, then thereafter for a period of at least two days the first therapeutic agent is orally administered to the patient in the reduced-daily amount of about 300 mg per day.

In certain embodiments, the first therapeutic agent is orally administered to a patient in an amount of about 480 mg per day. In certain embodiments, the first therapeutic agent is orally administered to the patient in an amount of about 300 mg per day.

In certain embodiments, the first therapeutic agent is orally administered to the patient in the morning.

In certain embodiments, the first therapeutic agent is orally administered to a patient only 1 time per day.

In certain embodiments, the first therapeutic agent is orally administered to the patient daily for at least 12 weeks. In certain embodiments, the first therapeutic agent is orally administered to the patient daily for at least 24 weeks. In certain embodiments, the first therapeutic agent is orally administered to the patient daily for at least 36 weeks. In certain embodiments, the first therapeutic agent is orally administered to the patient daily for at least 48 weeks. In certain embodiments, the first therapeutic agent is orally administered to the patient daily for at least 52 weeks. In certain embodiments, the first therapeutic agent is orally administered to the patient daily for at least 96 weeks.

### Identity of the Patient

In certain embodiments, the human patient is an adult human patient. In certain embodiments, the patient has a DRSS level of 47 or 53 in at least one eye.

### Exemplary Additional Performance Features of the Methods

In certain embodiments, any increase in blood plasma concentration of alanine aminotransferase due to the first therapeutic agent is no greater than 50%. In certain embodiments, any increase in blood plasma concentration of aspartate aminotransferase due to the first therapeutic agent is no greater than 50%. In certain embodiments, any reduction in glomerular filtration rate in the patient is no greater than 25%.

In certain embodiments, the incidence of any eye disorder due to the first therapeutic agent occurs no more frequently than one patient for every ten patients subjected to the same treatment. In certain embodiments, the incidence of any eye disorder due to the first therapeutic agent occurs no more frequently than one patient for every twenty patients subjected to the same treatment. In certain embodiments, the incidence of any gastrointestinal disorder due to the first therapeutic agent occurs no more frequently than one patient for every ten patients subjected to the same treatment.

In certain embodiments, the incidence of any nervous system disorder due to the first therapeutic agent occurs no more frequently than one patient for every twenty patients subjected to the same treatment.

In certain embodiments, the method is further characterized by achieving a reduction in blood plasma concentration of alanine aminotransferase due to the first therapeutic agent.

In certain embodiments, the method is further characterized by achieving a reduction in blood plasma concentration of aspartate aminotransferase due to the first therapeutic agent.

In certain embodiments, the method is further characterized by preventing the loss of visual acuity in the subject. In certain embodiments, the method is further characterized by preventing the loss of visual acuity in the subject characterized by a change in visual acuity in the amount of five or more letters on a vision chart. In certain embodiments, the change in visual acuity is over 12 weeks, 24 weeks, 48 weeks, or 52 weeks.

### I. General Considerations for Therapeutic Methods

General considerations that may be applied to therapeutic methods described herein (e.g., the methods described in Parts A and D above, and Parts E through G above) are provided below and include, for example, the amount of first therapeutic agent administered, the duration of daily oral administration of the first therapeutic agent, characteristics of the disease or condition to be treated (e.g., characteristics of the diabetic retinal disease), and the identity of the human patient. A more thorough description of such features is provided below. The invention embraces all permutations and combinations of these features.

### Dosing Regimen of First Therapeutic Agent

The methods may be further characterized according to the dosing regimen. For example, in certain embodiments, a first dose of the first therapeutic agent and a second dose of the first therapeutic agent are orally administered to the patient on the same day. In certain embodiments, the first therapeutic agent is orally administered to a patient only 1 time per day.

In certain embodiments, the first therapeutic agent is orally administered to the patient in an amount of greater than 600 mg per day. In certain embodiments, the first therapeutic agent is orally administered to the patient in an amount of from about 720 mg to about 900 mg per day. In certain embodiments, about 360 mg of the first therapeutic agent is orally administered to the patient, and then at a time that is from about 8 hours to about 16 hours later about 360 mg of the first therapeutic agent is orally administered to the patient. In certain embodiments, about 360 mg of the first therapeutic agent is orally administered to the patient in the morning, and then about 360 mg of the first therapeutic agent is orally administered to the patient in the evening. In certain embodiments, about 480 mg of the first therapeutic agent is orally administered to the patient, and then at a time that is from about 8 hours to about 16 hours later about 240 mg of the first therapeutic agent is orally administered to the patient. In certain embodiments, about 480 mg of the first therapeutic agent is orally administered to the patient in the morning, and then about 240 mg of the first therapeutic agent is orally administered to the patient in the evening. In certain embodiments, about 240 mg of the first therapeutic agent is orally administered to the patient in the morning, and then about 480 mg of the first therapeutic agent is orally administered to the patient in the evening. In certain embodiments, about 600 mg of the first therapeutic agent is orally administered to the patient, and then at a time that is from about 8 hours to about 16 hours later about 300 mg of the first therapeutic agent is orally administered to the patient. In certain embodiments, about 300 mg of the first therapeutic agent is orally administered to the patient at three different times during the day. In certain embodiments, about 300 mg of the first therapeutic agent is orally administered to the patient, and then at a time that is from about 4 hour to about 8 hours later about 300 mg of the first therapeutic agent is orally administered to the patient, and then at a time that is from about 4 hour to about 8 hours later about 300 mg of the first therapeutic agent is orally administered to the patient. In certain embodiments, about 600 mg of the first therapeutic agent is orally administered to the patient in the morning, and then about 300 mg of the first therapeutic agent is orally administered to the patient in the evening. In certain embodiments, about 300 mg of the first therapeutic agent is orally administered to the patient in the morning, and then about 600 mg of the first therapeutic agent is orally administered to the patient in the evening.

In certain embodiments, the first therapeutic agent is orally administered to the patient in an amount of about 720 mg per day. In certain embodiments, the first therapeutic agent is orally administered to the patient in an amount of about 900 mg per day. In certain embodiments, the first therapeutic agent is orally administered to the patient in an amount of from about 480 mg to about 600 mg per day. In certain embodiments, the first therapeutic agent is orally administered to the patient in an amount of about 600 mg per day.

In certain embodiments, about 360 mg of the first therapeutic agent is orally administered to the patient in the morning, and about 240 mg of the first therapeutic agent is orally administered to the patient in the evening. In certain embodiments, about 360 mg of the first therapeutic agent is orally administered to the patient, and then at a time that is from about 8 hours to about 16 hours later about 240 mg of the first therapeutic agent is orally administered to the patient. In certain embodiments, about 360 mg of the first therapeutic agent is orally administered to the patient, and then at a time that is from about 10 hours to about 14 hours later about 240 mg of the first therapeutic agent is orally administered to the patient.

In certain embodiments, about 240 mg of the first therapeutic agent is orally administered to the patient in the morning, and about 360 mg of the first therapeutic agent is orally administered to the patient in the evening. In certain embodiments, about 240 mg of the first therapeutic agent is orally administered to the patient, and then at a time that is from about 8 hours to about 16 hours later about 360 mg of the first therapeutic agent is orally administered to the patient. In certain embodiments, about 240 mg of the first therapeutic agent is orally administered to the patient, and then at a time that is from about 10 hours to about 14 hours later about 360 mg of the first therapeutic agent is orally administered to the patient.

In certain embodiments, about 300 mg of the first therapeutic agent is orally administered to the patient in the morning, and about 300 mg of the first therapeutic agent is orally administered to the patient in the evening. In certain embodiments, about 300 mg of the first therapeutic agent is orally administered to the patient, and then at a time that is from about 8 hours to about 16 hours later about 300 mg of the first therapeutic agent is orally administered to the patient. In certain embodiments, about 300 mg of the first therapeutic agent is orally administered to the patient, and then at a time that is from about 10 hours to about 14 hours later about 300 mg of the first therapeutic agent is orally administered to the patient.

In certain embodiments, if the patient experiences an adverse event due to the first therapeutic agent, then thereafter for a period of at least two days the first therapeutic agent is orally administered to the patient in the reduced-daily amount of from about 300 mg to about 480 mg per day. In certain embodiments, if the patient experiences an adverse event due to the first therapeutic agent, then thereafter for a period of at least two days the first therapeutic agent is orally administered to the patient in the reduced-daily amount of about 480 mg per day.

In certain embodiments, if the patient experiences an adverse event due to the first therapeutic agent, then thereafter for a period of at least two days the first therapeutic agent is orally administered to the patient in the reduced-daily amount of from about 120 mg to about 300 mg per day.

In certain embodiments, the first therapeutic agent is orally administered to a patient in an amount of about 480 mg per day.

In certain embodiments, about 240 mg of the first therapeutic agent is orally administered to the patient in the morning, and about 240 mg of the first therapeutic agent is orally administered to the patient in the evening. In certain embodiments, about 240 mg of the first therapeutic agent is orally administered to the patient, and then at a time that is from about 8 hours to about 16 hours later about 240 mg of the first therapeutic agent is orally administered to the patient. In certain embodiments, about 240 mg of the first therapeutic agent is orally administered to the patient, and then at a time that is from about 10 hours to about 14 hours later about 240 mg of the first therapeutic agent is orally administered to the patient.

In certain embodiments, if the patient experiences an adverse event due to the first therapeutic agent, then thereafter for a period of at least two days the first therapeutic agent is orally administered to the patient in the reduced-daily amount of about 300 mg per day. In certain embodiments, the first therapeutic agent is orally administered to the patient in the morning.

In certain embodiments, the first therapeutic agent is orally administered to the patient in an amount of about 300 mg per day. In certain embodiments, the first therapeutic agent is orally administered to the patient in an amount of about 240 mg per day. In certain embodiments, the first therapeutic agent is orally administered to the patient in an amount of about 120 mg per day.

### Duration of Daily Administration of the First Therapeutic Agent

The methods may be further characterized according to the duration of daily oral administration of the first therapeutic agent.

In certain embodiments, the first therapeutic agent is orally administered to the patient daily for at least 1 week. In certain embodiments, the first therapeutic agent is orally administered to the patient daily for at least 2 weeks. In certain embodiments, the first therapeutic agent is orally administered to the patient daily for at least 4 weeks. In certain embodiments, the first therapeutic agent is orally administered to the patient daily for at least 6 weeks. In certain embodiments, the first therapeutic agent is orally administered to the patient daily for at least 8 weeks. In certain embodiments, the first therapeutic agent is orally administered to the patient daily for at least 10 weeks. In certain embodiments, the first therapeutic agent is orally administered to the patient daily for at least 12 weeks. In certain embodiments, of the first therapeutic agent is orally administered to the patient daily for at least 24 weeks. In certain embodiments, of the first therapeutic agent is orally administered to the patient daily for at least 36 weeks. In certain embodiments, of the first therapeutic agent is orally administered to the patient daily for at least 48 weeks. In certain embodiments, of the first therapeutic agent is orally administered to the patient daily for at least 52 weeks. In certain embodiments, of the first therapeutic agent is orally administered to the patient daily for at least 96 weeks. In certain embodiments, the first therapeutic agent is orally administered to the patient daily for at least 30, 32, 34, 36, 38 40, 42, 44, 46, 48, 50, or 52 weeks.

In certain embodiments, the amount of the first therapeutic agent is orally administered to the patient daily for at least 1 week. In certain embodiments, the amount of the first therapeutic agent is orally administered to the patient daily for at least 2 weeks. In certain embodiments, the amount of the first therapeutic agent is orally administered to the patient daily for at least 4 weeks. In certain embodiments, the amount of the first therapeutic agent is orally administered to the patient daily for at least 6 weeks. In certain embodiments, the amount of the first therapeutic agent is orally administered to the patient daily for at least 8 weeks. In certain embodiments, the amount of the first therapeutic agent is orally administered to the patient daily for at least 10 weeks. In certain embodiments, the amount of the first therapeutic agent is orally administered to the patient daily for at least 12 weeks. In certain embodiments, the amount of the first therapeutic agent is orally administered to the patient daily for at least 24 weeks. In certain embodiments, the amount of the first therapeutic agent is orally administered to the patient daily for at least 48 weeks. In certain embodiments, the amount of the first therapeutic agent is orally administered to the patient daily for at least 52 weeks. In certain embodiments, the amount of the first therapeutic agent is orally administered to the patient daily for at least 96 weeks. In certain embodiments, the amount of the first therapeutic agent is orally administered to the patient daily for at least 30, 32, 34, 36, 38 40, 42, 44, 46, 48, 50, or 52 weeks.

### Characteristics of the Diabetic Retinal Disease

The methods may be further characterized according to characteristics of the diabetic retinal disease. For example, in certain embodiments, the diabetic retinal disease is diabetic retinopathy. In certain embodiments, the diabetic retinopathy is mild diabetic retinopathy. In certain embodiments, the diabetic retinopathy is moderate diabetic retinopathy. In certain embodiments, the diabetic retinopathy is moderately severe to severe diabetic retinopathy. In certain embodiments, the diabetic retinopathy is moderate to severe diabetic retinopathy. In certain embodiments, the diabetic retinopathy is non-proliferative diabetic retinopathy. In certain embodiments, the diabetic retinopathy is proliferative diabetic retinopathy.

In certain embodiments, the diabetic retinal disease is diabetic macular edema. In certain embodiments, the diabetic retinopathy is proliferative diabetic retinopathy or diabetic macular edema.

### Additional Considerations

The methods may be further characterized according to additional considerations, such as the form in which the first therapeutic agent is administered, identity of the human patient, and improvement in diabetic retinal disease achieved by the method.

For example, in certain embodiments, the first therapeutic agent is orally administered to the patient in the form of an extended-release pharmaceutical composition. in certain embodiments, the first therapeutic agent is orally administered to the patient in the form of an extended-release pharmaceutical composition that provides release of the first therapeutic agent for duration of at least 1, 2, 3, 4, 5, 6, 7, 8, 9, 10, 11, or 12 hours. In certain embodiments, the first therapeutic agent is orally administered to the patient in the form of an immediate-release pharmaceutical composition. Pharmaceutical compositions are described in further detail in Section III below.

In certain embodiments, the human patient is an adult human patient. In certain embodiments, the patient has a DRSS level of 47 or 53 in at least one eye.

In certain embodiments, the method is further characterized according to the improvement in best-corrected visual acuity. For example, in certain embodiments, the patient experiences an improvement of at least 5%, 10%, 15%, 20%, 25%, 30%, 40%, 50%, 60%, 70%, 80%, 90% or 100% in best-corrected visual acuity due to the method. In certain embodiments, the patient experiences an improvement of at least 2, 4, 6, 8, 10, 12, 14, 16, or 18 letters in best-corrected visual acuity due to the method. Best-corrected visual acuity can be measured according to methods known in the art, for example, with a Standard ETDRS illuminated chart (on wall or stand) at 4 m. Alternatively, best-corrected visual acuity can be measured using a Snellen chart.

In certain embodiments, the method is further characterized according to the preventing the loss of visual acuity in the subject. In certain embodiments, the method is further characterized by preventing the loss of visual acuity in the subject characterized by a change in visual acuity in the amount of five or more letters on a vision chart. In certain embodiments, the vision chart is a Snellen chart. In certain embodiments, the change in visual acuity is over 12 weeks or 24 weeks. In certain embodiments, the change in visual acuity is over 12 weeks. In certain embodiments, the change in visual acuity is over 24 weeks. In certain embodiments, the change in visual acuity is over 48 weeks.

In certain embodiments, the method is further characterized according to impact on a symptom of diabetes. In certain embodiments, the method reduces a symptom of diabetes. In certain embodiments, the method reduces any renal impairment experienced by the patient. In certain embodiments, the method reduces any renal impairment experienced by the patient by at least 5, 10, 12, 14, 16, 18, 20, 22, 24, 26, 28, 30, 32, 34, 36, 38, 40, 50, 60, 70, 80, or 90 percent. The said reduction in renal impairment is relative to that experienced by a comparable patient that has not received therapy according to the method using the first therapeutic agent.

In certain embodiments, the method achieves a neuroprotective effect.

In certain embodiments, the method is further characterized by the feature that any increase in blood plasma concentration of alanine aminotransferase due to the first therapeutic agent is no greater than 50%. In certain embodiments, the method is further characterized by the feature that any increase in blood plasma concentration of alanine aminotransferase due to the first therapeutic agent is no greater than 2, 3, 4, 5, 6, 7, 8, 9, 10, 11, 12, 13, 14, 15, 16, 17, 18, 19, or 20 percent. In certain embodiments, the method is further characterized by the feature that any increase in blood plasma concentration of alanine aminotransferase due to the first therapeutic agent is no greater than 30, 40, 50, 60, 70, 80 or 90 percent.

In certain embodiments, the method is further characterized by the feature it results in a reduction in blood plasma concentration of alanine aminotransferase due to the first therapeutic agent. In certain embodiments, the reduction is at least 2, 3, 4, 5, 6, 7, 8, 9, 10, 11, 12, 13, 14, 15, 16, 17, 18, 19, or 15 percent. In certain embodiments, the reduction is at least 10 percent.

In certain embodiments, the method is further characterized by the feature that any increase in blood plasma concentration of aspartate aminotransferase due to the first therapeutic agent is no greater than 50%. In certain embodiments, the method is further characterized by the feature that any increase in blood plasma concentration of aspartate aminotransferase due to the first therapeutic agent is no greater than 2, 3, 4, 5, 6, 7, 8, 9, 10, 11, 12, 13, 14, 15, 16, 17, 18, 19, or 25 percent. In certain embodiments, the method is further characterized by the feature that any increase in blood plasma concentration of aspartate aminotransferase due to the first therapeutic agent is no greater than 30, 40, 50, 60, 70, 80 or 90 percent.

In certain embodiments, the method is further characterized by the feature it results in a reduction in blood plasma concentration of aspartate aminotransferase due to the first therapeutic agent. In certain embodiments, the reduction is at least 2, 3, 4, 5, 6, 7, 8, 9, 10, 11, 12, 13, 14, 15, 16, 17, 18, 19, or 15 percent. In certain embodiments, the reduction is at least 10 percent.

In certain embodiments, the method is further characterized by the feature that any reduction in glomerular filtration rate in the patient is no greater than 15%. In certain embodiments, the method is further characterized by the feature that any reduction in glomerular filtration rate in the patient is no greater than 2, 3, 4, 5, 6, 7, 8, 9, 10, 11, 12, 13, 14, 15, 16, 17, 18, 19, 20, 21, 22, 23, 24, or 25 percent. In certain embodiments, the method is further characterized by the feature that any reduction in glomerular filtration rate in the patient is no greater than 25 percent.

In certain embodiments, the method is further characterized by the feature that the incidence of any eye disorder due to the first therapeutic agent occurs no more frequently than one patient for every ten patients subjected to the same treatment. In certain embodiments, the method is further characterized by the feature that the incidence of any eye disorder due to the first therapeutic agent occurs no more frequently than one patient for every 10, 12, 14, 16, 18, 20, 22, 24, 26, 28, 30, 32, 34, 36, 38, or 40 patients subjected to the same treatment.

In certain embodiments, the method is further characterized by the feature that the incidence of any eye disorder due to the first therapeutic agent occurs no more frequently than one patient for every twenty patients subjected to the same treatment. In certain embodiments, the method is further characterized by the feature that the incidence of any eye disorder due to the first therapeutic agent occurs no more frequently than one patient for every 10, 12, 14, 16, 18, 20, 22, 24, 26, 28, 30, 32, 34, 36, 38, or 40 patients subjected to the same treatment.

In certain embodiments, the method is further characterized by the feature that the incidence of any gastrointestinal disorder due to the first therapeutic agent occurs no more frequently than one patient for every ten patients subjected to the same treatment. In certain embodiments, the method is further characterized by the feature that the incidence of any gastrointestinal disorder due to the first therapeutic agent occurs no more frequently than one patient for every 10, 12, 14, 16, 18, 20, 22, 24, 26, 28, 30, 32, 34, 36, 38, or 40 patients subjected to the same treatment.

In certain embodiments, the method is further characterized by the feature that the incidence of any nervous system disorder due to the first therapeutic agent occurs no more frequently than one patient for every twenty patients subjected to the same treatment. In certain embodiments, the method is further characterized by the feature that the incidence of any nervous system disorder due to the first therapeutic agent occurs no more frequently than one patient for every 10, 12, 14, 16, 18, 20, 22, 24, 26, 28, 30, 32, 34, 36, 38, or 40 patients subjected to the same treatment.

In certain embodiments, preventing the progression of a diabetic retinal disease is expressed as preventing the worsening of diabetic retinal disease. In certain embodiments, preventing the progression of a diabetic retinopathy is expressed as preventing the worsening of diabetic retinopathy.

Another aspect of the invention provides for the use of the first therapeutic agent described herein in the manufacture of a medicament. In certain embodiments, the medicament is for treating a disorder described herein, for example, for treating diabetic retinopathy, diabetic macular edema, and/or other diabetic retinal disorders.

Another aspect of the invention provides for the use of the first therapeutic agent described herein for treating a medical disorder, such as a medical disorder described herein, for example, for treating diabetic retinopathy, diabetic macular edema, and/or other diabetic retinal disorders.

### First Therapeutic Agent

In certain embodiments, the first therapeutic agent is a compound of Formula I or a pharmaceutically acceptable salt thereof. In certain embodiments, the first therapeutic agent is a compound of Formula I. In certain embodiments, the first therapeutic agent is a pharmaceutically acceptable salt of a compound of Formula I. In certain embodiments, the first therapeutic agent is an alkaline earth metal salt of a compound of Formula I. In certain embodiments, the first therapeutic agent is a calcium salt of a compound of Formula I.

In certain embodiments, the first therapeutic agent is crystalline.

In certain embodiments, the first therapeutic agent is in the form of a hydrate. In certain embodiments, the first therapeutic agent is a calcium salt of a compound of Formula I in the form of a hydrate.

In certain embodiments, the calcium salt of compound of Formula I exhibits an XRPD pattern comprising a peak at 4.1±0.2 degrees 2-theta, a peak at 5.3±0.2 degrees 2-theta, and a peak at 6.3±0.2 degrees 2-theta. In certain embodiments, the XRPD pattern further comprises a peak at 5.8±0.2 degrees 2-theta or a peak at 14.1±0.2 degrees 2-theta. In certain embodiments, the XRPD pattern further comprises a peak at 12.8±0.2 degrees 2-theta or a peak at 13.7±0.2 degrees 2-theta. In certain embodiments, the XRPD pattern further comprises a peak at 10.8±0.2 degrees 2-theta. In certain embodiments, the XRPD pattern further comprises a peak at 6.1±0.2 degrees 2-theta or a peak at 11.4±0.2 degrees 2-theta. In certain embodiments, the XRPD pattern further comprises a peak at 17.6±0.2 degrees 2-theta or a peak at 20.3±0.2 degrees 2-theta. In certain embodiments, the XRPD pattern further comprises a peak at 3.0±0.2 degrees 2-theta or a peak at 12.3±0.2 degrees 2-theta. In certain embodiments, the XRPD pattern further comprises a peak at 11.4±0.2 degrees 2-theta, a peak at 17.6±0.2 degrees 2-theta, and a peak at 21.9±0.2 degrees 2-theta.

In certain embodiments, the calcium salt of compound of Formula I exhibits an XRPD pattern comprising a peak at 4.1±0.2 degrees 2-theta, a peak at 5.3±0.2 degrees 2-theta, and a peak at 6.3±0.2 degrees 2-theta. In certain embodiments, the XRPD pattern further comprises a peak at 5.8±0.2 degrees 2-theta or a peak at 13.7±0.2 degrees 2-theta. In certain embodiments, the XRPD pattern further comprises a peak at 12.8±0.2 degrees 2-theta or a peak at 14.1±0.2 degrees 2-theta. In certain embodiments, the XRPD pattern further comprises a peak at 12.3±0.2 degrees 2-theta. In certain embodiments, the XRPD pattern further comprises a peak at 11.4±0.2 degrees 2-theta. In certain embodiments, the XRPD pattern further comprises a peak at 10.8±0.2 degrees 2-theta.

In certain embodiments, the calcium salt of compound of Formula I exhibits an XRPD pattern comprising a peak at 4.1±0.2 degrees 2-theta, a peak at 5.4±0.2 degrees 2-theta, and a peak at 6.4±0.2 degrees 2-theta. In certain embodiments, the XRPD pattern further comprises a peak at 5.8±0.2 degrees 2-theta or a peak at 13.8±0.2 degrees 2-theta. In certain embodiments, the XRPD pattern further comprises a peak at 12.8±0.2 degrees 2-theta or a peak at 14.2±0.2 degrees 2-theta. In certain embodiments, the XRPD pattern further comprises a peak at 12.3±0.2 degrees 2-theta. In certain embodiments, the XRPD pattern further comprises a peak at 12.0±0.2 degrees 2-theta or a peak at 11.4±0.2 degrees 2-theta. In certain embodiments, the XRPD pattern further comprises a peak at 10.8±0.2 degrees 2-theta or a peak at 20.9±0.2 degrees 2-theta. In certain embodiments, the XRPD pattern further comprises a peak at 13.1±0.2 degrees 2-theta, a peak at 17.6±0.2 degrees 2-theta, and a peak at 23.7±0.2 degrees 2-theta.

In certain embodiments, the calcium salt of compound of Formula I exhibits an XRPD pattern comprising a peak at 4.1±0.2 degrees 2-theta, a peak at 5.3±0.2 degrees 2-theta, and a peak at 6.3±0.2 degrees 2-theta, wherein the peak at 4.1±0.2 degrees 2-theta, the peak at 5.3±0.2 degrees 2-theta, and the peak at 6.3±0.2 degrees 2-theta, have a relative peak intensity (%) of greater than 30%. In certain embodiments, the XRPD pattern further comprises a peak at 5.8±0.2 degrees 2-theta or a peak at 14.1±0.2 degrees 2-theta, wherein the peak at 5.8±0.2 degrees 2-theta and the peak at 14.1±0.2 degrees 2-theta have a relative peak intensity (%) of greater than 15%. In certain embodiments, the XRPD pattern further comprises a peak at 12.8±0.2 degrees 2-theta or a peak at 13.7±0.2 degrees 2-theta, wherein the peak at 12.8±0.2 degrees 2-theta and the peak at 13.7±0.2 degrees 2-theta have a relative peak intensity (%) of greater than 10%. In certain embodiments, the XRPD pattern further comprises a peak at 10.8±0.2 degrees 2-theta, wherein the peak has a relative peak intensity (%) of greater than 10%. In certain embodiments, the XRPD pattern further comprises a peak at 6.1±0.2 degrees 2-theta or a peak at 11.4±0.2 degrees 2-theta, wherein the peak at 6.1±0.2 degrees 2-theta and the peak at 11.4±0.2 degrees 2-theta have a relative peak intensity (%) of greater than 10%. In certain embodiments, the XRPD pattern further comprises a peak at 17.6±0.2 degrees 2-theta or a peak at 20.3±0.2 degrees 2-theta, wherein the peak at 17.6±0.2 degrees 2-theta and the peak at 20.3±0.2 degrees 2-theta have a relative peak intensity (%) of greater than 8%. In certain embodiments, the XRPD pattern further comprises a peak at 3.0±0.2 degrees 2-theta or a peak at 12.3±0.2 degrees 2-theta, wherein the peak at 3.0±0.2 degrees 2-theta and the peak at 12.3±0.2 degrees 2-theta have a relative peak intensity (%) of greater than 5%. In certain embodiments, the XRPD pattern further comprises a peak at 11.4±0.2 degrees 2-theta, a peak at 17.6±0.2 degrees 2-theta, or a peak at 21.9±0.2 degrees 2-theta, wherein the peak at 11.4±0.2 degrees 2-theta, the peak at 17.6±0.2 degrees 2-theta, and the peak at 21.9±0.2 degrees 2-theta have a relative peak intensity (%) of greater than 5%.

In certain embodiments, the calcium salt of compound of Formula I exhibits an XRPD pattern comprising a peak at 4.1±0.2 degrees 2-theta, a peak at 5.4±0.2 degrees 2-theta, and a peak at 6.4±0.2 degrees 2-theta, wherein the peak at 4.1±0.2 degrees 2-theta, the peak at 5.4±0.2 degrees 2-theta, and the peak at 6.4±0.2 degrees 2-theta have a relative peak intensity (%) of greater than 30%. In certain embodiments, the XRPD pattern further comprises a peak at 5.8±0.2 degrees 2-theta or a peak at 13.8±0.2 degrees 2-theta, wherein the peak at 5.8±0.2 degrees 2-theta and the peak at 13.8±0.2 degrees 2-theta have a relative peak intensity (%) of greater than 15%. In certain embodiments, the XRPD pattern further comprises a peak at 12.8±0.2 degrees 2-theta or a peak at 14.2±0.2 degrees 2-theta, wherein the peak at 12.8±0.2 degrees 2-theta and the peak at 14.2±0.2 degrees 2-theta have a relative peak intensity (%) of greater than 10%. In certain embodiments, the XRPD pattern further comprises a peak at 12.3±0.2 degrees 2-theta, wherein the peak has a relative peak intensity (%) of greater than 10%. In certain embodiments, the XRPD pattern further comprises a peak at 12.0±0.2 degrees 2-theta or a peak at 11.4±0.2 degrees 2-theta, wherein the peak at 12.0±0.2 degrees 2-theta and the peak at 11.4±0.2 degrees 2-theta have a relative peak intensity (%) of greater than 10%. In certain embodiments, the XRPD pattern further comprises a peak at 10.8±0.2 degrees 2-theta or a peak at 20.9±0.2 degrees 2-theta, wherein the peak at 10.8±0.2 degrees 2-theta and the peak at 20.9±0.2 degrees 2-theta have a relative peak intensity (%) of greater than 6%. In certain embodiments, the XRPD pattern further comprises a peak at 13.1±0.2 degrees 2-theta, a peak at 17.6±0.2 degrees 2-theta, or a peak at 23.7±0.2 degrees 2-theta, wherein the peak at 13.1±0.2 degrees 2-theta, the peak at 17.6±0.2 degrees 2-theta, and the peak at 23.7±0.2 degrees 2-theta have a relative peak intensity (%) of greater than 5%.

In certain embodiments, the calcium salt of compound of Formula I exhibits an XRPD pattern comprising a peak at 6.3±0.2 degrees 2-theta. In certain embodiments, the calcium salt of compound of Formula I exhibits an XRPD pattern comprising a peak at 5.8±0.2 degrees 2-theta. In certain embodiments, the calcium salt of compound of Formula I exhibits an XRPD pattern comprising a peak at 13.7±0.2 degrees 2-theta. In certain embodiments, the calcium salt of compound of Formula I exhibits an XRPD pattern comprising a peak at 14.1±0.2 degrees 2-theta.

In certain embodiments, the calcium salt of compound of Formula I exhibits an XRPD pattern comprising a peak at 6.3±0.2 degrees 2-theta and a peak at 5.8±0.2 degrees 2-theta. In certain embodiments, the calcium salt of compound of Formula I exhibits an XRPD pattern comprising a peak at 6.3±0.2 degrees 2-theta and a peak at 14.1±0.2 degrees 2-theta. In certain embodiments, the calcium salt of compound of Formula I exhibits an XRPD pattern comprising a peak at 6.3±0.2 degrees 2-theta, a peak at 5.8±0.2 degrees 2-theta, and a peak at 14.1±0.2 degrees 2-theta. In certain embodiments, the calcium salt of compound of Formula I exhibits an XRPD pattern comprising a peak at 5.8±0.2 degrees 2-theta, a peak at 6.3±0.2 degrees 2-theta, and a peak at 13.7±0.2 degrees 2-theta. In certain embodiments, the calcium salt of compound of Formula I exhibits an XRPD pattern comprising a peak at 5.8±0.2 degrees 2-theta, a peak at 6.3±0.2 degrees 2-theta, a peak at 13.7±0.2 degrees 2-theta, or a peak at 14.1±0.2 degrees 2-theta. In certain embodiments, the calcium salt of compound of Formula I exhibits an XRPD pattern comprising a peak at 5.8±0.2 degrees 2-theta, a peak at 6.3±0.2 degrees 2-theta, a peak at 13.7±0.2 degrees 2-theta, and a peak at 14.1±0.2 degrees 2-theta.

In certain embodiments, the calcium salt of compound of Formula I exhibits an XRPD pattern comprising a peak at 5.8±0.2 degrees 2-theta and a peak at 6.3±0.2 degrees 2-theta. In certain embodiments, the XRPD pattern further comprises a peak at 13.7±0.2 degrees 2-theta or a peak at 14.1±0.2 degrees 2-theta.

In certain embodiments, the calcium salt of compound of Formula I exhibits an XRPD pattern comprising a peak at 5.8±0.2 degrees 2-theta and a peak at 6.3±0.2 degrees 2-theta. In certain embodiments, the XRPD pattern further comprises a peak at 13.7±0.2 degrees 2-theta or a peak at 14.1±0.2 degrees 2-theta.

In certain embodiments, the calcium salt of compound of Formula I exhibits an XRPD pattern comprising a peak at 6.4±0.2 degrees 2-theta. In certain embodiments, the calcium salt of compound of Formula I exhibits an XRPD pattern comprising a peak at 5.8±0.2 degrees 2-theta. In certain embodiments, the calcium salt of compound of Formula I exhibits an XRPD pattern comprising a peak at 13.8±0.2 degrees 2-theta. In certain embodiments, the calcium salt of compound of Formula I exhibits an XRPD pattern comprising a peak at 14.2±0.2 degrees 2-theta.

In certain embodiments, the calcium salt of compound of Formula I exhibits an XRPD pattern comprising a peak at 6.4±0.2 degrees 2-theta and a peak at 5.8±0.2 degrees 2-theta. In certain embodiments, the calcium salt of compound of Formula I exhibits an XRPD pattern comprising a peak at 6.4±0.2 degrees 2-theta and a peak at 14.2±0.2 degrees 2-theta. In certain embodiments, the calcium salt of compound of Formula I exhibits an XRPD pattern comprising a peak at 6.4±0.2 degrees 2-theta, a peak at 5.8±0.2 degrees 2-theta, and a peak at 14.2±0.2 degrees 2-theta. In certain embodiments, the calcium salt of compound of Formula I exhibits an XRPD pattern comprising a peak at 5.8±0.2 degrees 2-theta, a peak at 6.4±0.2 degrees 2-theta, and a peak at 13.8±0.2 degrees 2-theta. In certain embodiments, the calcium salt of compound of Formula I exhibits an XRPD pattern comprising a peak at 6.4±0.2 degrees 2-theta, a peak at 5.8±0.2 degrees 2-theta, a peak at 13.8±0.2 degrees 2-theta, or a peak at 14.2±0.2 degrees 2-theta. In certain embodiments, the calcium salt of compound of Formula I exhibits an XRPD pattern comprising a peak at 6.4±0.2 degrees 2-theta, a peak at 5.8±0.2 degrees 2-theta, a peak at 13.8±0.2 degrees 2-theta, and a peak at 14.2±0.2 degrees 2-theta.

In certain embodiments, the calcium salt of compound of Formula I exhibits an XRPD pattern comprising a peak at 5.8±0.2 degrees 2-theta and a peak at 6.4±0.2 degrees 2-theta. In certain embodiments, the XRPD pattern further comprises a peak at 13.8±0.2 degrees 2-theta or a peak at 14.2±0.2 degrees 2-theta.

In certain embodiments, the calcium salt of compound of Formula I exhibits an XRPD pattern comprising a peak at 5.8±0.2 degrees 2-theta and a peak at 6.4±0.2 degrees 2-theta. In certain embodiments, the XRPD pattern further comprises a peak at 13.8±0.2 degrees 2-theta or a peak at 14.2±0.2 degrees 2-theta.

In certain embodiments, the calcium salt of compound of Formula I exhibits an XRPD pattern comprising a peak at 6.3±0.2 degrees 2-theta and 1 peak, 2 peaks, 3 peaks, 4 peaks, 5 peaks, 6 peaks, 7 peaks, 8 peaks, or 9 peaks of Table 1 in Example 3. In certain embodiments, the calcium salt of compound of Formula I exhibits an XRPD pattern comprising a peak at 6.3±0.2 degrees 2-theta and 1 peak, 2 peaks, 3 peaks, 4 peaks, 5 peaks, 6 peaks, 7 peaks, 8 peaks, or 9 peaks of the following peaks: a peak at 4.1±0.2 degrees 2-theta, a peak at 5.3±0.2 degrees 2-theta, a peak at 5.8±0.2 degrees 2-theta, a peak at 6.1±0.2 degrees 2-theta, a peak at 10.8±0.2 degrees 2-theta, a peak at 11.4±0.2 degrees 2-theta, a peak at 12.8±0.2 degrees 2-theta, a peak at 13.7±0.2 degrees 2-theta, and 14.1±0.2 degrees 2-theta.

In certain embodiments, the calcium salt of compound of Formula I exhibits an XRPD pattern comprising a peak at 6.4±0.2 degrees 2-theta and 1 peak, 2 peaks, 3 peaks, 4 peaks, 5 peaks, 6 peaks, 7 peaks, 8 peaks, or 9 peaks of Table 1 in Example 3. In certain embodiments, the calcium salt of compound of Formula I exhibits an XRPD pattern comprising a peak at 6.4±0.2 degrees 2-theta and 1 peak, 2 peaks, 3 peaks, 4 peaks, 5 peaks, 6 peaks, 7 peaks, 8 peaks, or 9 peaks of the following peaks: a peak at 4.1±0.2 degrees 2-theta, a peak at 5.4±0.2 degrees 2-theta, a peak at 5.8±0.2 degrees 2-theta, a peak at 11.4±0.2 degrees 2-theta, a peak at 12.0±0.2 degrees 2-theta, a peak at 12.3±0.2 degrees 2-theta, a peak at 12.8±0.2 degrees 2-theta, a peak at 13.8±0.2 degrees 2-theta, and 14.2±0.2 degrees 2-theta.

In certain embodiments, the calcium salt of compound of Formula I exhibits an XRPD pattern comprising a Table 1 in Example 3 peak having a relative peak intensity (%) of greater than 30%. In certain embodiments, the calcium salt of compound of Formula I exhibits an XRPD pattern comprising a Table 1 in Example 3 peak having a relative peak intensity (%) of greater than 20%. In certain embodiments, the calcium salt of compound of Formula I exhibits an XRPD pattern comprising a Table 1 in Example 3 peak having a relative peak intensity (%) of greater than 15%. In certain embodiments, the calcium salt of compound of Formula I exhibits an XRPD pattern comprising a Table 1 in Example 3 peak having a relative peak intensity (%) of greater than 5%.

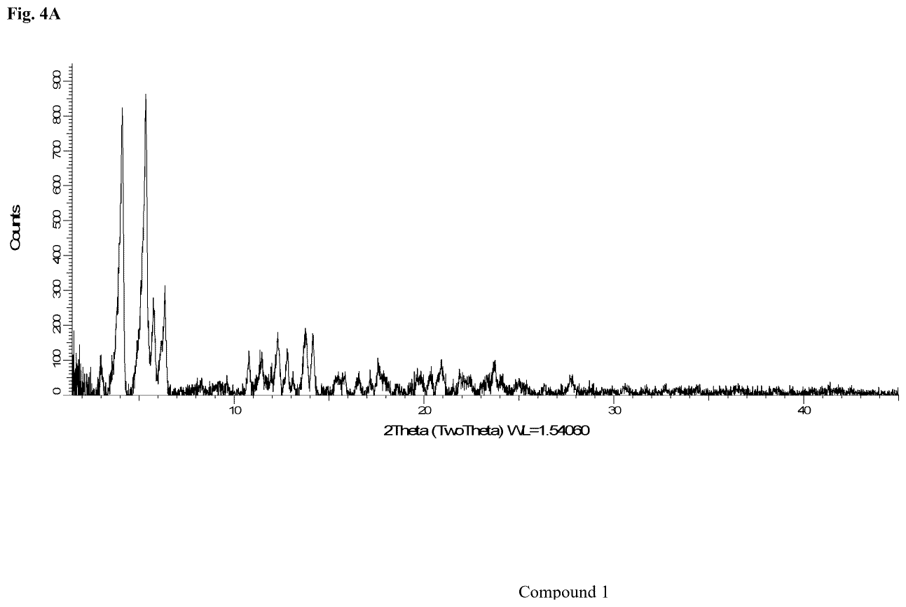

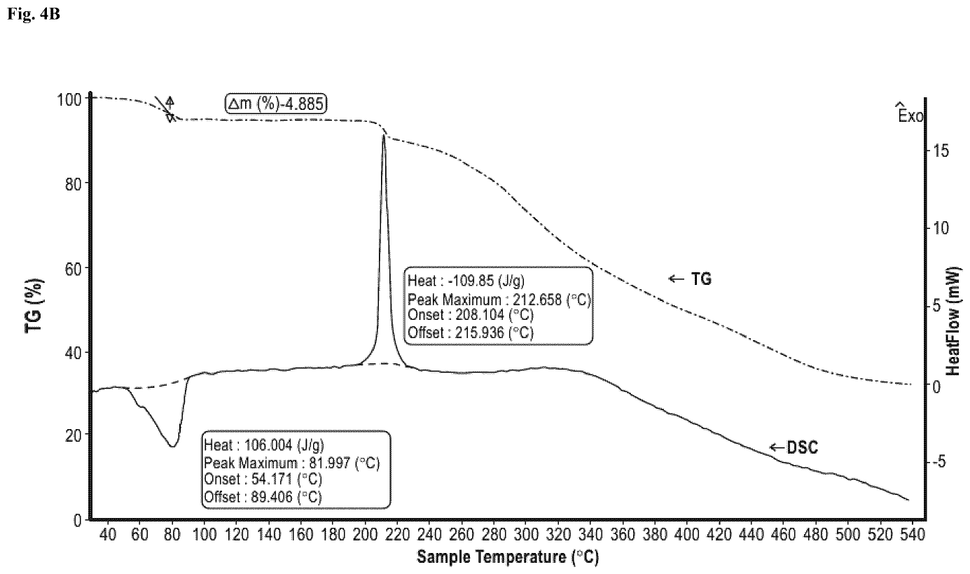

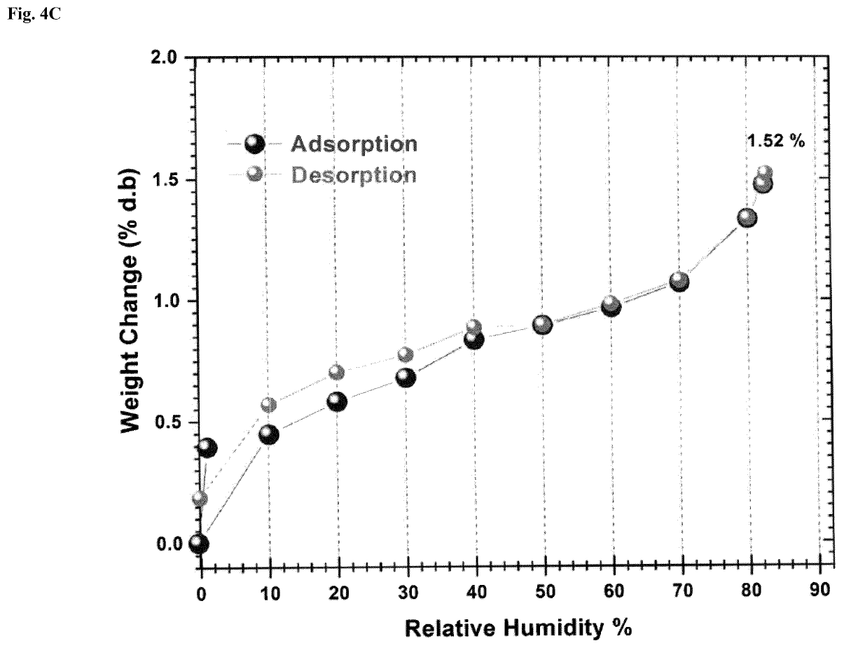

In certain embodiments, the calcium salt of compound of Formula I exhibits an XRPD pattern that is substantially the same as that depicted in FIG. 4A.

In certain embodiments, the calcium salt of compound of Formula I exhibits a DSC thermogram comprising an endothermic peak having a peak maximum of from about 80° C. to about 84° C. In certain embodiments, the calcium salt of compound of Formula I exhibits a DSC thermogram comprising an endothermic peak having a peak maximum of from about 81° C. to about 83° C. In certain embodiments, the calcium salt of compound of Formula I exhibits a DSC thermogram comprising an endothermic peak having a peak maximum of about 83° C. In certain embodiments, the calcium salt of compound of Formula I exhibits a DSC thermogram comprising an endothermic peak that onsets at about 58° C. In certain embodiments, the calcium salt of compound of Formula I exhibits a DSC thermogram comprising an endothermic peak that onsets at about 54° C.

In certain embodiments, the calcium salt of compound of Formula I exhibits a DSC thermogram comprising an exothermic peak having a peak maximum of from about 213° C. to about 216° C. In certain embodiments, the calcium salt of compound of Formula I exhibits a DSC thermogram comprising an exothermic peak having a peak maximum of from about 215° C. to about 216° C. In certain embodiments, the calcium salt of compound of Formula I exhibits a DSC thermogram comprising an exothermic peak having a peak maximum of from about 208° C. to about 216° C. In certain embodiments, the calcium salt of compound of Formula I exhibits a DSC thermogram comprising an exothermic peak having a peak maximum of from about 210° C. to about 214° C. In certain embodiments, the calcium salt of compound of Formula I exhibits a DSC thermogram comprising an exothermic peak that onsets at about 212° C.

In certain embodiments, the calcium salt of compound of Formula I exhibits a DSC thermogram that is substantially the same as that depicted in FIG. 4B. In certain embodiments, the calcium salt of compound of Formula I exhibits a thermo-gravimetric (TG) thermogram that is substantially the same as that depicted in FIG. 4B. In certain embodiments, the calcium salt of compound of Formula I exhibits a DVS spectrum that is substantially the same as that depicted in FIG. 4C.

In certain embodiments, the calcium salt of compound of Formula I is a monohydrate.

In certain embodiments, the calcium salt of compound of Formula I obtained is at least about 98% pure by weight. In certain embodiments, the calcium salt of compound of Formula I is at least about 99% pure by weight.

In certain embodiments, the calcium salt of compound of Formula I exhibits an X-ray powder diffraction (XRPD) pattern comprising a peak at 5.1±0.2 degrees 2-theta, a peak at 5.3±0.2 degrees 2-theta, and a peak at 10.1±0.2 degrees 2-theta. In certain embodiments, the XRPD pattern further comprises a peak at a peak at 8.0±0.2 degrees 2-theta or a peak at 12.8±0.2 degrees 2-theta. In certain embodiments, the XRPD pattern further comprises a peak at 13.3±0.2 degrees 2-theta, a peak at 17.9±0.2 degrees 2-theta, or a peak at 19.6±0.2 degrees 2-theta. In certain embodiments, the XRPD pattern further comprises a peak at 14.6±0.2 degrees 2-theta or a peak at 18.5±0.2 degrees 2-theta.

In certain embodiments, the calcium salt of compound of Formula I exhibits an XRPD pattern comprising a peak at 5.1±0.2 degrees 2-theta, a peak at 5.3±0.2 degrees 2-theta, and a peak at 10.1±0.2 degrees 2-theta, wherein the peak at 5.1±0.2 degrees 2-theta, the peak at 5.3±0.2 degrees 2-theta, and the peak at 10.1±0.2 degrees 2-theta have a relative peak intensity (%) of greater than 25%. In certain embodiments, the XRPD pattern further comprises a peak at 8.0±0.2 degrees 2-theta or a peak at 12.8±0.2 degrees 2-theta, wherein the peak at 8.0±0.2 degrees 2-theta and the peak at 12.8±0.2 degrees 2-theta have a relative peak intensity (%) of greater than 10%. In certain embodiments, the XRPD pattern further comprises a peak at 13.3±0.2 degrees 2-theta, a peak at 17.9±0.2 degrees 2-theta, or a peak at 19.6±0.2 degrees 2-theta, wherein the peak at 13.3±0.2 degrees 2-theta, the peak at 17.9±0.2 degrees 2-theta, and the peak at 19.6±0.2 degrees 2-theta have a relative peak intensity (%) of greater than 10%. In certain embodiments, the XRPD pattern further comprises a peak at 14.6±0.2 degrees 2-theta or a peak at 18.5±0.2 degrees 2-theta, wherein the peak at 14.6±0.2 degrees 2-theta or the peak at 18.5±0.2 degrees 2-theta have a relative peak intensity (%) of greater than 8%.

In certain embodiments, the calcium salt of compound of Formula I exhibits an XRPD pattern comprising a peak at 10.1±0.2 degrees 2-theta. In certain embodiments, the calcium salt of compound of Formula I exhibits an XRPD pattern comprising a peak at 8.0±0.2 degrees 2-theta. In certain embodiments, the calcium salt of compound of Formula I exhibits an XRPD pattern comprising a peak at 13.3±0.2 degrees 2-theta. In certain embodiments, the calcium salt of compound of Formula I exhibits an XRPD pattern comprising a peak at 14.6±0.2 degrees 2-theta.

In certain embodiments, the calcium salt of compound of Formula I exhibits an XRPD pattern comprising a peak at 8.0±0.2 degrees 2-theta and a peak at 10.1±0.2 degrees 2-theta. In certain embodiments, the calcium salt of compound of Formula I exhibits an XRPD pattern comprising a peak at 10.1±0.2 degrees 2-theta and a peak at 14.6±0.2 degrees 2-theta. In certain embodiments, the calcium salt of compound of Formula I exhibits an XRPD pattern comprising a peak at 8.0±0.2 degrees 2-theta, a peak at 10.1±0.2 degrees 2-theta, and a peak at 13.3±0.2 degrees 2-theta. In certain embodiments, the calcium salt of compound of Formula I exhibits an XRPD pattern comprising a peak at 8.0±0.2 degrees 2-theta, a peak at 10.1±0.2 degrees 2-theta, and a peak at 14.6±0.2 degrees 2-theta. In certain embodiments, the calcium salt of compound of Formula I exhibits an XRPD pattern comprising a peak at 8.0±0.2 degrees 2-theta, a peak at 10.1±0.2 degrees 2-theta, a peak at 13.3±0.2 degrees 2-theta, or a peak at 14.6±0.2 degrees 2-theta. In certain embodiments, the calcium salt of compound of Formula I exhibits an XRPD pattern comprising a peak at 8.0±0.2 degrees 2-theta, a peak at 10.1±0.2 degrees 2-theta, a peak at 13.3±0.2 degrees 2-theta, and a peak at 14.6±0.2 degrees 2-theta.

In certain embodiments, the calcium salt of compound of Formula I exhibits an XRPD pattern comprising a peak at 10.1±0.2 degrees 2-theta and 1 peak, 2 peaks, 3 peaks, 4 peaks, 5 peaks, 6 peaks, 7 peaks, 8 peaks, or 9 peaks of Table 3 in Example 4. In certain embodiments, the calcium salt of compound of Formula I exhibits an XRPD pattern comprising a peak at 10.1±0.2 degrees 2-theta and 1 peak, 2 peaks, 3 peaks, 4 peaks, 5 peaks, 6 peaks, 7 peaks, 8 peaks, or 9 peaks of the following peaks: a peak at 5.1±0.2 degrees 2-theta, a peak at 5.3±0.2 degrees 2-theta, a peak at 7.9±0.2 degrees 2-theta, a peak at 12.8±0.2 degrees 2-theta, a peak at 13.3±0.2 degrees 2-theta, a peak at 14.6±0.2 degrees 2-theta, a peak at 17.9±0.2 degrees 2-theta, a peak at 18.5±0.2 degrees 2-theta, and a peak at 19.6±0.2 degrees 2-theta.

In certain embodiments, the calcium salt of compound of Formula I exhibits an XRPD pattern comprising a peak from Table 3 in Example 4 having a relative peak intensity (%) of greater than 25%. In certain embodiments, the calcium salt of compound of Formula I exhibits an XRPD pattern comprising a peak from Table 3 in Example 4 having a relative peak intensity (%) of greater than 15%. In certain embodiments, the calcium salt of compound of Formula I exhibits an XRPD pattern comprising a peak from Table 3 in Example 4 having a relative peak intensity (%) of greater than 12%.

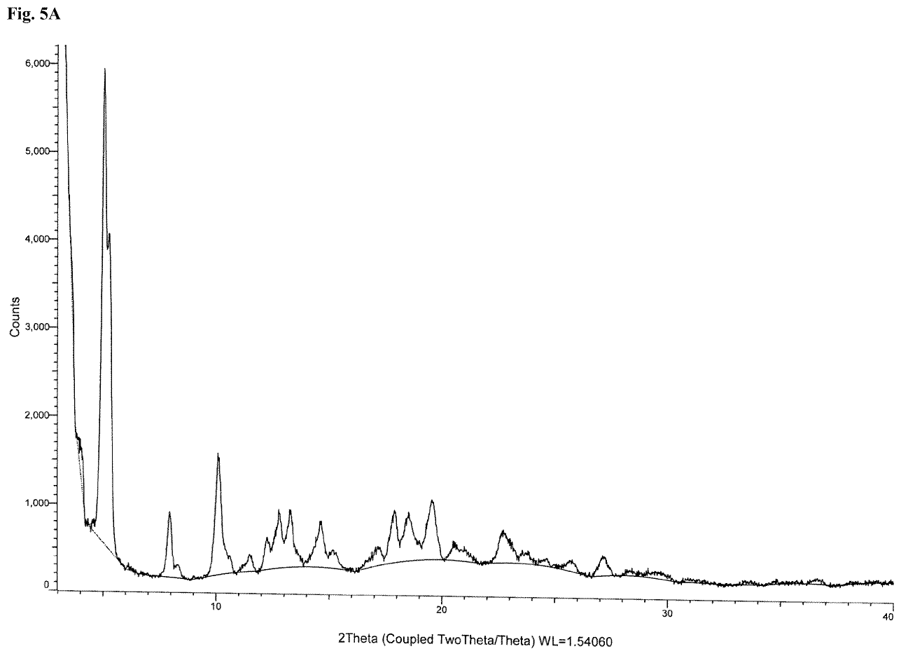

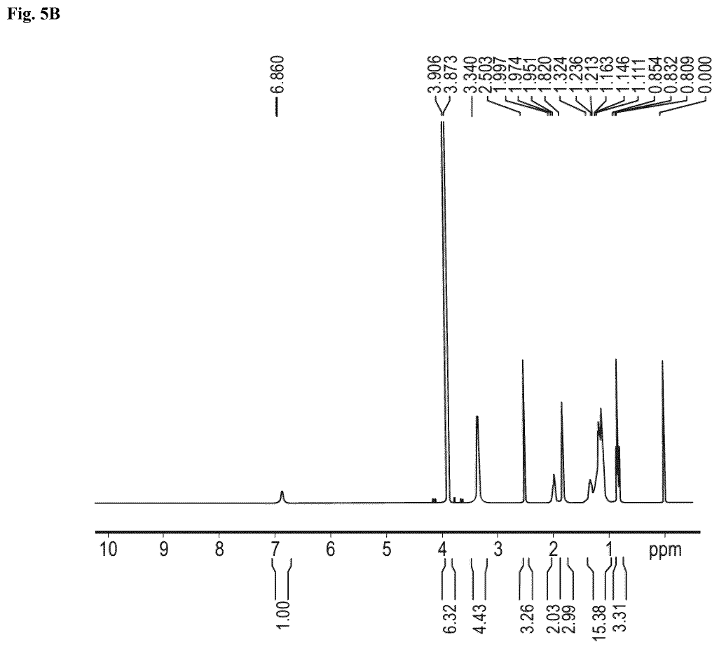

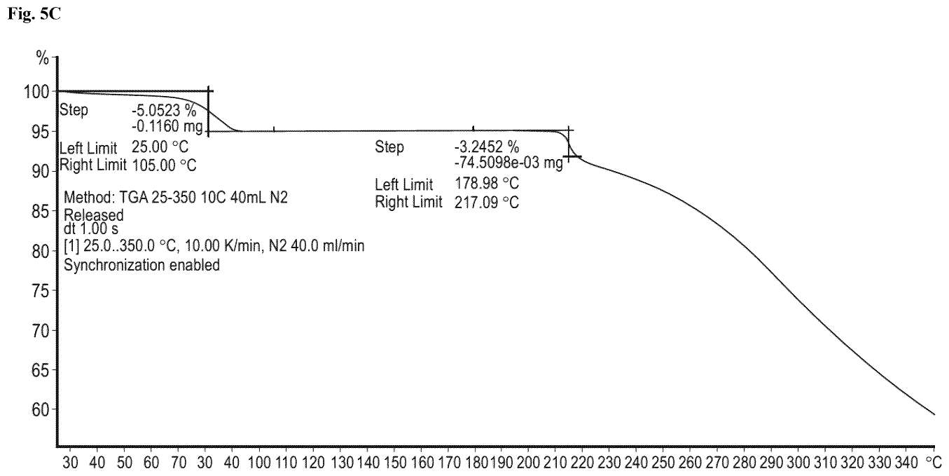

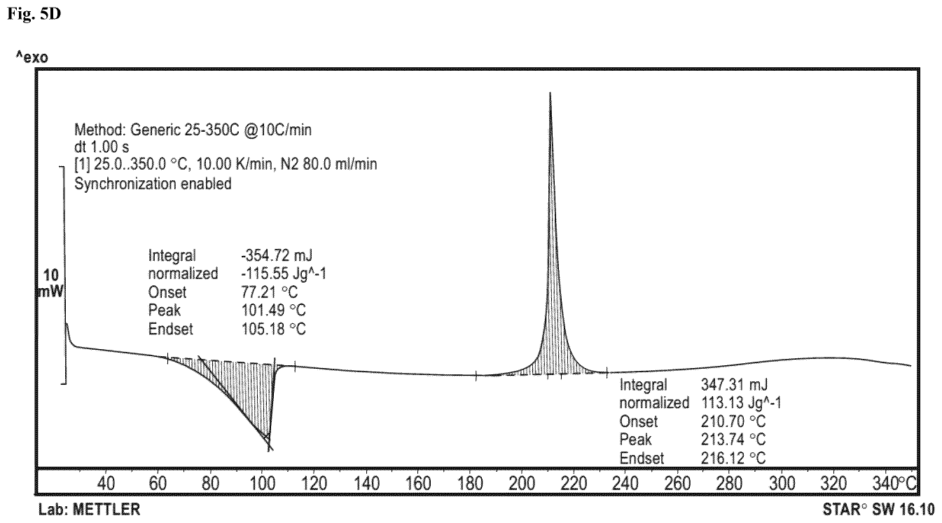

In certain embodiments, the calcium salt of compound of Formula I exhibits an XRPD pattern that is substantially the same as that depicted in FIG. 5A.

In certain embodiments, the calcium salt of compound of Formula I exhibits a differential scanning calorimetry (DSC) thermogram comprising an endothermic peak having a peak maximum of from about 80° C. to about 105° C. In certain embodiments, the calcium salt of compound of Formula I exhibits a DSC thermogram comprising an endothermic peak having a peak maximum of from about 90° C. to about 105° C. In certain embodiments, the calcium salt of compound of Formula I exhibits a DSC thermogram comprising an endothermic peak having a peak maximum of from about 95° C. to about 105° C. In certain embodiments, the calcium salt of compound of Formula I exhibits a DSC thermogram comprising an endothermic peak having a peak maximum of from about 98° C. to about 103° C. In certain embodiments, the calcium salt of compound of Formula I exhibits a DSC thermogram comprising an endothermic peak having a peak maximum of about 101° C. In certain embodiments, the calcium salt of compound of Formula I exhibits a DSC thermogram comprising an endothermic peak that onsets at about 77° C.

In certain embodiments, the calcium salt of compound of Formula I exhibits a DSC thermogram comprising an exothermic peak having a peak maximum of from about 210° C. to about 216° C. In certain embodiments, the calcium salt of compound of Formula I exhibits a DSC thermogram comprising an exothermic peak having a peak maximum of from about 212° C. to about 215° C. In certain embodiments, the calcium salt of compound of Formula I exhibits a DSC thermogram comprising an exothermic peak having a peak maximum of from about 213° C. to about 215° C. In certain embodiments, the calcium salt of compound of Formula I exhibits a DSC thermogram comprising an exothermic peak having a peak maximum at about 214° C. In certain embodiments, the calcium salt of compound of Formula I exhibits a DSC thermogram comprising an exothermic peak that onsets at about 211° C.

In certain embodiments, the calcium salt of compound of Formula I exhibits a DSC thermogram that is substantially the same as that depicted in FIG. 5D. In certain embodiments, the calcium salt of compound of Formula I exhibits a thermo-gravimetric (TG) thermogram that is substantially the same as that depicted in FIG. 5C.

In certain embodiments, the calcium salt of compound of Formula I is a monohydrate.

In certain embodiments, the calcium salt of compound of Formula I has a solubility of about 500 μg/mL to about 600 μg/mL at pH 5 and 37° C., after 4 hours of being added to a pH 5 and 37° C. solution of Fed State Simulated Intestinal Fluid (FeSSIF) when determined by high-performance liquid chromatography (HPLC) at 236 nm or at 266 nm. In certain embodiments, the calcium salt of compound of Formula I has a solubility of about 600 μg/mL to about 700 μg/mL at pH 5 and 37° C., after 24 hours of being added to a pH 5 and 37° C. solution of FeSSIF when determined by HPLC at 236 nm or at 266 nm.

In certain embodiments, the calcium salt of compound of Formula I has a solubility of about 250 μg/mL to about 350 μg/mL at pH 5 and 25° C., after 4 hours of being added to a pH 5 and 25° C. solution of FeSSIF when determined by HPLC at 236 nm or at 266 nm. In certain embodiments, the calcium salt of compound of Formula I has a solubility of about 250 μg/mL to about 350 μg/mL at pH 5 and 25° C., after 24 hours of being added to a pH 5 and 25° C. solution of FeSSIF when determined by HPLC at 236 nm or at 266 nm.

In certain embodiments, the calcium salt of compound of Formula I has a solubility of about 350 μg/mL to about 450 μg/mL at pH 6.5 and 37° C., after 4 hours of being added to a pH 6.5 and 37° C. solution of Fasted State Simulated Intestinal Fluid (FaSSIF) when determined by HPLC at 236 nm or at 266 nm. In certain embodiments, the calcium salt of compound of Formula I has a solubility of about 300 μg/mL to about 400 μg/mL at pH 6.5 and 37° C., after 24 hours of being added to a pH 6.5 and 37° C. solution of FaSSIF when determined by HPLC at 236 nm or at 266 nm.

In certain embodiments, the calcium salt of compound of Formula I has a solubility of about 300 μg/mL to about 400 μg/mL at pH 6.5 and 25° C., after 4 hours of being added to a pH 6.5 and 25° C. solution of FaSSIF when determined by HPLC at 236 nm or at 266 nm. In certain embodiments, the calcium salt of compound of Formula I has a solubility of about 200 μg/mL to about 300 μg/mL at pH 6.5 and 25° C., after 24 hours of being added to a pH 6.5 and 25° C. solution of FaSSIF when determined by HPLC at 236 nm or at 266 nm.

In certain embodiments, the calcium salt of compound of Formula I is at least about 98% pure by weight, and the calcium salt comprises no more than about 2% of an impurity by weight of the calcium salt. In certain embodiments, the calcium salt of compound of Formula I is about 95.0% to 100% pure by weight, and the calcium salt comprises 0% to about 5% of an impurity by weight of the calcium salt. In certain embodiments, the calcium salt of compound of Formula I is about 98% to 100% pure by weight, and the calcium salt comprises 0% to about 2% of an impurity by weight of the calcium salt. In certain embodiments, the calcium salt of compound of Formula I is about 98%, about 98.5%, about 99%, about 99.5%, or 100% pure by weight, and the calcium salt comprises about 2%, about 1.5%, about 1%, about 0.5%, or 0%, respectively, of an impurity by weight of the calcium salt. In certain embodiments, the calcium salt of compound of Formula I is about 99.5%, about 99.9%, or about 99.95% pure by weight, and the calcium salt comprises about 0.5%, about 0.1%, or about 0.05%, respectively, of an impurity by weight of the calcium salt. In certain embodiments, the purity or the impurity are determined by high-performance liquid chromatography (HPLC). In certain embodiments, the purity or impurity is determined by HPLC at 236 nm. In certain embodiments, the purity or impurity is determined by HPLC at 266 nm. In certain embodiments, the purity or impurity is determined by titration.

In certain embodiments, the calcium salt of compound of Formula I is at least about 98% pure by weight after being exposed to 40° C./75% RH for 1 week. In certain embodiments, the calcium salt of compound of Formula I is at least about 99% pure by weight after being exposed to 40° C./75% RH for 1 week.

In certain embodiments, the calcium salt of compound of Formula I is amorphous.

In certain embodiments, the calcium salt of compound of Formula I is predominantly amorphous, wherein the predominantly amorphous calcium salt exhibits an XRPD pattern comprising a peak at 4.5±0.2 degrees 2-theta and a peak at 6.0±0.2 degrees 2-theta.

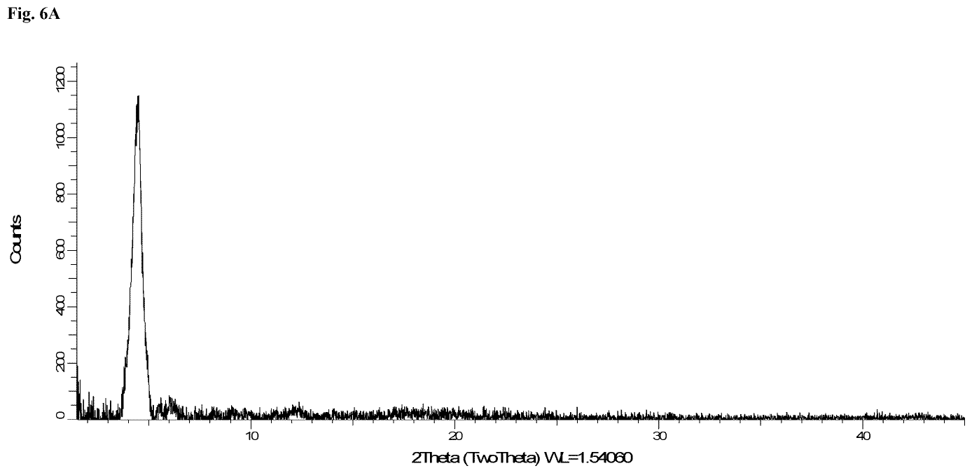

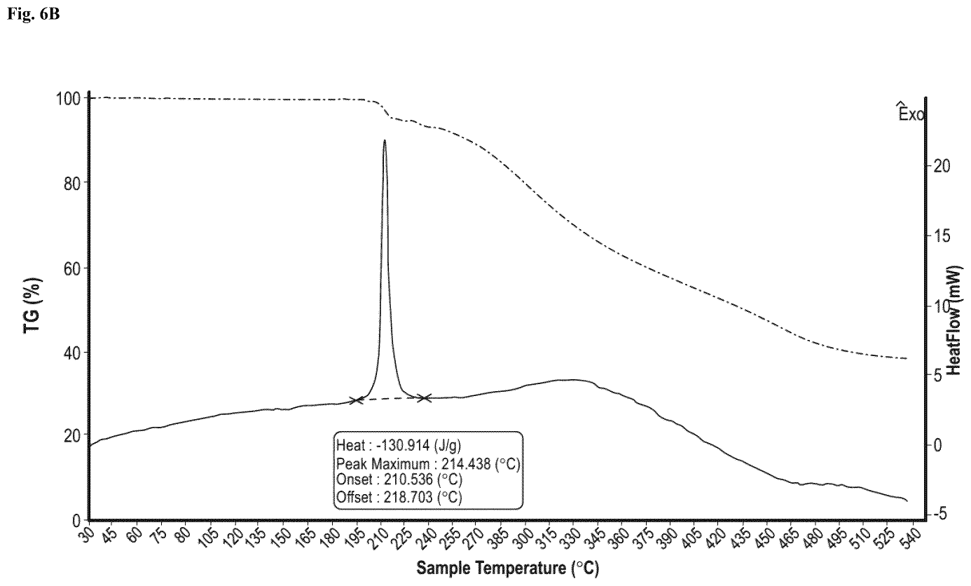

In certain embodiments, the predominantly amorphous calcium salt of compound of Formula I exhibits an XRPD pattern that is predominantly the same as that depicted in FIG. 6A.

In certain embodiments, the predominantly amorphous calcium salt of compound of Formula I is an anhydrate.

In certain embodiments, the predominantly amorphous calcium salt of compound of Formula I is at least 50% amorphous by weight. In certain embodiments, the predominantly amorphous calcium salt of compound of Formula I is at least 60% amorphous by weight. In certain embodiments, the predominantly amorphous calcium salt of compound of Formula I is at least 70% amorphous by weight. In certain embodiments, the predominantly amorphous calcium salt of compound of Formula I is at least 80% amorphous by weight. In certain embodiments, the predominantly amorphous calcium salt of compound of Formula I is at least 90% amorphous by weight. In certain embodiments, the predominantly amorphous calcium salt of compound of Formula I is at least 95% amorphous by weight. In certain embodiments, the predominantly amorphous calcium salt of compound of Formula I is predominantly amorphous.

In certain embodiments, the predominantly amorphous calcium salt of compound of Formula I is a calcium salt anhydrate.

### II. Combination Therapy

Another aspect of the invention provides for combination therapy. The First, Second, Third, Fourth, Fifth, Sixth, and Seventh Therapeutic Methods described hereinabove may optionally further comprise administering one or more second therapeutic agents to the patient. For example, in certain embodiments, the method further comprises administering to the patient a second therapeutic agent for treating diabetic retinal disease.

In certain embodiments, the second therapeutic agent that is an anti-inflammatory agent, anti-angiogenic agent, tyrosine kinase inhibitor, angiopoietin-2 inhibitor, and/or vascular endothelial growth factor inhibitor. In certain embodiments, the second therapeutic agent is a vascular endothelial growth factor inhibitor. In certain embodiments, the vascular endothelial growth factor inhibitor is sorafenib, sunitinib, pazopanib, bevacizumab, ranibizumab, aflibercept, nilotinib, or dasatinib. In certain embodiments, the vascular endothelial growth factor inhibitor is a bispecific antibody. In certain embodiments, the anti-inflammatory agent is a corticosteroid. In certain embodiments, the second therapeutic agent is a VEGF inhibitor, mTor inhibitor, VEGFR2 phosphorylation agent, tyrosine kinase inhibitor, IGF-1R inhibitor, nicotinic acetylcholine receptor antagonist, selective inhibitor of glycation, corticosteroid, NSAID, flavonoid, TNF alpha inhibitor, PKC inhibitor, aldose reductase, PARP inhibitor, reactive oxygen species inhibitor, AT-I Receptor modulator, AT-II receptor modular, rho associated protein kinase inhibitor, protease inhibitor, nitric oxide synthase inhibitor, AGE inhibitor, or PPAR-gamma up-regulator.

In certain embodiments, the second therapeutic agent is an immunooncology therapy, a Car-t therapy, a Crispr therapy, a BTK modulator, a bcl-2 modulator, a stat-3 modulator, a KRAS modulator, a PD1 modulator, and/or a DNA repair agent. In certain embodiments, the second therapeutic agent is a bone marrow transplant or related transplant. In certain embodiments, the modulator is an inhibitor.

In certain embodiments, the method further comprises administering to the patient a second therapeutic agent that is an anti-inflammatory agent, anti-angiogenic agent, tyrosine kinase inhibitor, angiopoietin-2 inhibitor, rho kinase inhibitor, plasma kallikrein inhibitor, and/or vascular endothelial growth factor inhibitor. In certain embodiments, the method further comprises administering to the patient a second therapeutic agent that is a vascular endothelial growth factor inhibitor. In certain embodiments, the vascular endothelial growth factor inhibitor is sorafenib, sunitinib, pazopanib, bevacizumab, ranibizumab, aflibercept, nilotinib, or dasatinib. In certain embodiments, the vascular endothelial growth factor inhibitor is a bispecific antibody. In certain embodiments, the anti-inflammatory agent is a corticosteroid.

In certain embodiments, the method further comprises administering to the patient a second therapeutic agent that is a retinal gene therapy or retinal cell therapy.

In certain embodiments, the first therapeutic agent is the only therapeutic agent for treating diabetic retinal disease that is administered to the human patient.

In certain embodiments, such as when treating an inflammatory skin disease, the second therapeutic agent is an immunosuppressant, anti-inflammatory agent, light therapy (e.g., sunlight, UVA, UVB, Psoralen UVA, or Excimer laser), a retinoid, a corticosteroid, a Vitamin D analogue, a calcineurin inhibitor, salicylic acid, anthralin, coal tar, or Goeckerman therapy (e.g., light and coal tar).

The second therapeutic agent and optionally additional therapeutic agents may be administered separately from a compound or composition of the invention, as part of a multiple dosage regimen. Alternatively, the second therapeutic agent and optionally additional therapeutic agents may be part of a single dosage form, mixed together with a compound of this invention in a single composition. If administered as a multiple dosage regime, the second therapeutic agent and optionally additional therapeutic agents and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In certain embodiments, the second therapeutic agent and optionally additional therapeutic agents and a compound or composition of the invention are administered as a multiple dosage regimen more than 24 hours apart.

In certain embodiments, administration of the first therapeutic agent in combination with the second therapeutic agent permits administering the second therapeutic agent at a lower dose and/or less frequently. In certain embodiments, administration of the first therapeutic agent in combination with the second therapeutic agent permits administering the second therapeutic agent at a lower dose and/or less frequently while still achieving a therapeutic result for the patient that is as good or better than the result achieved with monotherapy using the second therapeutic agent.

### III. Pharmaceutical Compositions

As indicated above, the invention provides pharmaceutical compositions, which comprise a therapeutically effective amount of one or more of the compounds described above, formulated together with one or more pharmaceutically acceptable carriers (additives) and/or diluents. The pharmaceutical compositions may be specially formulated for administration in solid or liquid form, including those adapted for oral administration, for example, drenches (aqueous or non-aqueous solutions or suspensions), tablets, e.g., those targeted for buccal, sublingual, and systemic absorption, boluses, powders, granules, pastes for application to the tongue.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions.

In certain embodiments, the pharmaceutical composition may be in the form of a cream, colloidal, suspension, spray, gel, lotion, ointment, foam, or solution. In certain embodiments, the pharmaceutical composition may be in the form of a solution for injection. In certain embodiments, the pharmaceutical composition may be in the form of a solution for sub-cutaneous injection.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

In general, a suitable daily dose of a compound of the invention will be that amount of the compound which is the lowest dose effective to produce a therapeutic effect. Such an effective dose will generally depend upon the factors described above. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms.

The invention further provides a unit dosage form (such as a tablet or capsule) comprising a compound described herein in a therapeutically effective amount for the treatment of a medical disorder described herein.

### IV. Medical Kits

Another aspect of the invention provides a medical kit comprising, for example, (i) a therapeutic agent described herein, and (ii) instructions for treating diabetic retinopathy, diabetic macular edema, and/or other diabetic retinal disorders according to methods described herein.

### V. Processing of Clinical Data

Data collected during a clinical study may be processed as described below. Such data processing can be used to evaluate performance of compounds and therapeutic methods described herein.

The following analysis populations can be defined:

### Modified Intention-to-Treat (mITT)

The mITT Population includes all randomized subjects who received at least 1 dose of study treatment and at least 1 post-treatment DRSS evaluation. The mITT Population may be used for primary endpoint analysis and to analyze other efficacy endpoints, with subjects included in their randomized treatment regardless of the treatment they actually received.

### Per Protocol Population (PP)

The PP Population includes all subjects in the mITT Population who have missed less than 20% of expected doses and do not have any major protocol deviations considered to have significant impact on treatment outcome. The PP population may be used to analyze efficacy endpoints, with subjects included in their randomized treatment regardless of the treatment they actually received.

### Completer Population

The Completer Population includes all subjects in the PP Population who have at least 1 DRSS measurement at Week 48 and no rescue for PDR in the study eye. The Completer population may be to analyze efficacy endpoints, with subjects included in their randomized treatment regardless of the treatment they actually received.

### All Randomized Population (ARP)

The ARP includes all randomized subjects. This population is also known as the Intent-to-Treat (ITT) population.

### Safety Population (SP)

The SP includes all randomized subjects who have received at least one dose of study treatment. The SP may be used to summarize safety variables, using the actual treatment a subject received.

### Pharmacokinetic Population (PK)

The PK Population includes all randomized subjects who received at least one dose of study treatment and have at least one measurable study drug concentration in plasma. The PK population may be used to summarize PK variables, using the actual treatment a subject received.

### Planned Covariates

Planned covariates include baseline values for the given assessment.

### Planned Subgroups

Subgroup analyses by disease severity in the study eye (DRSS level 47 or 53) may be completed for efficacy endpoints. Other possible subgroups include age, sex, and race.

### Management of Analysis Data

Data from unscheduled visits will generally not be included in the analysis of efficacy or safety. However, if unscheduled efficacy assessments are performed in conjunction with initiation of rescue treatment for PDR, the data may be used for efficacy analyses. All data from unscheduled visits may be listed.

One primary efficacy endpoint that may be used is the percentage of subjects with a ≥3-step worsening from baseline on the DRSS Person Scale compared to baseline at Week 48.

### Partial or Missing Dates

Missing portions of dates for AEs or concomitant medications is not formally imputed. Instead, an AE may be classified as treatment-emergent or a medication as concomitant using the most conservative date that can be derived from the non-missing portion of the date.

### Missing Baseline Data

Every effort is made to ensure that accurate baseline information on the subjects is collected. If a subject is missing baseline information, the subject may be included in the SP for assessment of safety and excluded from the primary analyses. Each case of missing baseline data may be evaluated for potential inclusion in the exploratory endpoints. All baseline data are observed, without imputation.

### Imputation Methods

Imputation for efficacy data may be performed for the primary efficacy endpoint using the mITT and ARP. Multiple imputation may be employed to analyze incomplete data sets under the assumption that the mechanism responsible for the missing data is at worst characterized as missing at random (MAR).

Multiple imputation is a simulation-based approach where missing values are replaced by multiple Bayesian draws from the conditional distribution of missing data given the observed data and covariates, creating multiple completed data sets. These completed datasets can then be analyzed using standard analysis methods.

Missing values for DRSS Person Scale may be imputed simultaneously based on an underlying joint normal distribution using a Markov Chain Monte Carlo (MCMC) method.

The imputations may be done separately for each treatment group and will include the DRSS Person Scale at Baseline, Week 12, Week 24, and Week 48 in the imputation model. The number of imputations may be set to 20. The observed outcome (yes/no) at Week 48 for each imputed dataset may be evaluated using logistic regression. The estimates and standard errors (SEs) of the odds ratios based on the 20 imputed datasets are then combined by applying Rubin's rules for multiple imputed datasets. T-tests are also provided. SAS Proc MI, Proc LOGISTIC, and Proc MIANALYZE may be utilized for these analyses. The odds ratios with the corresponding 95% CI will also be presented.

A control-based Pattern-Mixture Model (PMM) may be used as a sensitivity analysis to explore the possibility of data missing not at random (MNAR) for subjects who discontinued study medication or who had a rescue treatment for PDR. For subjects who discontinued the study medication without any further follow-up data, their missing values after discontinuation may be imputed under the assumption that their outcome would be the similar to those in the placebo group with similar background characteristics. Baseline, Week 12, Week 24, and Week 48 DRSS Person Scale values may be included in a multiple imputation procedure (SAS PROC MI).

If required, intermittent missing data (Week 12 and Week 24) may be imputed using MCMC methods, assuming MAR, within each treatment group. SAS PROC MI may be utilized for this step using the MCMC impute=monotone option.

The remaining missing values with a monotone missing data pattern will then be imputed. The missing values in the Active Compound group will not be constructed from the observed data in the Active Compound group, but rather from the observed data in the placebo group. This model will also be used to impute missing efficacy measurements in the placebo group. The MNAR statement in SAS PROC MI may be used to impute missing all missing values.

Datasets may be analyzed using the logistic regression model described above, and treatment effects (difference in LS means between treatments) from these analyses will then be combined using Rubin's Method via SAS PROC MIANALYZE procedure.

Assuming the primary efficacy analysis shows a significant difference between treatment groups, a tipping point analysis will also be used as a sensitivity analysis. In this analysis, the percentage of subjects with missing data may be assumed to have increasingly negative outcomes, and the Proc LOGISTIC and Proc MIANALYZE statements described above may be utilized to determine if a significant treatment difference still exists. The tipping point is the point at which the percentage of missing data with negative outcomes shows a non-significant difference between treatment groups.

### Efficacy Analysis

Efficacy can be assessed using the mITT, PP, and Completer populations with subjects included in the treatment group in which they were randomized.

### Estimand for the Primary Efficacy Endpoint

The primary efficacy estimand, corresponding to the primary efficacy endpoint, is defined by the following components:
1. The population is restricted to the mITT population.
2. The variable for the primary endpoint is ‘Percent of subjects with a ≥3-step worsening from baseline on the DRSS Person Scale at Week 48’.
3. The intercurrent event of treatment discontinuation or initiation of rescue treatment for DR progression in either eye. Any observations taken after initiation of rescue treatment for PDR may be excluded from analysis.
4. The population-level summary for the primary efficacy endpoint is the difference in percent of subjects with a ≥3-step worsening from baseline on the DRSS Person Scale at Week 48, analyzed using a logistic regression model with fixed effects for treatment, baseline level of disease severity in the worse eye (DRSS level 47 or 53), and baseline DRSS Person Scale as a continuous covariate.
5. Treatment: There may be 1 active treatment group (APX3330) and 1 control group (placebo).
   This comparison may be analyzed first using a hierarchical testing method.

### Estimands for Secondary Efficacy Endpoints

Secondary efficacy estimands for categorical data, corresponding to the categorical secondary efficacy endpoints, are defined similarly to the primary efficacy estimand. Here are the components corresponding the secondary endpoint ‘Percent of subjects developing PDR or vitreous hemorrhage or requiring vitrectomy or PRP in either eye during the treatment period’:
1. The population is restricted to the mITT population.
2. The variable for the secondary endpoint is ‘Percent of subjects developing PDR or vitreous hemorrhage or requiring vitrectomy or PRP in either eye during the treatment period’.
3. The intercurrent event of treatment discontinuation. Any observations taken after treatment discontinuation may be excluded from analysis.
4. The population-level summary for the initial primary efficacy endpoint is the difference in percent of subjects developing PDR or vitreous hemorrhage or requiring vitrectomy or PRP in either eye during the treatment period, analyzed using a logistic regression model with fixed effects for treatment and baseline level of disease severity in the worse eye (DRSS level 47 or 53).
5. Treatment: There may be 1 active treatment group (APX3330) and 1 control group (placebo).

Estimands for other categorical secondary endpoints may be similar but components such as intercurrent events and the population-level summary may be customized for the endpoint.

Secondary efficacy estimands for continuous data, corresponding to the continuous secondary efficacy endpoints, may use different analysis methods in the population-level summary component. Here are the components corresponding the secondary endpoint ‘Mean change from baseline on the DRSS Person Scale at Week 48’:
1. The population is restricted to the mITT population.
2. The variable for the secondary endpoint is ‘Mean change from baseline on the DRSS Person Scale at Week 48’.
3. The intercurrent event of treatment discontinuation or initiation of rescue treatment for DR progression in either eye. Any observations taken after initiation of rescue treatment for PDR may be excluded from analysis.
4. The population-level summary for the initial primary efficacy endpoint is the difference in the change from baseline on the DRSS Person Scale at Week 48, analyzed using an analysis of covariance (ANCOVA) with fixed effects for treatment, baseline level of disease severity in the worse eye (DRSS level 47 or 53), and baseline DRSS Person Scale as a continuous covariate.
5. Treatment: There may be 1 active treatment group (APX3330) and 1 control group (placebo).

Estimands for other continuous secondary endpoints may be similar, but some components, such as the analysis model in the population-level summary, may be customized for the endpoint as required.

### Primary Efficacy

The primary efficacy endpoint is the difference between treatment groups in percent of subjects with a ≥3-step worsening from baseline on the DRSS Person Scale at Week 48.

For the analysis of the primary efficacy endpoint, appropriate imputation techniques may be performed for missing observations or for eyes requiring rescue treatment for PDR if applicable. If the analysis using the mITT population shows a positive effect for Active Compound at the 0.05 level of significance, the primary endpoint may be considered met. Sensitivity analyses using a control-based PMM, is also completed.

The primary efficacy endpoint may be analyzed using a logistic regression model with fixed effects for treatment and baseline level of disease severity in the worse eye (DRSS level 47 or 53), and baseline DRSS Person Scale as a covariate. The percent of subjects in each treatment group meeting the criteria, the odds ratio (OR) with 95% confidence interval (CI), and p-value may be provided.

### Secondary and Exploratory Efficacy

Each of the continuous secondary efficacy endpoints may be analyzed using ANCOVA with change from baseline as the dependent variable, treatment and baseline level of disease severity in the worse eye (DRSS level 47 or 53) as factors, and the respective baseline value included as the covariate. The output from each ANCOVA may include the least-squares mean (LSM) and standard error for both treatment groups, along with the placebo-corrected LSM, its 95% CI, and associated p-value.

For each of the secondary endpoints related to percent of subjects achieving certain criteria, the analysis may be performed using a logistic regression model with treatment and baseline level of disease severity in the worse eye (DRSS level 47 or 53) as factors, and the respective baseline as a covariate. For each analysis, the percentage of subjects in each treatment group meeting the criteria, the OR with 95% CI and p-value may be provided.

If there are sufficient missing data, appropriate MI methods may be applied, and analyses using a control-based PMM may also be applied as sensitivity analyses. The models may be similar to what is described for the primary efficacy endpoint, adjusting for the type of endpoint (categorical or continuous) and the visit collection schedule for the endpoint.

The analysis of time to event efficacy endpoints (e.g., Time to development of CI-DME, Time to development of PDR or PDR-related AEs, Time to first rescue treatment for PDR in either eye) may be performed using a Cox proportional hazards regression model with treatment and baseline level of disease severity in the worse eye (DRSS level 47 or 53) as factors. Events occurring prior to study drug administration may be excluded from the analysis but may be listed. Subjects who do not have the event may have their time censored at the date of last dose of study treatment.

The output from the model may include the hazard ratio comparing treatment groups, its 95% CI and associated p-value. Survival plots, generated from the Cox model described above, may also be generated.

Relationships between Active Compound concentrations and ERG DR scores and the change from baseline in DRSS Person Scale may be explored using scatterplots or other displays. Scatterplots may include a simple linear regression line and a correlation value.

A subset of treatment comparisons for secondary endpoints may be analyzed using a hierarchical testing method. Details may be provided prior to database lock.

### Safety Analyses

All safety analyses may be completed using the actual treatment a subject received. Observed case data may be used; no imputation may be performed for missing safety data except for limited situations. All safety data may be presented in by-subject listings. Unscheduled assessments are not summarized but may be included in the listings.

### Adverse Events

Treatment-emergent adverse events (TEAEs) are defined as any AE that begins or worsens after initiation of the investigational product and through the subject's last study visit (study completion/follow-up phone call, or early termination). Only TEAEs may be summarized in the tables and listings.

The number and percent of subjects with any TEAEs may be summarized by SOC and PT by treatment group. At each level of tabulation (e.g., at the PT level), subjects may be counted only once if they had more than one such event reported during the AE collection period. This summary may be repeated for ocular TEAEs occurring in the study eye, and then in the fellow eye; any OU TEAEs may be counted in both the study eye and the fellow eye.

The following summary tables may be presented for TEAE data, by SOC and PT:
TEAEs
Serious TEAEs
TEAEs leading to withdrawal from the study
TEAEs leading to study medication discontinuation
Treatment-related severe TEAEs
Treatment-related serious TEAEs
Treatment-related TEAEs leading to withdrawal from the study
Treatment-related TEAEs leading to study medication discontinuation
TEAEs by maximum severity (mild, moderate, severe) Treatment-related TEAEs by maximum severity
TEAEs by greatest relationship (not related, unlikely unrelated, possibly related, probably related, definitely related, or unknown).

Treatment-related TEAEs are defined as TEAEs with relationship to study medication possibly related, probably related, or definitely related.

### Deaths

Fatal TEAEs, regardless of causality, may be presented in a by-subject listing.

### Serious Adverse Events

Treatment-related treatment-emergent SAEs may be presented in a by-subject listing.

### Adverse Events Leading to Withdrawal from the Study

Treatment-emergent AEs leading to withdrawal may be presented in a by-subject listing.

### Adverse Events Leading to Discontinuation of Study Medication

Treatment-emergent AEs leading to discontinuation of study medication may be presented in a by-subject listing.

### Vital Signs

Descriptive statistics of observed values may be presented for vital sign data at each visit (Screening, Week 4, Week 12, and Week 24), including systolic BP (mmHg), diastolic BP (mmHg), and HR (bpm) by treatment group. Changes from baseline (Screening) to each post-baseline visit may be presented.

Clinically significant vital sign measurements at any visit may be presented in by-subject listings:
Systolic BP: >198 mmHg
Diastolic BP: >121 mmHg
HR: >130 bpm or <40 bpm

### Physical Examination

The number and percent of subjects with normal, abnormal not clinically significant (NCS), and abnormal clinically significant (CS) physical examination results may be summarized at each visit by body system and treatment group.

### Intraocular Pressure (IOP)

Observed values and change from baseline (Screening) in IOP may be summarized descriptively at each visit by treatment group. Separate summaries may be created for the study eye and the fellow eye.

### BCVA

Observed values and change from baseline (Screening) in BCVA (visual acuity score using letters read as the unit) may be summarized descriptively using counts and percentages for each treatment group at each visit by treatment group. Separate summaries may be created for the study eye and the fellow eye.

The count and percent of subjects with improvement or loss of ≥15, ≥10, and ≥5 letters in BCVA compared to baseline at each visit for the study eye and the fellow eye may also be summarized.

### Clinical Laboratory Tests

Observed values and change from baseline in clinical laboratory results (blood chemistry and hematology) may be summarized descriptively for each parameter and visit by treatment group. Lab values may be summarized using standard units. Hemoglobin A1c (HbA1c) values (if available) may be included in the hematology summary.

Laboratory results flagged as “critical” by the central laboratory may be considered clinically significant and may be presented in by-subject listings.

### Biomicroscopy and Ophthalmoscopy

Results from slit lamp biomicroscopy and dilated indirect ophthalmoscopy exams may be summarized at each visit by assessment type and treatment group. Abnormal NCS results from biomicroscopy and ophthalmoscopy exams may be presented in by-subject listings.

### Other Safety Measures

Urine pregnancy tests for women of childbearing potential may be conducted at each study visit and presented in by-subject listings.

### PK Analyses

PK data may be assessed using the PK Population, with subjects included in the treatment group they actually received, regardless of their randomized treatment. PK endpoints may be analyzed using non-linear mixed effect modeling, also called Population PK Analysis.

### EXAMPLES

The invention now being generally described, In be more readily understood by reference to the following examples, which are included merely for purposes of illustrating certain aspects and embodiments of the present invention, and are not intended to limit the invention.

### Example 1—Preventing the Progression of Diabetic Retinopathy

Ability of Compound 1 to prevent the progression of non-proliferative diabetic retinopathy (NPDR) and mild proliferative diabetic retinopathy (PDR) may be evaluated according to a clinical study in which Compound 1 was orally administered to patients suffering from non-proliferative diabetic retinopathy or mild proliferative diabetic retinopathy. Compound 1 has the chemical name (E)-2-((4,5-dimethoxy-2-methyl-3,6-dioxocyclohexa-1,4-dien-1-yl)methylene)undecanoic acid, and is depicted by the following chemical formula:

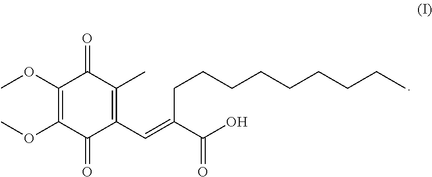 [SMILES: CCCCCCCCC/C(=C\C1=C(C)C(=O)C(OC)=C(OC)C1=O)C(=O)O]

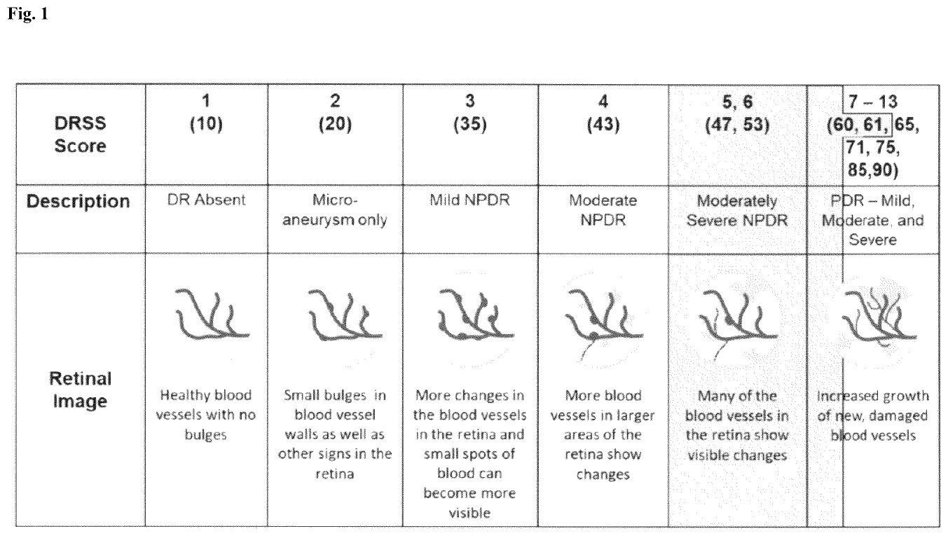

The study was configured as a placebo-controlled, double-masked, randomized, Phase 2 study in approximately 100 subjects with moderately severe to severe NPDR (diabetic retinopathy severity score [DRSS] Level 47 or 53, see FIG. 1) or mild PDR (DRSS Level 61), evaluating safety and efficacy following administration of Compound 1 twice daily for 24 weeks. The study was configured for a 1:1 randomization (placebo: Compound 1). Randomization was stratified by level of disease severity (NPDR or PDR). Subjects with mild PDR were capped at 20% for each arm. Efficacy evaluations at 12 and/or 24 weeks included DRSS, center-involved diabetic macular edema (DME), moderate PDR or PDR-related adverse events (AEs), best-corrected visual acuity (BCVA), and central subfield thickness (CST). Further experimental procedures and results are described below.

### Part I—Experimental Procedures

The study was performed according to the protocol outlined below. The total length of subject participation was approximately 26 weeks, with 5 clinic visits, 4 telephone safety calls, and one telephone call follow-up visit summarized below:
Screening Visit 1 (up to 7 days prior to Baseline Visit)
Qualification/Baseline Visit 2 (1 day)
Treatment-study period (24 weeks)
   Treatment Visit 4 (Week 4), Visit 6 (Week 12), and Visit 9 (Week 24)
   Telephone Safety Call Visit 3 (Week 1), Visit 5 (Week 8), Visit 7 (Week 16), and Visit 8 (Week 20)
Follow-up Phone Call Visit 10 (2 days)

Human subjects are screened for potential enrollment and, if qualified, enrolled in the study. Inclusion criteria and exclusion criteria for the study are set forth below. Human subjects can qualify in either eye. The eligible eye with the higher DRSS was designated as the study eye for the primary endpoint efficacy analysis. If the PDR cap is reached, the study eye may be an eye with the lower DRSS, if the other eye has mild PDR. If both eyes have the same DRSS, the eye with the worse BCVA was selected as the study eye. If the DRSS and BCVA are equivalent between both eyes, the study eye was the right eye.

### Inclusion Criteria

Males or non-pregnant females≥18 years of age.
At least one eye with diabetic retinopathy graded at least moderately severe to severe NPDR or mild PDR (corresponding to DRSS 47, 53, or 61, confirmed by a central reading center) in which panretinal laser photocoagulation (PRP) and intravitreal injections of an anti-VEGF agent can be safely deferred for ≥6 months in the opinion of the Investigator.
BCVA assessed by Early Treatment Diabetic Retinopathy Study (ETDRS) protocol letters score of ≥60 letters (Snellen equivalent≥20/63) in the study eye.
Sufficiently clear ocular media, adequate pupillary dilation, and fixation to permit quality fundus imaging in both eyes.
Able to cooperate sufficiently for ophthalmic visual function testing and anatomic assessment.
Body mass index (BMI) between 18 and 40 kg/m², inclusive.
Able and willing to give signed informed consent and follow study instructions.
Able to self-administer oral study medication or to have study medication administered by a caregiver throughout the study period.

### Exclusion Criteria—Ophthalmic

Retinopathy from causes other than diabetes.
Presence of center-involved diabetic macular edema (DME) defined as a central subfield thickness (CST)≥300 μm on SD-OCT or the presence of intra- or subretinal fluid within the central subfield. Center-involved DME in the fellow eye is allowed. Intravitreal injections of an anti-VEGF agent in the fellow eye does not exclude the subject.
Any prior treatment in the study eye with excluded concomitant medication/treatment:
   Focal or grid laser photocoagulation within the past year or PRP at any time.
   Systemic or intravitreal anti-VEGF agents within the last 6 months or likely, in the opinion of the Investigator, to require treatment during the course of the study.
   Intraocular steroids including triamcinolone and dexamethasone implant within the last 6 months.
   Fluocinolone implant within the last 3 years.
Clinically significant ocular disease in either eye as deemed by the Investigator to likely interfere with the study procedures and visual acuity measurements (e.g., cataract, pseudophakia without evidence of posterior capsular opacity, glaucoma, corneal edema, uveitis, severe keratoconjunctivitis sicca).
Presence of other macular or retinal vascular disease including age-related macular degeneration, pattern dystrophy, choroidal neovascularization of any cause, retinal vein occlusion, retinal artery occlusion in the study eye.
Presence of active vitreous hemorrhage that would prevent adequate clinical imaging in either eye.
History of retinal detachment or full-thickness macular hole in the study eye.
Uncontrolled glaucoma in either eye, defined as advanced cup-to-disc ratio>0.7 and intraocular pressure (IOP)>25 mmHg, with or without topical antihypertensive eye drops; treatment of ocular hypertension or controlled glaucoma are not criteria for exclusion.
Ocular incisional surgery including cataract surgery in the study eye within 3 months prior to Day 1.
Yttrium aluminum garnet (YAG) posterior capsulotomy in the study eye within the last 30 days.
Aphakia in the study eye.
Previous pars plana vitrectomy in the study eye.
Epiretinal membrane, posterior hyaloidal traction, and/or vitreomacular traction in the study eye as determined to be significant by the Investigator.
Active uveitis and/or vitritis in either eye.
History of idiopathic or autoimmune-associated uveitis in either eye.
Active infection in either eye including infectious conjunctivitis, keratitis, scleritis, or endophthalmitis.

### Exclusion Criteria—Systemic

Poorly controlled diabetes, defined as hemoglobin A1c (HbA1c)≥12.0% or <12.0% with uncontrolled diabetes mellitus or no HbA1c available.
Known to be immunocompromised or receiving immunosuppressive therapy.
Any disease or medical condition that in the opinion of the Investigator would prevent the subject from successfully participating in the study or which might confound the study results.
Clinically significant systemic disease (e.g., uncontrolled diabetes, myasthenia gravis, cancer, hepatic, renal, endocrine, or cardiovascular disorders) that might interfere with the study as deemed by the Investigator.
Estimated glomerular filtration rate (eGFR)<30 mL/min by Modification of Diet or Renal Disease (MDRD) or creatinine>4 mg/dL.
History of allergic reaction to investigational drug or any of its components.
Resting heart rate (HR) outside the specified range of 50-110 beats per minute at the Screening Visit. HR may be repeated only once if outside the specified range following at least a 5-minute rest period in the sitting position.
Hypertension with resting diastolic blood pressure (BP)>110 mmHg or systolic BP>180 mmHg at the Screening Visit. BP may be repeated only once if outside the specified range following at least a 5-minute rest period in the sitting position.
History of chronic liver disease or presence of elevated alanine aminotransferase (ALT) and aspartate aminotransferase (AST) consistent with such diagnosis (i.e., AST or ALT>2× upper limit of normal).
Participation in any investigational study within 30 days prior to Screening or planning to participate in any other investigational drug or device clinical trials within 30 days of study completion.
Women of childbearing potential who are pregnant, nursing, planning a pregnancy, or not using a medically acceptable form of birth control. All women of childbearing potential must have a negative urine pregnancy test result at the Visit 1/Screening examination and must intend to not become pregnant during the study.

Patients with DME in the fellow eye were eligible for enrollment into the study, however center-involved DME in the study eye is exclusionary.

Subjects were screened at Visit 1 and those successfully completing eligibility requirements were to return to site for their Qualification/Baseline Visit (Visit 2/Day 1) where they undergo a set of safety and lab test assessments and study medication was dispensed. Subjects were then return to site at Visit 4 (Week 4), Visit 6 (Week 12) and Visit 9 (Week 24) for safety and efficacy assessments. In between these site visits, subjects were contacted by telephone on Visit 3 (Week 1), Visit 5 (Week 8), Visit 7 (Week 16), and Visit 8 (Week 20) for a safety assessment to include AEs, concomitant medications, and drug accountability.

Study medication was dispensed initially at Visit 2 (Day 1) and then at Visit 4 (Week 4) and Visit 6 (Week 12) at the site. Subjects were to bring all unused study medication to each site visit for drug accountability. Study medication was collected at site during Visit 9 (Week 24). A Follow-up Phone Call was conducted one week after Visit 9 (Week 24) for AE and concomitant medication assessments.

At the Screening (Visit 1, Day −7 to −1):
A member of the study center staff interviews the individual as to their qualifications for participation in the study, and if the subject wishes to continue, the informed consent form is signed.
The start of Screening includes the assignment of a subject identification number, an explanation of the study, a medical and ophthalmic history, demographics, and a review of prior/concomitant medications.
Then the following were conducted: physical examination, measurement of HR/BP, and a urine pregnancy test for women of childbearing potential.
Subsequently, BCVA was measured and SD-OCT (for CST) and color fundus photographs (for DRSS) performed. DRSS eligibility was determined by a central reading center with 7-field or 4-wide field fundus photography. The central reading center also determines CST eligibility with SD-OCT. BCVA (distance) is measured with a Standard ETDRS illuminated chart (on wall or stand) at 4 m.
Then the following were conducted: assessments of blood chemistry and hematology, ophthalmic examination that includes biomicroscopy and direct or indirect ophthalmoscopy, IOP assessment, eGFR, and AEs.

If the subject meets all eligibility criteria (including DRSS and SD-OCT), then BCVA, DRSS, CST and other safety assessments performed at Screening are the baseline values, and the subject was asked to return for their Qualification Visit.

At the Qualification/Baseline (Visit 2, Day 1):
The subject was randomized into the study.
Study medication was dispensed in accordance with the subjects randomized treatment arm.
Blood samples were drawn pre-dose for baseline levels and on Visit 9 (Week 24) for exploratory biomarker processing (i.e., cytokine and Ref-1 levels) to evaluate pharmacodynamic properties.
Subjects were instructed to administer their study medication (Compound 1 or placebo) each day, with 3 tablets every morning and 2 tablets every evening. Study medication was to be taken at approximately the same time each day and may be taken with or without food.

Telephone safety calls were conducted for Visit 3 (Week 1±2 Days), Visit 5 (Week 8±2 Days), Visit 7 (Week 16±2 Days), and Visit 8 (Week 20±2 Days). The safety assessment included review of drug compliance, concomitant medications, AEs, and urine pregnancy test at home (only for women of childbearing potential).

At the First Treatment Visit (Visit 4, Week 4±2 Days), subjects were to return to the site for the following series of safety and efficacy assessments: drug accountability, concomitant medications, urine pregnancy test (only for women of childbearing potential), R/BP/vital signs, BCVA (ETDRS), biomicroscopy, ophthalmoscopy, IOP, and AEs. Following the completion of the assessments, used medication kits were collected for accountability (by counting the number of unused tablets), and new study medication kits dispensed.

At the Second Treatment Visit (Visit 6, Week 12±2 Days), subjects were to return to the site for the following series of safety and efficacy assessments: drug accountability, concomitant medications, urine pregnancy test (only for women of childbearing potential), HR/BP/vital signs, BCVA (ETDRS), DRSS, CST (SD-OCT), blood draw for PK, blood chemistry, blood hematology, biomicroscopy, ophthalmoscopy, IOP, eGFR, and AEs. Following the completion of the assessments, used medication kits were collected for accountability (by counting the number of unused tablets), and new study medication kits dispensed.

At the Third Treatment Visit (Visit 9, Week 24±2 Days), subjects were to return to the site for the following series of safety and efficacy assessments: drug accountability, concomitant medications, urine pregnancy test (only for women of childbearing potential), physical examination, HR/BP/vital signs, BCVA (ETDRS), DRSS, CST (SD-OCT), blood chemistry, blood hematology, biomicroscopy, ophthalmoscopy, IOP, eGFR, AEs, and blood draw for exploratory biomarkers (ELISA, cytokine panel, comprehensive metabolic panel). Visit 9 (Week 24) was the end of the treatment period. Study medication was returned for accountability (by counting the number of unused tablets), and no further study medication dispensed.

A telephone follow-up call was conducted for Visit 10 (Week 25±2 Days) to evaluate concomitant medications and AEs.

Study subjects received study medication as set forth in Table 1 according to the Treatment Group to which the subject is assigned. Subjects were instructed to take study medication at approximately the same time each day, and the medication may be taken with or without food. Study medication is listed in Table 2.

TABLE 1
Treatment Groups

| Treatment Group | Study Medication and Administration Protocol |
|---|---|
| 1 | Five tablets each containing 120 mg of Compound 1 |
| | by mouth each day, with 3 tablets every morning |
| | and 2 tablets every evening for 24 weeks, for |
| | subjects randomized to active treatment. |
| 2 | Five Placebo tablets by mouth each day, with |
| | 3 tablets every morning and 2 tablets every evening |
| | for 24 weeks, for subjects randomized to placebo. |

TABLE 2
Study Medication

| Study Medication | Composition of Study Medication |
|---|---|
| 120-mg Tablets | The immediate-release tablets consist |
| of Compound 1 | of a mixture of intragranular components |
| | to which an extragranular layer is |
| | applied, all being compressed as circular |
| | disks and film-coated with Opadry Yellow |
| | (pale-orange to light-yellow tablets). |
| | The intragranular components are: |
| | Compound 1 (120 mg per tablet) |
| | Lactose monohydrate |
| | Microcrystalline cellulose NF (Avicel |
| | PH101) |
| | Starch 1500 |
| | Sodium carboxymethylcellulose (Aqualon |
| | 7MF PH) |
| | Methylcellulose A15LV USP. |
| | The extragranular components are: |
| | Microcrystalline cellulose NF (Avicel |
| | PH102) |
| | Sodium carboxymethylcellulose (Aqualon |
| | 7MF PH) |
| | Magnesium stearate. |
| Placebo Tablets | Placebo tablets are immediate release, |
| | identical in shape and color to the tablets |
| | containing 120 mg of Compound 1, except for |
| | the absence of the active pharmaceutical |
| | ingredient. |

Any subject was permitted to voluntarily withdraw from the study at any time without prejudice. A non-completing subject was defined as one who exited the study by their own volition or at the discretion of the Investigator and/or the Medical Monitor prior to completing all of the study procedures required in the protocol.

If a subject considers discontinuing from the study due to an AE, the Investigator was permitted to offer a dose reduction from 600 mg to 480 mg per day as an alternative (2 tablets every morning and 2 tablets every evening).

### Evaluation of Efficacy and Safety Endpoints and Measurement Procedures

Efficacy endpoints included:
the percent of subjects with a ≥2-step improvement in DRS S in the study eye at Week 24.
Percent of subjects with an improvement or worsening in DRSS (see FIG. 1) of ≥1, ≥2, ≥3, and ≥4 steps at Week 12 and Week 24,
Mean change from baseline in DRSS at Week 24,
Percent of subjects not developing center-involved DME or moderate PDR or PDR-related AEs during the study at Week 12 and Week 24,
Mean change from baseline in BCVA at Week 24, and
Mean change from baseline in CST.

Primary and secondary endpoints were evaluated in the study eyes, fellow eyes, all qualified eyes (study eyes and fellow eyes that meet all study eye eligibility criteria), and either eye (i.e., best response). All of the efficacy endpoints are analyzed by modified intention-to-treat (mITT) and per protocol (PP) populations. Other subpopulations may be identified and analyzed.

Exploratory efficacy endpoints include:
Relationship between Compound 1 in plasma measured at Week 12, and change from baseline in DRSS in the study eye at Week 12 and Week 24,
Mean change from baseline in plasma Ref-1 levels at Week 24,
Mean change from baseline in cytokine levels at Week 24,
Relationship between Ref-1 levels in plasma and change from baseline in DRSS in the study eye at Week 24, and
Relationship between cytokines levels in plasma and change from baseline in DRSS in the study eye at Week 24.

Measurements were determined as follows, where every effort will be made to have the same person perform the measurements at all timepoints and at all visits:
DRSS will be measured with 7-field or 4-wide field color fundus photographs,
CST will be measured using SD-OCT, and
BCVA will be measured by Standard ETDRS chart at 4 m (letters).

For pharmacokinetics analysis, at Visit 6 (Week 12±2 days) pre-morning dose and 3 hours post-morning dose, blood samples were collected to establish drug levels of Compound 1 from approximately 25 to 30 subjects at a subset of clinical sites. These subjects were instructed to delay their morning study medication dose on the day of this visit so that they will take their study medication at the site. Five mL of blood was drawn immediately pre-dosing to establish a steady-state drug level. A second 5-mL sample was drawn 3 hours later to establish the Cₘₐₓ drug level. Analysis of plasma samples for Compound 1 concentration determinations was performed by a central PK laboratory using a validated liquid chromatography-mass spectrometry and liquid chromatography-tandem mass spectrometry (LC-MS/MS) method.

Safety endpoints include:
Incidence and severity of systemic and ocular AEs,
Change from baseline in body system assessments,
Change from baseline in vital sign measurements,
Change from baseline in clinical laboratory assay results (blood chemistry, hematology),
Change from baseline in intraocular pressure (IOP),
Change from baseline in slit-lamp examination parameters,
Change from baseline in dilated funduscopic examination parameters,
Percent of subjects with a decrease of ≥10 letters in BCVA compared to baseline at Week 12 and Week 24,
Percent of subjects progressing to center-involved DME (eligible for rescue treatment at Week 12 and Week 24),
Percent of subjects with a worsening in DRSS of ≥2 steps in the study eye at Week 12 and Week 24,
Percent of subjects developing anterior segment neovascularization at Week 12 and Week 24,
Percent of subjects rescued (intravitreal anti-VEGF injection, laser PRP, focal/grid laser treatment, or surgery [vitrectomy]) at the discretion of the Investigator at Week 12 and Week 24, and
Change from baseline in estimated glomerular filtration rate (eGFR) at Week 12 and Week 24.

### Evaluation of Efficacy and Safety General Analysis Procedures

Analysis populations included:
Modified Intention-to-Treat (mITT): The mITT includes all randomized subjects who received at least one dose of study treatment and at least one post-dose efficacy measurement. The mITT will be used to analyze efficacy endpoints.
Per Protocol Population (PP): The PP population includes all subjects in the mITT who have missed less than 20% of expected doses and do not have any major protocol deviations considered to have significant impact on treatment outcome. The PP population will be used for primary endpoint analysis and to analyze efficacy endpoints.
All Randomized Population (ARP): The ARP includes all randomized subjects. This population is also known as the Intent-to-Treat (ITT) population. The ARP will be used in confirmatory efficacy analyses.
Safety Population (SP): The SP includes all randomized subjects who have received at least one dose of study treatment. The SP was used to summarize safety variables.

### Part II—Results

Provided below are results showing ability of Compound 1 to prevent the progression of diabetic retinopathy and results showing the safety of Compound 1.

### Efficacy Against Diabetic Retinopathy

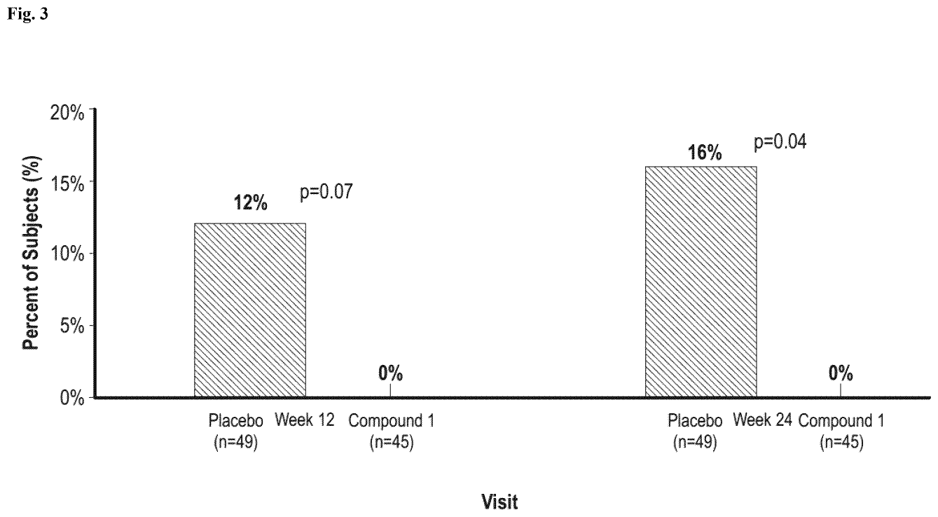

FIG. 3 shows results from the trial that are the number of patients that experienced a three or more step worsening in the patients' binocular Diabetic Retinopathy Severity Scale Score at week 12 and at week 24 for patients that received placebo and for patients that received Compound 1. The results in FIG. 3 demonstrate that Compound 1 was effective in preventing the progression of diabetic retinopathy at each of weeks 12 and 24.

### Safety

Provided below is data on safety on Compound 1 orally administered to patients according to the study protocol described herein. The data is from one-hundred patients that were enrolled in the trial. Per the study protocol described above, patients enrolled in the study were randomized 1:1 for receipt of placebo versus Compound 1. The safety data below is the combined results from patients that received placebo and those received Compound 1. The results show that Compound 1 administered according to the study protocol had a good safety profile in the patient population enrolled in this study.

Table 3 below provides results from analysis of concentration of alanine aminotransferase in subjects' blood.

TABLE 3

| | Number of Subjects for | |
|---|---|---|
| | Which Have Data | Mean Concentration |
| | for Mean Concentration | of Alanine |
| | of Alanine | Aminotransferase in |
| Time | Aminotransferase in | Subjects' |
| Point | Subjects' Blood | Blood (IU/L) |
| Baseline | 97 | 22.4 |
| Week 12 | 53 | 21.6 |
| Week 24 | 24 | 20.4 |

Comparison of data available for 50 subjects at week 12 showed a change of −0.8 IU/L in mean concentration of alanine aminotransferase in subjects' blood relative to the mean concentration of alanine aminotransferase in subjects' blood at Baseline. This corresponds to a 3.6% reduction in mean concentration of alanine aminotransferase in subjects' blood at week 12 relative to the mean concentration of alanine aminotransferase in subjects' blood at Baseline.

Comparison of data available for 22 subjects at week 24 showed a change of −2.1 IU/L in mean concentration of alanine aminotransferase in subjects' blood relative to the mean concentration of alanine aminotransferase in subjects' blood at Baseline. This corresponds to a 9.4% reduction in mean concentration of alanine aminotransferase in subjects' blood at week 24 relative to the mean concentration of alanine aminotransferase in subjects' blood at Baseline.

Table 4 below provides results from analysis of concentration of aspartate aminotransferase in subjects' blood.

TABLE 4

| | Number of Subjects for | |
|---|---|---|
| | Which Have Data | Mean Concentration |
| | for Mean Concentration | of Aspartate |
| | of Aspartate | Aminotransferase in |
| Time | Aminotransferase in | Subjects' Blood |
| Point | Subjects' Blood | (IU/L) |
| Baseline | 97 | 19.7 |
| Week 12 | 53 | 19.2 |
| Week 24 | 24 | 17.5 |

Comparison of data available for 50 subjects at week 12 showed a change of −0.5 IU/L in mean concentration of aspartate aminotransferase in subjects' blood relative to the mean concentration of aspartate aminotransferase in subjects' blood at Baseline. This corresponds to a 3% reduction in mean concentration of aspartate aminotransferase in subjects' blood at week 12 relative to the mean concentration of aspartate aminotransferase in subjects' blood at Baseline.

Comparison of data available for 22 subjects at week 24 showed a change of −1.8 IU/L in mean concentration of aspartate aminotransferase in subjects' blood relative to the mean concentration of aspartate aminotransferase in subjects' blood at Baseline. This corresponds to a 9% reduction in mean concentration of aspartate aminotransferase in subjects' blood at week 24 relative to the mean concentration of aspartate aminotransferase in subjects' blood at Baseline.

Table 5 below provides results from analysis of glomerular filtration rate of subjects.

TABLE 5

| | Number of Subjects for | |
|---|---|---|
| | Which Have Data | Mean Glomerular |
| Time | for Mean Glomerular | Filtration Rate |
| Point | Filtration Rate | (mL/min/1.73 m²) |
| Baseline | 94 | 92.53 |
| Week 12 | 52 | 83.85 |
| Week 24 | 24 | 77.7 |

Comparison of data available for 47 subjects at week 12 showed a change of −2.24 mL/min/1.73 m² in mean glomerular filtrate rate in subjects relative to the mean glomerular filtrate rate in subjects at Baseline. This corresponds to a 2% reduction in mean glomerular filtrate rate in subjects at week 12 relative to the mean glomerular filtrate rate in subjects at Baseline.

Comparison of data available for 21 subjects at week 24 showed a change of −9.84 mL/min/1.73 m² in mean glomerular filtrate rate in subjects relative to the mean glomerular filtrate rate in subjects at Baseline. This corresponds to a 11% reduction in mean glomerular filtrate rate in subjects at week 24 relative to the mean glomerular filtrate rate in subjects at Baseline.

Table 6 below provides results from analysis of heart rate of subjects.

TABLE 6

| | Number of Subjects for | |
|---|---|---|
| Time | Which Have Data | Mean Heart |
| Point | for Mean Heart | Rate (beats/min) |
| Baseline | 99 | 76.7 |
| Week 12 | 62 | 76.3 |
| Week 24 | 27 | 80.7 |

Comparison of data available for 62 subjects at week 12 showed a change of +0.9 beats/min in mean heart rate in subjects relative to the mean heart rate in subjects at Baseline. This corresponds to a 1% increase in mean heart rate in subjects at week 12 relative to the mean heart rate in subjects at Baseline.

Comparison of data available for 27 subjects at week 24 showed a change of +4.3 beats/min in mean heart rate in subjects relative to the mean heart rate in subjects at Baseline. This corresponds to a 6% increase in mean heart rate in subjects at week 24 relative to the mean heart rate in subjects at Baseline.

Table 7 below provides results from analysis of systolic blood pressure of subjects.

TABLE 7

| | Number of Subjects for | |
|---|---|---|
| | Which Have Data | Mean Systolic |
| Time | for Mean Systolic | Blood Pressure |
| Point | Blood Pressure | (mmHg) |
| Baseline | 99 | 136.9 |
| Week 12 | 62 | 132 |
| Week 24 | 27 | 139.7 |

Comparison of data available for 62 subjects at week 12 showed a change of −4.7 mmHg in mean systolic blood pressure in subjects relative to the mean systolic blood pressure in subjects at Baseline. This corresponds to a 3% reduction in mean systolic blood pressure in subjects at week 12 relative to the mean systolic blood pressure in subjects at Baseline.

Comparison of data available for 27 subjects at week 24 showed a change of −0.7 mmHg in mean systolic blood pressure in subjects relative to the mean systolic blood pressure in subjects at Baseline. This corresponds to a 1% reduction in mean systolic blood pressure in subjects at week 24 relative to the mean systolic blood pressure in subjects at Baseline.

Table 8 below provides results from analysis of diastolic blood pressure of subjects.

TABLE 8

| | Number of Subjects for | |
|---|---|---|
| | Which Have Data | Mean Diastolic |
| Time | for Mean Diastolic | Blood Pressure |
| Point | Blood Pressure | (mmHg) |
| Baseline | 99 | 80.3 |
| Week 12 | 62 | 77.1 |
| Week 24 | 27 | 80.4 |

Comparison of data available for 62 subjects at week 12 showed a change of −2.8 mmHg in mean diastolic blood pressure in subjects relative to the mean diastolic blood pressure in subjects at Baseline. This corresponds to a 3% reduction in mean diastolic blood pressure in subjects at week 12 relative to the mean diastolic blood pressure in subjects at Baseline.

Comparison of data available for 27 subjects at week 24 showed a change of +1 mmHg in mean diastolic blood pressure in subjects relative to the mean diastolic blood pressure in subjects at Baseline. This corresponds to a 1% increase in mean diastolic blood pressure in subjects at week 24 relative to the mean diastolic blood pressure in subjects at Baseline.

Table 9 below provides a summary of occurrence of adverse events across 100 subjects enrolled in the study, which is the combined results from patients that received placebo and those that received Compound 1. Of these 100 subjects, 41 subjects reported at least one treatment-emergent adverse event. A total of 83 treatment-emergent adverse events were observed from the 100 subject enrolled in the study. A subject reporting more than one treatment-emergent adverse event is only counted once within the System Organ Class in Table 9.

TABLE 9

| | No. of Subjects That |
|---|---|
| | Experienced a |
| | Treatment-Emergent |
| Category of Adverse Event | Adverse the Category |
| Eye disorder | 9 |
| Anterior^{[1]} | 5 |
| Posterior^{[1]} | 9 |
| Gastrointestinal disorder | 9 |
| Infection or infestation | 9 |
| Nervous system disorder | 7 |
| Skin and subcutaneous | 7 |
| tissue disorder | |
| Musculoskeletal and connective | 5 |
| tissue disorder | |
| Not coded | 4 |
| Investigation | 3 |
| Psychiatric disorder | 2 |
| Respiratory, thoracic, or | 2 |
| mediastinal disorder | |
| Blood or lymphatic system | 1 |
| disorder | |
| Cardiac disorder | 1 |
| Ear or labyrinth disorder | 1 |
| General disorders or | 1 |
| administration site condition | |
| Hepatobiliary disorder | 1 |
| Injury, poisoning, or | 1 |
| procedural complication | |
| Metabolism or nutrition | 1 |
| disorder | |
| Reproductive system or | 1 |
| breast disorder | |
| Vascular disorder | 1 |

^{[1]}Bilateral ocular events are counted twice, i.e., once for each eye.

### Example 2—Additional Study on Preventing the Progression of Diabetic Retinopathy

Ability of Compound 1 or a salt thereof to prevent the progression of non-proliferative diabetic retinopathy (NPDR) may be evaluated according to a clinical study in which Compound 1 or a salt thereof is orally administered to patients suffering from non-proliferative diabetic retinopathy, such as moderate to severe non-proliferative diabetic retinopathy. Compound 1 has the chemical name (E)-2-((4,5-dimethoxy-2-methyl-3,6-dioxocyclohexa-1,4-dien-1-yl)methylene)undecanoic acid, and is depicted by the following chemical formula:

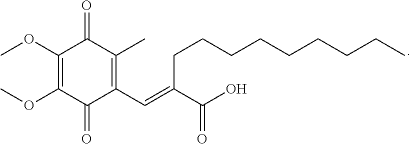 [SMILES: CCCCCCCCC/C(=C\C1=C(C)C(=O)C(OC)=C(OC)C1=O)C(=O)O]

The term “Active Compound” used in description of this clinical study refers to Compound 1 or a salt thereof.

The study may be configured as a placebo-controlled, double-masked clinical study in human subjects with moderate to severe NPDR evaluating safety and efficacy following administration of Compound 1 or salt thereof, such as twice daily. In certain embodiments, the subject may have a diabetic retinopathy severity score [DRSS] Level of 47 or 53, see FIG. 1. The study may be configured for a 1:1 randomization (placebo: Compound 1 or salt thereof). Efficacy evaluations may include DRSS, center-involved diabetic macular edema (DME), moderate PDR or PDR-related adverse events (AEs), best-corrected visual acuity (BCVA), central subfield thickness (CST), and other features. Further experimental procedures are described below.

In a more particular design, the clinical study may be configured as a randomized, placebo-controlled, double-masked study to evaluate the efficacy and safety of Compound 1 or a salt thereof (e.g., a calcium salt of Compound 1) orally administered to human subjects suffering from moderate to severe NPDR (DRSS level 43-53) in both eyes and with level 47 or 53 in at least 1 eye. The eye with the highest DRSS will be designated as the study eye for the primary endpoint efficacy analysis. If eyes have the same DRSS, the eye with the worse BCVA will be selected as the study eye. If the DRSS and BCVA are equivalent between eyes, the study eye will be the right eye. Compound 1 or a salt thereof is orally administered to subjects in an amount of 300 mg BID for 96 weeks (24 months). More generally, subjects will take two 300 mg tablets ((i) placebo or (ii) Compound or a salt thereof) orally each day (1 tablet every morning and 1 tablet every evening) for 96 weeks. Study medication is to be taken at approximately the same time each day and may be taken with or without food. All subjects should be instructed on the importance of following the dosing regimen (1 tablet BID. On Day 1, subjects will take their first dose of study medication at the study site and they will continue BID dosing for 96 weeks. If a subject is considering withdrawing from the study due to an AE, a dose reduction from 600 mg/day to 300 mg/day may be used as an alternative; to be taken preferably as an evening dose.

Eligible subjects participating in the study will be randomized 1:1 (placebo: Compound 1 or a salt thereof). Randomization will be stratified by baseline level of disease severity in the worse eye (DRSS level 47 or 53). A primary outcome measure of the study is a between-group comparison (placebo: Compound 1 or a salt thereof) of the percent of subjects with a ≥3-step worsening from baseline on the DRSS person-level scale at Week 48 (12 months).

Subjects may be evaluated for efficacy using color fundus photography (FP) for DRSS determination, ETDRS BCVA, spectral-domain optical coherence tomography (SD-OCT), UWF-FA, optical coherence tomography angiography (OCT-A), and RETeval ERG. Subjects may be evaluated for ocular and systemic safety using ophthalmic exams and laboratory assessments through Week 96 (End of Treatment [EOT]). Subject HRQOL may be measured using the 25-Item National Eye Institute Visual Function Questionnaire (NEI-VFQ-25) and the Medical Outcomes Study 12-Item Short Form Health Survey (SF-12).

Subjects who develop PDR, anterior-segment neovascularization (ASNV), or CI-DME with loss of ETDRS BCVA in either eye will qualify for rescue treatment. Subjects rescued for ASNV or CI-DME will discontinue study medication and be withdrawn from the study following the first instance of treatment for ASNV or CI-DME; subjects rescued for PDR may remain on their study medication and continue in the study.

Total length of individual subject participation may be approximately 100 weeks, with 12 clinic visits, 8 telephone safety calls, and 1 telephone follow-up call summarized below:
Screening Visit 1 (up to 28 days prior to the Baseline Visit).
Baseline Visit 2 (1 day).
Treatment period (96 weeks).
   Treatment Visit 4 (Week 4), Treatment Visit 5 (Week 8), Treatment Visit 6 (Week 12), Visit 8 (Week 24), Visit 10 (Week 36), Visit 12 (Week 48), Visit 14 (Week 60), Visit 16 (Week 72), Visit 18 (Week 84), Visit 20 (Week 96/EOT).
   Safety Phone Call Visit 3 (Week 1), Visit 7 (Week 18), Visit 9 (Week 30), Visit 11 (Week 42), Visit 13 (Week 54), Visit 15 (Week 66), Visit 17 (Week 78), Visit 19 (Week 90).
Follow-Up Phone Call Visit 21 (1 week after EOT Visit; 3-day window).

In preparation for conducting the study, human subjects are screened for potential enrollment and, if qualified, enrolled in the study. Exemplary inclusion criteria and exclusion criteria for the study are set forth below.

### Exemplary Inclusion Criteria

1. Males or females≥18 years of age.
2. Body mass index between 18 and 40 kg/m², inclusive.
3. Diagnosis of diabetes mellitus (type 1 or type 2). Any one of the following will be considered to be sufficient evidence that diabetes is present:
   a. Current regular use of insulin for the treatment of diabetes for at least the 3 months prior to Screening.
   b. Current regular use of oral anti-hyperglycemia agents for the treatment of diabetes for at least the 3 months prior to Screening.
   c. Documented diabetes (type 1 or type 2) as classified by the American Diabetes Association and/or World Health Organization criteria.
4. Both eyes with at least moderate to severe NPDR (corresponding to DRSS level 43-53), and with level 47 or 53 in at least 1 eye, confirmed by the central reading center.
5. Early Treatment Diabetic Retinopathy Study BCVA score of ≥70 letters (Snellen equivalent 20/40 or better) in both eyes.
6. An ERG DR severity score of ≥23.5 in the study eye.
7. Sufficiently clear ocular media, adequate pupillary dilation, and adequate fixation to permit quality fundus imaging in both eyes.
8. Able to cooperate sufficiently for ophthalmic visual function testing and anatomic assessment.
9. Able and willing to give signed informed consent and follow study instructions.
10. Able to compliantly self-administer oral study medication or to have study medication administered by a caregiver BID for 24 consecutive months.

### Exemplary Exclusion Criteria—Ophthalmic

1. Retinopathy from causes other than diabetes.
2. Evidence of retinal neovascularization on dilated ophthalmic exam or central reading center grading of UWF-FA, including neovascularization outside of the 7 modified ETDRS fields.
3. Presence of CI-DME, defined as CST≥290 μm in women or ≥305 μm in men using the Zeiss Cirrus SD-OCT or CST≥305 μm in women or ≥320 μm in men using the Heidelberg Spectralis SD-OCT, confirmed by the central reading center.
4. Prior treatment with:
   a. Panretinal laser photocoagulation.
   b. Pars plana vitrectomy.
   c. Yttrium aluminum garnet (YAG) posterior capsulotomy within 90 days prior to Screening.
   d. Ocular incisional surgery, including cataract surgery within the last 3 months.
   e. History of trabeculectomy or insertion of a drainage device (excluding microinvasive glaucoma surgery) for the treatment of glaucoma.
   f. History of focal or grid laser photocoagulation within the macula.
   g. Systemic or intravitreal anti-VEGF agents within the last 12 months.
   h. Intraocular steroids, including triamcinolone and dexamethasone implant within the last 12 months.
   i. Fluocinolone implant within the last 3 years.
5. Clinically significant ocular disease as deemed by the Investigator to likely interfere with the study procedures and VA measurements (e.g., cataract, pseudophakia without evidence of posterior capsular opacity, glaucoma, corneal edema, uveitis, severe keratoconjunctivitis sicca).
6. Presence of other macular or retinal vascular disease, including age-related macular degeneration, pattern dystrophy, choroidal neovascularization from any cause, retinal vein occlusion, and/or retinal artery occlusion.
7. History of retinal detachment or full-thickness or lamellar macular hole.
8. Uncontrolled glaucoma, defined as advanced cup-to-disc ratio>0.7 or intraocular pressure (IOP)>25 mmHg, with or without topical antihypertensive eye drops; treatment of ocular hypertension or controlled glaucoma are not criteria for exclusion. Eyes on more than 3 IOP-lowering medications should be excluded.
9. Aphakia.
10. Epiretinal membrane, posterior hyaloidal traction, and/or vitreomacular traction if determined by the Investigator or reading center to be significantly affecting vision.
11. Active uveitis and/or vitritis.
12. History of idiopathic or autoimmune-associated uveitis.
13. Active infection, including infectious conjunctivitis, keratitis, scleritis, or endophthalmitis.
14. Planned cataract surgery during conduct of trial.

### Exemplary Exclusion Criteria—Systemic

1. Hemoglobin A1c≥12.0% at the Screening Visit.
2. Change in medication regimen for control of glucose within the past 3 months.
3. Initiation of treatment with a glucagon-like peptide 1 (GLP-1) agonist or sodium-glucose transport protein 2 (SGLT-2) antagonist within the past 6 months. Treatment with these agents should not be initiated while the subject is participating in the study.
4. Treatment with a thiazolidinedione (e.g., rosiglitazone or pioglitazone).
5. History of bariatric surgery.
6. Known to be immunocompromised or receiving immunosuppressive therapy.
7. Any clinically significant systemic disease or medical condition (e.g., uncontrolled diabetes, myasthenia gravis, currently receiving systemic treatment for cancer, or hepatic, renal, endocrine, or cardiovascular disorders) that in the opinion of the investigator would prevent the subject from successfully participating in the study or which might confound the study results.
8. Estimated glomerular filtration rate (eGFR)<30 mL/min by Modification of Diet in Renal Disease (MDRD) or creatinine>4 mg/dL or at risk to require dialysis within the next 12 months.
9. Hypertension with resting diastolic blood pressure (BP)>110 mmHg or systolic BP>180 mmHg at the Screening Visit. Blood pressure may be repeated once if outside the specified range following at least a 5-minute rest period in the sitting position.
10. History of chronic kidney or liver disease or presence of elevated alanine aminotransferase (ALT) or aspartate aminotransferase (AST) consistent with such diagnosis (i.e., AST or ALT>2× upper limit of normal).
11. Females of childbearing potential who are pregnant, breastfeeding, intending to become pregnant within the next 24 months, or unable or unwilling to use an acceptable method of contraception.
12. Non-vasectomized male subjects who are unable or unwilling to use an acceptable method of contraception.
13. History of allergic reaction to investigational drug or any of its components.
14. Planned or ongoing treatment with gemfibrozil (Lopid).
15. Allergy to fluorescein or unable to undergo fluorescein angiography.

### Reduction or Discontinuation of Study Medication

Study medication may be discontinued for the following reasons:
Adverse events: AEs include clinically significant laboratory abnormalities and intercurrent diseases reported by the subject or observed by the investigator with documentation on the case report form (CRF).
Death: If a subject dies, the AE that caused the death should be documented on the CRF and be noted as serious and fatal.
Disallowed concurrent medication: Any medication not allowed by the protocol would be a protocol violation.
Lack of efficacy: A subject may elect to discontinue study medication for a perceived lack of efficacy.
Investigator decision: A subject may be discontinued from study medication for reasons other than those bulleted previously if the investigator thinks it is not in the best interest of the subject to continue on study medication.
Pregnancy: A subject may be discontinued from study medication if pregnancy occurs while on study.
Other: If there is any other reason for discontinuation from study medication, this should be noted on the CRF.

If a subject is considering withdrawing from the study due to an AE, a dose reduction from 600 mg/day to 300 mg/day may be used as an alternative; to be taken preferably as an evening dose.

### A. Reasons for Withdrawal From Study

Subject withdraws consent.
Subject is lost to follow-up.
Investigator's decision.
Subject withdraws from the study for other reason.

### B. Entire Study Terminated

The entire study may be terminated.

### C. Actions After Discontinuation of Study Medication

All subjects who discontinue study medication due to a report of an AE must be followed up and provided appropriate medical care until their signs and symptoms have remitted or stabilized or until abnormal laboratory findings have returned to acceptable or pre-study limits.

### D. Actions After Subject Withdrawal From Study

For any subject who chooses to withdraw consent or who is non-compliant, every possible effort should be made by the investigator to ensure there is a final follow-up telephone call that includes assessments for AEs and concomitant medications.

### E. Completed Study

The study is completed when all randomized subjects have completed the study, all CRFs have been completed, and all CRF data are entered into the database.

### Initiation of Rescue Treatment for Diabetic Retinopathy Progression in Either Eye

If there is any risk to the eye(s) of the subject, an appropriate rescue treatment for DR progression will be administered. Eyes may be rescued if there is:
Moderate or severe active PDR and/or development of ASNV.
Progression to clinically significant CI-DME (increase of ≥75 μm from baseline in CST), with a ≥10-letter loss of ETDRS BCVA from baseline or a 5- to 9-letter loss of ETDRS BCVA from baseline on 2 successive study visits that is attributable to CI-DME.
Any finding that, in the judgment of the investigator, requires rescue treatment.

Prior to initiating the first rescue treatment only, ETDRS BCVA, color FP, SD-OCT, UWF-FA, OCT-A (if available), ERG (if available), slit lamp biomicroscopy, dilated indirect ophthalmoscopy, and IOP study procedures are to be performed, and the HRQOL questionnaires completed by the subject. These assessments may be performed as an Unscheduled Visit.

If rescue treatment is given for PDR, the subject may remain on their study medication and continue in the study and complete all remaining study visits per the visit schedule. If rescue treatment is given for ASNV, CI-DME, or at the discretion of the investigator, the subject will discontinue study medication and be withdrawn from the study following receipt of rescue treatment.

### Evaluation of Efficacy and Safety Endpoints and Measurement Procedures

The primary efficacy endpoint is the percent of subjects with a ≥3-step worsening from baseline on the DRSS person-level scale at Week 48.

Secondary efficacy endpoints will include:
Percent of subjects with an improvement or worsening on the DRSS person-level scale of ≥1, ≥2, ≥3, and ≥4 steps at Weeks 12, 24, 48, 72, and 96.
Mean change from baseline on the DRSS person-level scale at Weeks 12, 24, 48, 72, and 96.
Percent of subjects with a ≥3-step worsening from baseline on the binocular DRSS (sum of change of DRSS from baseline in OD and OS) at Weeks 48 and 96.
Percent of subjects developing CI-DME (defined as an increase in CST≥75 μm) in either eye during the treatment period.
Percent of subjects developing CI-DME in both eyes during the treatment period.
Percent of subjects developing PDR or vitreous hemorrhage or requiring vitrectomy or PRP in either eye during the treatment period.
Percent of subjects developing PDR or vitreous hemorrhage or requiring vitrectomy or PRP in both eyes during the treatment period.
Percent of subjects developing vision-threatening complications in either eye, including CI-DME, PDR and/or vitreous hemorrhage, and the need for rescue treatment with intravitreal anti-VEGF, PRP, or vitrectomy.
Percent of subjects developing vision-threatening complications in both eyes, including CI-DME, PDR and/or vitreous hemorrhage, and the need for rescue treatment with intravitreal anti-VEGF, PRP, or vitrectomy.
Time to development of CI-DME in either eye.
Time to development of PDR or PDR-related AEs in either eye.
Time to first rescue treatment in either eye.
Percent of subjects with a loss or gain of ≥5, ≥10, and ≥15 letters in ETDRS BCVA in either eye at Weeks 24, 48, 72, and 96.
Mean change from baseline in ETDRS BCVA in OD and OS at Weeks 24, 48, 72, and 96.
Percent of subjects with an increase or decrease of ≥50, ≥75, and ≥100 μm in CST in either eye at Weeks 24, 48, 72, and 96.
Mean change from baseline in CST in OD and OS at Weeks 24, 48, 72, and 96.

Primary and secondary endpoints will be evaluated in the study eyes and fellow eyes. All of the efficacy endpoints will be analyzed by the mITT Population and selected efficacy endpoints will be analyzed by the Per Protocol (PP) Population. Other subpopulations may be identified and analyzed.

### Exploratory Efficacy Endpoints:

Mean change from baseline in estimated glomerular filtration rate (eGFR) at Weeks 24, 48, 72, and 96.
Mean change from baseline in urine albumin-creatinine ratio (UACR) at Weeks 24, 48, 72, and 96.
Mean change from baseline in UACR in subjects with baseline UACR≥300 at Weeks 24, 48, 72, and 96.
Mean change from baseline in total pathologic macular fluid, including intraretinal and subretinal fluid on OCT in OD and OS at Weeks 24, 48, 72, and 96.
Mean change from baseline in macular vessel density on OCT-A (if available) in OD and OS at Weeks 24, 48, 72, and 96.
Mean change from baseline in retinal vascular leakage on UWF-FA in OD and OS at Weeks 24, 48, 72, and 96.
Mean change from baseline in macular and peripheral perfusion index on UWF-FA in OD and OS at Weeks 24, 48, 72, and 96.
Mean change from baseline DRSS as a function of baseline non-perfusion index in OD and OS at Weeks 24, 48, 72, and 96.
Mean change from baseline in ERG DR score in OD and OS at Weeks 24, 48, 72, and 96.
Mean change from baseline in area under the log contrast sensitivity function (AULCSF) (if available) in OD and OS at Weeks 24, 48, 72, and 96.
Mean change from baseline in NEI-VFQ-25 and SF-12 total score and sub-scale scores at Weeks 24, 48, 72, and 96.
Mean change from baseline in cataract severity.

### Measurements:

Diabetic Retinopathy Severity Scale will be measured with 7-field, 4-widefield, or ultra-widefield color fundus photographs.
Central subfield thickness will be measured using SD-OCT.
Best-corrected visual acuity (VA) will be measured by Standard ETDRS chart at 4 m (letters).
Vascular leakage and retinal ischemia will be measured by UWF-FA.
Vascular density and foveal avascular zone will be measured with OCT-A (if available).
Retinal physiology will be measured by ERG (if available).

Every effort is to be made to have the same person perform the measurements at all time points and at all visits.

Safety endpoints will include:
Incidence and severity of systemic and ocular AEs.
Shift from baseline in body system assessments (cardiovascular, musculoskeletal, neurological, respiratory, skin, and other).
Change from baseline in vital sign measurements.
Change from baseline in clinical laboratory assay results (blood chemistry, hematology).
Shift from baseline in slit lamp examination parameters.
Shift from baseline in dilated funduscopic examination parameters.
Change from baseline in IOP.
Percent of subjects with a decrease of ≥5 letters in ETDRS BCVA compared to baseline.

Urine pregnancy tests for females of childbearing potential will be conducted at each study visit.

### More Detailed Procedures for Assessing Recording and Analyzing of Efficacy Parameters

Following the Day 1 Visit, site visits will occur at Week 4, Week 8, Week 12, and every 3 months for 24 months during the treatment period, for a total of 10 such visits. In between these visits, subjects will be contacted by telephone at various time points for a safety assessment to include AEs, concomitant medications, and drug compliance.

### Screening/Visit 1 (Day −28 to −1)

Subjects with moderate to severe NPDR in both eyes will be selected for study participation and be screened for study eligibility. Once a subject arrives at the study site, a member of the study staff will interview the individual as to their qualifications for participation in the study and explain the study to the subject, and if the subject wishes to continue, the informed consent form (ICF) is signed.

At the Screening Visit, a subject identification number will be assigned and medical and ophthalmic history, demographics, and information on prior/concomitant medications will be collected. HR/BP, ETDRS BCVA, color FP, SD-OCT, ERG (if available), UWF-FA, kidney function tests, and a urine pregnancy test (for females of childbearing potential) will be performed. Diabetic Retinopathy Severity Scale and CST eligibility will be determined by a central reading center, and additional ophthalmic and non-ophthalmic eligibility criteria will be evaluated.

### Baseline/Visit 2 (Day 1)

On Day 1, medical/ophthalmic history will be re-examined, and eligible subjects will be randomized into the study and take their first dose of study medication at the study site. Prior to dosing on Day 1, vital signs and blood samples will be taken for baseline clinical laboratory assessments. Early Treatment Diabetic Retinopathy Study BCVA, slit lamp biomicroscopy, dilated indirect ophthalmoscopy, IOP, SD-OCT, OCT-A (at select study sites), and ERG (at select study sites) will also be performed and the NEI-VFQ-25 and SF-12 will be completed by the subjects to obtain baseline values. For color FP and UWF-FA, results collected at the Screening Visit will serve as the baseline values.

Blood sampling for PK analysis will be conducted in approximately 200 subjects at a subset of study sites. A venous blood sample will be collected between 2 and 4 hours post-dose on Day 1.

Adverse events and concomitant medications will be assessed, females of childbearing potential will take a urine pregnancy test, and study medication and study medication diaries will be dispensed.

### Telephone Safety Call Visit 3 (Week 1±3 Days)

Subjects will be contacted by the site by telephone for a safety assessment to include:
Drug compliance.
Concomitant medications.
Adverse events.

### Treatment Visit 4 (Week 4±7 Days)

Subjects will return to the site for in-clinic dosing and a series of safety and efficacy assessments as follows:
Best-corrected VA (ETDRS).
Adverse events.

Following the completion of the assessments, new study medication and medication diaries will be dispensed.

### Treatment Visit 5 (Week 8±7 Days)

Subjects will return to the site for in-clinic dosing and a series of safety and efficacy assessments as follows:
Best-corrected VA (ETDRS).
Adverse events.

Following the completion of the assessments, new study medication and medication diaries will be dispensed.

### Treatment Visit 6 (Week 12±7 Days)

Subjects will return to the site for in-clinic dosing and a series of safety and efficacy assessments as follows:
Concomitant medications.
Urine pregnancy test (females of childbearing potential only).
Vital signs.
Best-corrected VA (ETDRS).
Color FP.
Spectral-domain OCT.
Electroretinography (if available).
Slit lamp biomicroscopy.
Dilated indirect ophthalmoscopy.
Intraocular pressure.
Pharmacokinetic sampling (prior to the morning dose and 2-4 hours post dose).
Adverse events.

Following the completion of the assessments, tablets will be counted and medication diaries will be reviewed for drug compliance and tablets returned for drug accountability. New study medication and medication diaries will be dispensed.

### Telephone Safety Call Visit 7 (Week 18±3 Days)

Subjects will be contacted by the site by telephone for a safety assessment to include:
Drug compliance.
Concomitant medications.
Adverse events.

### Treatment Visit 8 (Week 24±7 Days)

Subjects will return to the site for in-clinic dosing and a series of safety and efficacy assessments as follows:
Concomitant medications.
National Eye Institute VFQ-25.
12-item Short Form Health Survey.
Urine pregnancy test (females of childbearing potential only)
Vital signs.
Best-corrected VA (ETDRS).
Color FP.
Spectral-domain OCT.
Optical coherence tomography angiography (if available).
Electroretinography (if available).
Ultra-widefield fluorescein angiography.
Slit lamp biomicroscopy.
Dilated indirect ophthalmoscopy.
Intraocular pressure.
Blood chemistry (CMP).
Blood hematology (CBC).
Hemoglobin A1c.
Kidney function (eGFR, UACR).
Pharmacokinetic sampling (prior to the morning dose and 2-4 hours post dose; at select study sites).
Adverse events.

Following the completion of the assessments, tablets will be counted and medication diaries will be reviewed for drug compliance and tablets returned for drug accountability. New study medication and study medication diaries will be dispensed.

### Telephone Safety Call Visit 9 (Week 30±3 Days)

Subjects will be contacted by the site by telephone for a safety assessment to include:
Drug compliance.
Concomitant medications.
Adverse events.

### Treatment Visit 10 (Week 36±7 Days)

Subjects will return to the site for a series of safety and efficacy assessments as follows:
Concomitant medications.
Urine pregnancy test (females of childbearing potential only).
Vital signs.
Best-corrected VA (ETDRS).
Spectral-domain OCT.
Electroretinography (if available).
Slit lamp biomicroscopy.
Dilated indirect ophthalmoscopy.
Intraocular pressure.
Adverse events.

Following the completion of the assessments, tablets will be counted and medication diaries will be reviewed for drug compliance and tablets returned for drug accountability. New study medication and study medication diaries will be dispensed.

### Telephone Safety Call Visit 11 (Week 42±3 Days)

Subjects will be contacted by the site by telephone for a safety assessment to include:
Drug compliance.
Concomitant medications.
Adverse events.

### Treatment Visit 12 (Week 48±7 Days)

Subjects will return to the site for in-clinic dosing and a series of safety and efficacy assessments as follows:
Concomitant medications.
National Eye Institute VFQ-25.
12-item Short Form Health Survey.
Urine pregnancy test (females of childbearing potential only).
Vital signs.
Best-corrected VA (ETDRS).
Color FP.
Spectral-domain OCT.
Optical coherence tomography angiography (if available).
Electroretinography (if available).
Ultra-widefield fluorescein angiography.
Slit lamp biomicroscopy.
Dilated indirect ophthalmoscopy.
Intraocular pressure.
Blood chemistry (CMP).
Blood hematology (CBC).
Hemoglobin A1c.
Kidney function (eGFR, UACR).
Pharmacokinetic sampling (prior to the morning dose and 2-4 hours post dose).
Adverse events.

Following the completion of the assessments, tablets will be counted and medication diaries will be reviewed for drug compliance and tablets returned for drug accountability. New study medication and study medication diaries will be dispensed.

### Telephone Safety Call Visit 13 (Week 54±3 Days)

Subjects will be contacted by the site by telephone for a safety assessment to include:
Drug compliance.
Concomitant medications.
Adverse events.

### Treatment Visit 14 (Week 60±7 Days)

Subjects will return to the site for a series of safety and efficacy assessments as follows:
Concomitant medications.
Urine pregnancy test (females of childbearing potential only).
Vital signs.
Best-corrected VA (ETDRS).
Spectral-domain OCT.
Electroretinography (if available).
Slit lamp biomicroscopy.
Dilated indirect ophthalmoscopy.
Intraocular pressure.
Adverse events.

Following the completion of the assessments, tablets will be counted and medication diaries will be reviewed for drug compliance and tablets returned for drug accountability. New study medication and study medication diaries will be dispensed.

### Telephone Safety Call Visit 15 (Week 66±3 Days)

Subjects will be contacted by the site by telephone for a safety assessment to include:
Drug compliance.
Concomitant medications.
Adverse events.

### Treatment Visit 16 (Week 72±7 Days)

Subjects will return to the site for a series of safety and efficacy assessments as follows:
Concomitant medications.
National Eye Institute VFQ-25.
12-item Short Form Health Survey.
Urine pregnancy test (females of childbearing potential only).
Vital signs.
Best-corrected VA (ETDRS).
Color FP.
Spectral-domain OCT.
Optical coherence tomography angiography (if available).
Electroretinography (if available).
Ultra-widefield fluorescein angiography.
Slit lamp biomicroscopy.
Dilated indirect ophthalmoscopy.
Intraocular pressure.
Blood chemistry (CMP).
Blood hematology (CBC).
Hemoglobin A1c.
Kidney function (eGFR, UACR).
Adverse events.

Following the completion of the assessments, tablets will be counted and medication diaries will be reviewed for drug compliance and tablets returned for drug accountability. New study medication and study medication diaries will be dispensed.

### Telephone Safety Call Visit 17 (Week 78±3 Days)

Subjects will be contacted by the site by telephone for a safety assessment to include:
Drug compliance.
Concomitant medications.
Adverse events.

### Treatment Visit 18 (Week 84±7 Days)

Subjects will return to the site for a series of safety and efficacy assessments as follows:
Concomitant medications.
Urine pregnancy test (females of childbearing potential only).
Vital signs.
Best-corrected VA (ETDRS).
Spectral-domain OCT.
Electroretinography (if available).
Slit lamp biomicroscopy.
Dilated indirect ophthalmoscopy.
Intraocular pressure.
Adverse events.

Following the completion of the assessments, tablets will be counted and medication diaries will be reviewed for drug compliance and tablets returned for drug accountability. New study medication and study medication diaries will be dispensed.

### Telephone Safety Call Visit 19 (Week 90±3 Days)

Subjects will be contacted by the site by telephone for a safety assessment to include:
Drug compliance.
Concomitant medications.
Adverse events.

### End of Treatment Visit 20 (Week 96±7 Days)

Subjects will return to the site for a series of safety and efficacy assessments as follows:
Concomitant medications.
National Eye Institute VFQ-25.
12-item Short Form Health Survey.
Urine pregnancy test (females of childbearing potential only).
Vital signs.
Best-corrected VA (ETDRS).
Color FP.
Spectral-domain OCT.
Optical coherence tomography angiography (if available).
Electroretinography (if available).
Ultra-widefield fluorescein angiography.
Slit lamp biomicroscopy.
Dilated indirect ophthalmoscopy.
Intraocular pressure.
Blood chemistry (CMP).
Blood hematology (CBC).
Hemoglobin A1c.
Kidney function (eGFR, UACR).
Adverse events.

Following the completion of the assessments, tablets will be counted and medication diaries will be reviewed for drug compliance and accountability.

### Follow-Up Telephone Visit 21 (Week 97±3 Days)

Subjects will be contacted by the site by telephone for a safety assessment to include:
Concomitant medications.
Adverse events.

### Unscheduled Visits

An Unscheduled Visit may be any visit to the investigator other than the specific visits requested in the protocol. The Investigator will perform all procedures necessary to evaluate the subject at these visits and record any AEs in the CRF.

### Adverse Event Definitions

Adverse events may be characterized as follows for the clinical study:

Adverse event. An AE is any untoward medical occurrence in a patient or clinical study subject administered a study medication (pharmacological/biological product) that does not necessarily have a causal relationship to this medication. An AE can therefore be any unfavorable and unintended sign (including abnormal laboratory findings), symptom, or disease temporally associated with the use of the study medication, whether or not related to the study medication. Study medication includes the investigational drug under evaluation and the comparator product or vehicle placebo that is given or administered during any phase of the study.

Medical conditions/diseases present before starting the investigational treatment are only considered AEs if they worsen after starting the investigational treatment. Abnormal test results constitute AEs only if they induce clinical signs or symptoms, are considered clinically significant, or require therapy.

The occurrence of AEs should be sought by open-ended questioning of the subject at each visit during the study. At each clinic assessment/visit, study personnel should ask the following question: “Have you had any problems since your last visit?”. Adverse events also may be detected when they are volunteered by the subject during or between visits or through study assessments.

Life-threatening AE or life-threatening suspected adverse reaction. An AE or suspected adverse reaction is considered “life-threatening” if, in the view of the investigator, its occurrence places the subject at immediate risk of death. It does not include an AE or suspected adverse reaction that, had it occurred in a more severe form, might have caused death.

Serious AE or serious suspected adverse reaction. An AE or suspected adverse reaction is considered “serious” if, in the view of the investigator, it results in any of the following outcomes at any dose:
Death.
Life threatening.
Initial or prolonged hospitalization.
Disability or permanent damage.
Congenital anomaly or birth defect.
Needs intervention to prevent impairment.
Other medically important serious event.

Important medical events that may not result in death, be life threatening, or require hospitalization may be considered serious when, based upon appropriate medical judgment, they may jeopardize the subject, or the subject may require medical or surgical intervention to prevent one of the outcomes listed in this definition.

Treatment on an outpatient emergency basis that does not result in hospital admission, or a hospitalization that is elective or is a preplanned treatment for a pre-existing condition that is unrelated to the medication under study and has not worsened since the start of the study, is not considered an SAE.

Suspected/Related adverse reaction means any AE for which there is a reasonable possibility that the drug caused the AE. For the purposes of Investigational New Drug (IND) safety reporting, “reasonable possibility” means there is evidence to suggest a causal relationship between the drug and the AE. Suspected adverse reaction implies a lesser degree of certainty about causality than an expected adverse reaction, which means any AE caused by a drug.

Unexpected AE or unexpected suspected adverse reaction. An AE or suspected adverse reaction is considered “unexpected” if it is not listed in the Investigators' Brochure or is not listed at the specificity or severity that has been observed; or, if an Investigators' Brochure is not required or available, is not consistent with the risk information described in the general investigational plan or elsewhere in the current application, as amended. “Unexpected,” as used in this definition, also refers to AEs or suspected adverse reactions that are mentioned in the Investigators' Brochure as occurring with a class of drugs or as anticipated from the pharmacological properties of the drug but are not specifically mentioned as occurring with the particular drug under investigation.

The study medication relationship for each AE/adverse reaction should be determined by the investigator using these explanations:
Not related.
Unlikely related.
Possibly related.
Probably related.
Definitely related.
Unknown.

If there is any valid reason, even if undetermined, for suspecting a possible cause-and-effect relationship between the study medication and the occurrence of the AE, then the AE should be considered “related.”

Severity of an AE is defined as a qualitative assessment of the level of discomfort of an AE as is determined by the Investigator or reported to him/her by the subject. The assessment of severity is made irrespective of study medication relationship or seriousness of the event and should be evaluated according to the following scale:
1=Mild: Present, but not distressing, and no disruption of normal daily activity.
2=Moderate: Discomfort sufficient to reduce or affect normal daily activity.
3=Severe: Incapacitating, with inability to work or perform normal daily activity.

A change in severity for a reported AE will require an end date for the previous severity and a new start and end date for the new severity. For example, a change in severity may go from mild to severe or from severe to moderate. In either case, the start or end dates should be recorded.

The term “severe” is used to describe the intensity of an event/reaction; the event/reaction itself may be of relatively minor medical significance (such as a severe headache). This is not the same as a “serious” AE, which is based on a subject/event outcome or action criteria usually associated with events that pose a threat to the subject's life or vital functions. “Seriousness” (NOT severity) serves as a guide for defining regulatory reporting obligations.

Action taken in response to an AE is coded as:
Dose not changed: An indication that a medication schedule was maintained.
Dose reduced: An indication that a medication schedule was modified by subtraction, either by changing the frequency, strength, or amount.
Dose interrupted: An indication that a medication schedule was modified by temporarily terminating a prescribed regimen of medication.
Drug discontinued: An indication that a medication schedule was modified through termination of a prescribed regimen of medication.
Not applicable: Determination of a value is not relevant in the current context.
Unknown: Not known, not observed, not recorded, or refused.

Additional other actions taken:
Concomitant medication.
Hospitalization.

Outcome of an AE is coded as:
Fatal: The termination of life as a result of an AE.
Not recovered/Not resolved: One of the possible results of an AE outcome that indicates that the event has not improved or recuperated.
Recovered/Resolved: One of the possible results of an AE outcome that indicates that the event has improved or recuperated.
Recovered/Resolved with sequelae: One of the possible results of an AE outcome where the subject recuperated but retained pathological conditions resulting from the prior disease or injury.
Recovering/Resolving: One of the possible results of an AE outcome that indicates that the event is improving.
Unknown: Not known, not observed, not recorded, or refused.
   Statistical Analysis of Data Obtained from Study

Data obtained from the study may be processed using statistical analysis procedures described in the literature or as describe din more detail below.

### A. Sample Size

A sample size of 450 subjects who are evaluable for efficacy (i.e., who have at least 1 post-randomization DRSS evaluation and do not have any major protocol deviations considered to have significant impact on treatment outcome) is desirable for the study. The primary treatment comparison will be Active Compound versus placebo (α=0.05 significance, 2-tailed).

All subjects will be randomized into the study in a 1:1 ratio to either Active Compound or placebo.

### B. Analysis Populations

Modified Intention-to-Treat: The mITT Population will include all randomized subjects who received at least 1 dose of study treatment and at least 1 post-treatment DRSS evaluation. The mITT Population will be used for primary endpoint analysis and to analyze other efficacy endpoints, with subjects included in their randomized treatment regardless of the treatment they actually received.

Per Protocol Population: The PP Population will include all subjects in the mITT Population who have missed less than 20% of expected doses and do not have any major protocol deviations considered to have significant impact on treatment outcome. The PP Population will be used to analyze select efficacy endpoints as specified in the Statistical Analysis Plan (SAP), with subjects included in their randomized treatment regardless of the treatment they actually received.

Completer Population: The Completer Population will include all subjects in the PP Population who have at least 1 DRSS measurement at Week 96 and no rescue treatment in the study eye. The Completer Population will be used to analyze select efficacy endpoints as specified in the SAP, with subjects included in their randomized treatment regardless of the treatment they actually received.

All Randomized Population (ARP): The ARP will include all randomized subjects. This population is also known as the Intention-to-Treat (ITT) Population.

Safety Population (SP): The SP will include all randomized subjects who have received at least 1 dose of study treatment. The SP will be used to summarize safety variables, using the actual treatment a subject received.

Pharmacokinetic Population: The PK Population will include all randomized subjects who have received at least 1 dose of study treatment and have at least 1 measurable study drug concentration in plasma. The PK Population will be used for the Population PK analysis using the actual treatment a subject received.

### C. Statistical Methods—General Considerations

Descriptive summaries of variables will be provided where appropriate. For continuous variables, the number of non-missing values (n), mean, standard deviation, median, minimum, and maximum will be tabulated by treatment group. For categorical variables, the counts and proportions of each value will be tabulated by treatment group. Expansion of descriptive table categories within each treatment may occur if such elaborations are thought to be useful.

### D. Statistical Methods—Demographic and Baseline Characteristics

Demographic and baseline characteristics such as age (year), sex, race, ethnicity, study eye, and DRSS of study eye at Screening will be summarized for the ARP, SP, mITT Population, PP Population, PK Population, and Completer Population.

### E. Statistical Methods—Subject Disposition

A summary of the analysis sets includes the number and percentage of subjects by treatment group and overall for the ARP, SP, mITT Population, PP Population, and PK Population. End-of-trial information will also be summarized in this table, including the number of subjects completing the study, the number of subjects who prematurely withdrew from the study with reasons for withdrawal, the number of subjects completing the study medication dosing, and the number of subjects who prematurely discontinued the study medication with reasons for study medication discontinuation. A by-subject data listing of study completion information, including the reason for premature study withdrawal, if applicable, will be presented.

### F. Statistical Methods—Medical History and Prior/Concomitant Medications

The number and percent of subjects with individual medical, ocular, and diabetic eye histories will be summarized for all subjects by treatment group and overall. Non-ocular medical history, ocular medical history, and diabetic eye history will be summarized separately. Ocular and non-ocular medical history will be coded using the latest version of the Medical Dictionary for Regulatory Activities (MedDRA) and will be summarized by treatment group and overall using the SP.

Prior medications (medications with a start and stop date prior to the day of first exposure to any study medication) and concomitant medications (medications taken on or after the day of first exposure to study medication) will be summarized by treatment group, Anatomical Therapeutic Chemical level 4, and preferred term. The total number of concomitant medications and the number and percentages of subjects with at least 1 concomitant medication will be summarized by treatment group.

Medical history and prior and concomitant medications will also be provided in by-subject listings.

### G. Estimands for the Primary Efficacy Endpoint

The primary efficacy estimand, corresponding to the primary efficacy endpoint, is defined by the following components:
1. The population is restricted to the mITT Population.
2. The variable is the percent of subjects with a ≥3-step worsening from baseline on the DRSS person-level scale at Week 48.
3. The intercurrent event of treatment discontinuation or initiation of rescue treatment for DR progression in either eye. Any observations taken after initiation of rescue treatment for PDR will be excluded from analysis.
4. The population-level summary is the difference in percent of subjects with a ≥3-step worsening from baseline on the DRSS person-level scale at Week 48, analyzed using a logistic regression model with fixed effects for treatment, baseline level of disease severity in the worse eye (DRSS level 47 or 53), and baseline DRSS person-level scale as a continuous covariate.
5. Treatment: There will be 1 active treatment group (Active Compound) and 1 control group (placebo).

### H. Analysis of Efficacy

Efficacy will be assessed using the mITT, PP, and Completer Populations, with subjects included in the treatment group to which they were randomized. For the analysis of the primary efficacy endpoint, appropriate imputation techniques will be performed for missing observations or for eyes requiring rescue treatment, if applicable. If the analysis using the mITT Population shows a positive effect for Active Compound at the 0.05 level of significance, the primary endpoint will be considered met. Sensitivity analyses will also be performed using alternative assumptions on the missing data, with imputation performed as described in the SAP.

The primary efficacy endpoint will be analyzed using a logistic regression model with treatment and baseline level of disease severity in the worse eye (DRSS level 47 or 53) as factors and the baseline DRSS person-level scale as a covariate. The percent of subjects in each treatment group meeting the criteria, the odds ratio (OR) with 95% confidence interval (CI), and p-value will be provided.

Each of the continuous secondary efficacy endpoints will be analyzed using analysis of covariance (ANCOVA) with change from baseline as the dependent variable, treatment and baseline level of disease severity in the worse eye (DRSS level 47 or 53) as factors, and the respective baseline value included as the covariate. The output from each ANCOVA will include the least-squares mean (LSM) and standard error for both treatment groups, along with the placebo-corrected LSM, its 95% CI, and associated p-value.

For each of the secondary endpoints related to percent of subjects achieving certain criteria, the analysis will be performed using a logistic regression model with treatment and baseline level of disease severity in the worse eye (DRSS level 47 or 53) as factors, and the respective baseline as a covariate. For each analysis, the percentage of subjects in each treatment group meeting the criteria, the OR with 95% CI and p-value will be provided.

For each of the secondary endpoints related to the time to an event, the analysis will be performed using a Cox proportional hazards regression model with treatment and baseline level of disease severity in the worse eye (DRSS level 47 or 53) as factors, and the respective baseline as a covariate. For each analysis, the hazard ratio with 95% CI and p-value will be provided.

If there are sufficient missing data for select secondary endpoints, appropriate multiple imputation methods will be applied as sensitivity analyses. The endpoints that will be imputed for missing data will be specified in the SAP.

Relationships between Active Compound concentrations and the change from baseline in DRSS will be explored using scatter plots or other displays. Scatter plots will include a simple linear regression line and a correlation value.

In order to adjust for multiple comparisons, select efficacy endpoints will be included in a hierarchical analysis, which will be specified in the SAP. The first endpoint in the hierarchy will be the primary efficacy endpoint, followed by key secondary endpoints.

### I. Analysis of Safety

Safety will be assessed using the SP, with subjects included in the treatment group they actually received, regardless of their randomized treatment. Observed case data will be used; no imputation will be performed for missing safety data except for the limited situations described in the SAP.

Continuous safety endpoints collected at each visit will be summarized with number of subjects, mean, standard deviation, median, minimum, and maximum for each treatment group and for all subjects. Qualitative variables collected at each visit will be summarized using counts and percentages for each treatment group and for all subjects. Summaries will also include change from baseline and shift tables, where appropriate. Individual subject safety data will be listed.

Verbatim descriptions of AEs will be coded using the latest version of MedDRA. Only TEAEs (those that occur after the first dose of study medication or increasing in severity after initiation of study medication) will be summarized in the tables and listings.

The following summary tables will be presented for TEAE data, by system organ class (SOC) and preferred term:
Treatment-emergent AEs.
Serious TEAEs.
Treatment-emergent AEs leading to withdrawal from the study.
Treatment-emergent AEs leading to study medication discontinuation.
Treatment-emergent AEs by maximum severity (mild, moderate, severe).
Treatment-emergent AEs by greatest relationship (not related, unlikely unrelated, possibly related, probably related, definitely related, or unknown).
Treatment-related severe TEAEs.
Treatment-related serious TEAEs.
Treatment-related TEAEs leading to withdrawal from the study.
Treatment-related TEAEs leading to study medication discontinuation.

### Definitions Used in Clinical Study Description

The following definitions apply for this clinical study description:

| Abbreviation | Full term |
|---|---|
| AE | adverse event |
| ALT | alanine aminotransferase |
| ANCOVA | analysis of covariance |
| APE1/Ref-1 | apurinic/apyrimidinic endonuclease |
| or Ref-1 | 1/redox factor-1 |
| ARP | All Randomized Population |
| ASNV | anterior-segment neovascularization |
| AST | aspartate aminotransferase |
| BCVA | best-corrected visual acuity |
| BID | twice daily |
| BP | blood pressure |
| CBC | complete blood count |
| CI | confidence interval |
| CI-DME | center-involved diabetic macular |
| | edema |
| CRF | case report form |
| CST | central subfield thickness |
| DMC | Data Monitoring Committee |
| DME | diabetic macular edema |
| DR | diabetic retinopathy |
| DRSS | Diabetic Retinopathy Severity Scale |
| EDC | electronic data capture |
| eGFR | estimated glomerular filtration rate |
| EOT | End of Treatment |
| ERG | electroretinography |
| ETDRS | Early Treatment Diabetic Retinopathy |
| | Study |
| FDA | Food and Drug Administration |
| FP | fundus photography |
| GCP | Good Clinical Practice |
| GLP-1 | glucagon-like peptide 1 |
| HbA1c | hemoglobin A1c |
| HIF-1α | hypoxia-inducible factor 1-alpha |
| HR | heart rate |
| HRQOL | health-related quality of life |
| ICH | International Council for Harmonisation |
| IND | Investigational New Drug |
| IOP | intraocular pressure |
| IRB | Institutional Review Board |
| ITT | Intention-to-Treat |
| LSM | least-squares mean |
| MDRD | Modification of Diet in Renal Disease |
| MedDRA | Medical Dictionary for Regulatory |
| | Activities |
| mITT | Modified Intention-to-Treat |
| NEI-VFQ-25 | 25-Item National Eye Institute Visual |
| | Function Questionnaire |
| NF-κB | nuclear factor kappa B |
| NPDR | non-proliferative diabetic retinopathy |
| NVG | neovascular glaucoma |
| OCT | optical coherence tomography |
| OCT-A | optical coherence tomography angiography |
| OR | odds ratio |
| PDR | proliferative diabetic retinopathy |
| PK | pharmacokinetic |
| PO | orally |
| PP | Per Protocol |
| PRP | panretinal laser photocoagulation |
| redox | reduction-oxidation |
| SAE | serious adverse event |
| SAP | Statistical Analysis Plan |
| SD-OCT | spectral-domain optical coherence tomography |
| SF-12 | Medical Outcomes Study 12-Item Short Form |
| | Health Survey |
| SGLT-2 | sodium-glucose transport protein 2 |
| SOC | system organ class |
| SP | Safety Population |
| TEAE | treatment-emergent adverse event |
| UACR | urine albumin-creatinine ratio |
| US | United States |
| UWF-FA | ultra-widefield fluorescein angiography |
| VA | visual acuity |
| VEGF | vascular endothelial growth factor |
| YAG | yttrium aluminum garnet |

### Example 3—Preparation of Calcium Salt of Compound 1

About 20 g of Compound 1 (1 eq.) was weighed and added to a 1 L glass reactor and mixed at room temperature with H₂O:MeOH (1:1 v/v) to provide a fine suspension having a concentration of 36.3 g of Compound 1/L of H₂O:MeOH (1:1 v/v). Compound 1 has the formula:

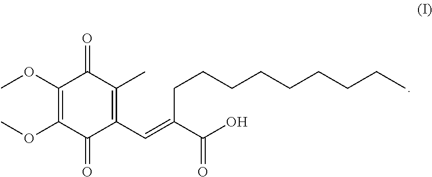 [SMILES: CCCCCCCCC/C(=C\C1=C(C)C(=O)C(OC)=C(OC)C1=O)C(=O)O]

The suspension was allowed to stir for 30 minutes with a mechanical stirrer type OS-20 with a rod and PTFE propeller at 300 rpm. Calcium hydroxide (95%; 2.0953 g; 0.5 eq.) was added, and the resultant mixture was allowed to stir at 500 rpm at room temperature for 2 hours. After 2 hours, the resultant precipitate was vacuum filtered, washed with 400 mL of H₂O:MeOH (1:1 v/v). The washed precipitate was dried in a fume hood at atmospheric pressure at 25° C. for about 45 hours to provide a Compound 1 calcium salt as a light orange solid (89.06% yield).

Purity of the Compound 1 calcium salt was determined by HPLC chromatography using an Agilent 1260 Infinity HPLC device, at 30° C. and a HiChrom C18 column (4.6×100 mm, 3.5 μm). The HPLC device was coupled with a UV-Vis Diode Array Detector (HPLC-DAD). Analyses were run with a gradient method (as described in Table below) using 0.1% formic acid in purified MilliQ water (mobile phase A) and 0.1% formic acid in acetonitrile HPLC grade (mobile phase B). A flow rate was 1 mL/min and an injection volume was 5 μL. UV detection was run at 236 nm and 266 nm. The sample was dissolved into MilliQ pure water. Purity of Compound 1 calcium salt determined by HPLC at 236 nm was 99.7% and at 266 nm was 99.8%.

| Time | % (v/v) | % (v/v) |
|---|---|---|
| (minutes) | Mobile Phase A | Mobile Phase B |
| 0.0 | 50 | 50 |
| 3.0 | 50 | 50 |
| 10.0 | 10 | 90 |
| 13.0 | 10 | 90 |
| 15.0 | 50 | 50 |

The Compound 1 calcium salt was analyzed by XRPD, and thermo-gravimetric (TG)/differential scanning calorimetric (DSC).

FIG. 4A shows an XRPD diffractogram (background subtracted) of the Compound 1 calcium salt obtained as described in this Example, and Table 1 lists XRPD peaks represented in FIG. 4A.

TABLE 1
XRPD Data for Compound 1 calcium salt from this Example

| 2-theta | d Value | Net Intensity | Rel. |
|---|---|---|---|
| Angle (°) | (Å) | (Counts) | Intensity (%) |
| 4.12 | 21.46 | 563.62 | 95.22 |
| 5.35 | 16.50 | 591.88 | 100.00 |
| 5.76 | 15.33 | 162.75 | 27.50 |
| 6.37 | 13.86 | 218.93 | 36.99 |
| 8.29 | 10.66 | 34.36 | 5.81 |
| 9.23 | 9.57 | 21.57 | 3.64 |
| 9.63 | 9.18 | 37.84 | 6.39 |
| 10.80 | 8.19 | 56.31 | 9.51 |
| 11.44 | 7.73 | 73.04 | 12.34 |
| 11.99 | 7.38 | 86.61 | 14.63 |
| 12.31 | 7.19 | 123.50 | 20.87 |
| 12.81 | 6.90 | 76.07 | 12.85 |
| 13.12 | 6.74 | 44.24 | 7.47 |
| 13.77 | 6.43 | 138.15 | 23.34 |
| 14.16 | 6.25 | 110.33 | 18.64 |
| 16.49 | 5.37 | 26.36 | 4.45 |
| 16.57 | 5.35 | 33.45 | 5.65 |
| 17.16 | 5.16 | 37.73 | 6.37 |
| 17.61 | 5.03 | 50.16 | 8.47 |
| 20.35 | 4.36 | 39.70 | 6.71 |
| 20.92 | 4.24 | 57.83 | 9.77 |
| 21.89 | 4.06 | 39.48 | 6.67 |
| 23.71 | 3.75 | 44.47 | 7.51 |
| 26.33 | 3.38 | 29.20 | 4.93 |

TG/DSC analysis: A sample of the Compound 1 calcium salt (about 5.3 mg) obtained according to this Example was weighed into an open aluminum pan, loaded into a simultaneous Setaram LABSYS EVO thermo-gravimetric/differential scanning calorimeter (TG-DTA/DSC) and maintained at 30° C. for 15 minutes. The sample was then heated from 30° C. to 550° C., during which time a change in sample weight was recorded along with any differential thermal events. Nitrogen was used as a purge gas, at a flow rate of 180 cm³/min. Prior to the analysis, the instrument mass loss and temperature were calibrated using copper sulfate pentahydrate and reference standards (lead and indium), respectively. The sample analysis was performed using CALISTO software, where the corresponding mass loss and temperatures of thermal events were quoted as the onset temperature, measured according to the manufacturer's specifications. The analysis was carried out with a heating rate of 10° C./minute and the background was subtracted before further processing.

The TG/DSC analysis of the Compound 1 calcium salt obtained according to this Example showed one small endothermic event between 50° C. and 89° C. (peak maximum at about 82° C.) and one exothermic event starting at about 208° C. (peak maximum at about 213° C.) (FIG. 4B). A mass loss of about 5% was observed on the TG curve, corresponding to the endothermic event. Based on the TG/DSC data, the Compound 1 calcium salt obtained according to this Example is a monohydrate.

Compound 1 calcium salt obtained generally according to this method was further analyzed by FT-IR, FT-Raman, dynamic vapor sorption (DVS), and ¹H NMR.

An FT-IR spectrum of the Compound 1 calcium salt was recorded at between 3500 and 50 cm⁻¹ showing the following significant bands, expressed in wavenumbers (cm⁻¹): 2924 (m) and 2853 (m) specific to asymmetric —CH₂—, symmetric —CH₃ and —CH₂— stretching vibrations; 1636 (st) characteristic to —C═O stretching and some ═C—H stretching vibrations; 1603 (st) specific to —C═C— stretching and —C—C— skeletal vibrations; 1556 (st) also specific to —C—O— vibrations (e.g. in the —COOR groups) or aromatic —C═C—stretching vibrations; 1418 (st) characteristic for —C—C— stretch (in-ring) aromatics or —C—H bending vibrations; 1298 (m), 1261 (st), 1203 (st) and 1157 (m) specific to —C—O or —C(O)—O stretching vibrations; 1094 (m), 997 (m), 951 (w), 787 (w), 739 (m) and 636 (w) characteristic for ═C—H out-of-plane bending or —C—C— bending vibrations. The intensities of the absorption bands are indicated as follows: (w)=weak; (m)=medium; and (st)=strong intensities.

An FT-Raman spectrum of the Compound 1 calcium salt was recorded at between 4000 and 150 cm⁻¹ showing the following significant bands, expressed in wavenumbers (cm-1): 2927 (m) and 2858 (m) assigned to —CH₂ and —CH₃ vibrations; 1652 (st) assigned to —C═O group, 1600 (m) attributed to —C═C— vibrations; 1440 (m), 1384 (m), 1337 (st) and 1303 (m) attributed to ring deformation and —C—O— vibrations; 957 (w), 874 (w), 499 (m), 443 (m) and 344 (w) assigned to —C—C— vibrations and ring breathing. The intensities of the absorption bands are indicated as follows: (w)=weak; (m)=medium; and (st)=strong intensities. The error margin for all absorption bands of FT-Raman is ±2 cm⁻¹.

DVS Analysis: Compound 1 calcium salt had a water content of 0.39% and was equilibrated to 0 before starting the analysis. DVS analysis of the Compound 1 calcium salt (FIG. 4C) showed that the maximum water adsorption at 82% RH was about 1.52%, while at the end of the desorption branch the Compound 1 calcium salt retained 0.19% water. The pattern of a subsequent XRPD analysis revealed the same peaks, although with slightly lower intensities, and supports that post-DVS sample of the Compound 1 calcium salt maintained the same crystalline form as the pre-DVS sample of the Compound 1 calcium salt.

A ¹H-NMR spectrum of the Compound 1 calcium salt was obtained and no —COOH group peak (˜12 ppm) was observed. 6 ¹H (600 MHz, DMSO-d₆): 0.84 (3H), 1.12 (6H), 1.18 (4H), 1.23 (2H), 1.33 (2H), 1.83 (3H), 1.98 (2H), 3.88 (3H), 3.91 (3H), 6.85 (1H).

### Example 4—Preparation of Calcium Salt of Compound 1

About 75 g (0.198 mol) of Compound 1 (1 eq.) was charged to a 5 L jacketed reactor. 2025 mL (27.0 vol) of 2:1 H₂O:MeOH (v/v) was charged to the 5 L jacketed reactor at room temperature. Compound 1 has the formula:

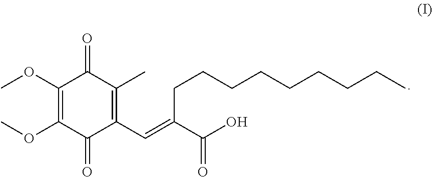 [SMILES: CCCCCCCCC/C(=C\C1=C(C)C(=O)C(OC)=C(OC)C1=O)C(=O)O]

The reaction mixture was agitated for 25 minutes at 25±5° C. to provide a fine suspension. Calcium hydroxide (7.3 g, 0.5 eq.) was charged to the reactor portion-wise over 25 minutes. The resultant mixture was thick but remained stirrable with some shelling observed near the top of the reactor. The shelled material was scraped down. The mixture was agitated at 25±5° C. for 2 hours and the mixture became very thick. A sample was taken to confirm formation of Compound 1 calcium salt by an XRPD analysis.

The resultant solids were collected via vacuum filtration and were washed twice with 2:1 water/methanol (900 mL, 12.0 vol). The solids were dried under vacuum until they had a water content of about 12% and dried using humidified drying. The humidified drying was performed using a vacuum oven attached to a house vacuum system with a small bleed of nitrogen that was saturated with water vapor. The water vapor-saturated nitrogen was obtained by bubbling nitrogen through water and had 100% humidity at 20±5° C. Humidified drying continued until the solids had a water content of 4-6% (Table 2) to provide Compound 1 calcium salt as an orange solid (80.41 g, 97.2% yield). Purity determined by HPLC at 262 nm was 99.9%. HPLC parameters and conditions are:
Mobile Phase A: 0.1% TFA in Water
Mobile Phase B: 0.1% TFA in Acetonitrile
Diluent: Acetonitrile and Water; 80:20 v/v
Sample Concentration: 0.7 mg/mL

TABLE 1

| Column: | Manufacturer: | GL Sciences Inc |
|---|---|---|
| | Name: | Inertsil ODS-2 |
| | Dimensions (mm): | 4.6 × 150 |
| | Particle Size (μm): | 5 |
| | Part Number: | 5020-01124 |
| Pump | Flow (mL/min): | 1.0 |
| | Run Time (min): | 40 |
| | Post time | Off |
| | (min): | |

| | Time | % Mobile | % Mobile |
|---|---|---|---|
| | (min) | Phase A | Phase B |
| Gradient: | 0 | 50 | 50 |
| | 5 | 50 | 50 |
| | 15 | 10 | 90 |
| | 30 | 10 | 90 |
| | 32 | 50 | 50 |
| | 40 | 50 | 50 |

| | | | | Reference | Reference |
|---|---|---|---|---|---|
| | | Wavelength | Bandwidth | Wavelength | Bandwidth |
| | Detector Type | (nm) | (nm) | (nm) | (nm) |
| Detector: | Variable | 262 | N/A | N/A | N/A |
| | Wavelength | | | | |
| | (VWD) | | | | |
| | Diode Array | 262 | 4 | Off | Off |
| | (DAD) | | | | |

| | |
|---|---|
| Peak Width | >0.025 |
| (min): | |

| | | |
|---|---|---|
| Column | Temperature (° C.): | 30 |
| Compartment: | | |
| Autosampler: | Injection Volume (μL): | 5 |
| | Temperature (° C.): | Off |
| | Needle Rinse: | Diluent, Flush |
| | | Port 3 seconds |

The Compound 1 calcium salt was analyzed by XRPD, TGA, DSC, and ¹H NMR (FIGS. 5A-5D). XRPD data were obtained using a Bruker AXS with D2 Phaser 2^{nd} Gen configuration (Part Number: A26X1-A2B0B1C) in reflection mode (scan type: Coupled TwoTheta/Theta) scanning the samples at between 3 and 40° 2-theta angles, and using the following measurements characteristics: increment per step was 0.02°, time per step was 0.3 s, and generator voltage/generator amperage was 10 mA/30 kV to reach 0.3 kW power, detector type LynxEye A17-B60 and a goniometer type Theta/Theta. The XRPD data were collected using DIFFRAC.MIEASUIRMENT 8.6.3.0 software and processed with DIFFRAC.EVA 6.0.0.8 software.

TABLE 2
Vacuum Drying Time and Water Content

| Total | Hours dried with | Water |
|---|---|---|
| hours dried | humidified drying | Content by KF |
| 0 | 0 | 59.10% |
| 4 | 0 | 62.17% |
| 6 | 0 | 55.89% |
| 8 | 0 | 57.95% |
| 10 | 0 | 56.52% |
| 27 | 0 | 16.84% |
| 31 | 0 | 12.14% |
| 34* | 0 | 33.30%* |
| 34* | 0 | 21.63%* |
| 51 | 17 | 7.01% |
| 58 | 24 | 5.67% |

*Inconsistency with this time point due to solids precipitating in the system and sticking to the detector

FIG. 5A shows an XRPD diffractogram of the Compound 1 calcium salt obtained by this Example, and Table 3 lists XRPD peaks represented in FIG. 5A.

TABLE 3
XRPD Data for Compound 1 Calcium Salt

| | | Net | |
|---|---|---|---|
| 2-theta | d Value | Intensity | Rel. |
| Angle (°) | (Å) | (Counts) | Intensity (%) |
| 5.060 | 17.45193 | 3343.52 | 100.0% |
| 5.269 | 16.75757 | 2233.80 | 66.8% |
| 7.947 | 11.11557 | 510.340 | 15.3% |
| 8.326 | 10.61072 | 100.391 | 3.0% |
| 10.085 | 8.76359 | 950.469 | 28.4% |
| 10.591 | 8.34620 | 146.969 | 4.4% |
| 11.481 | 7.70099 | 142.132 | 4.3% |
| 12.262 | 7.21232 | 259.658 | 7.8% |
| 12.777 | 6.92287 | 475.224 | 14.2% |
| 13.302 | 6.65079 | 437.195 | 13.1% |
| 14.610 | 6.05806 | 374.263 | 11.2% |
| 15.173 | 5.83474 | 152.680 | 4.6% |
| 17.173 | 5.15931 | 152.009 | 4.5% |
| 17.901 | 4.95104 | 425.727 | 12.7% |
| 18.536 | 4.78292 | 375.464 | 11.2% |
| 19.563 | 4.53410 | 464.238 | 13.9% |
| 20.547 | 4.31902 | 139.854 | 4.2% |
| 22.730 | 3.90898 | 249.891 | 7.5% |
| 23.764 | 3.74119 | 69.3565 | 2.1% |
| 24.555 | 3.62242 | 55.5633 | 1.7% |
| 25.704 | 3.46305 | 87.4043 | 2.6% |
| 27.127 | 3.28460 | 161.520 | 4.8% |

TGA analysis: A sample of the Compound 1 calcium salt (about 2-5 mg) obtained generally according to this Example, was weighed into an aluminum pan (70 μL), loaded into Mettler Toledo TGA 2 Star System (sampling interval: 1 s). The sample was then heated from 25° C. to 350° C. (heating rate of 10 K/min), during which time a change in sample weight was recorded. The sample analysis was performed using STAR 16.20 software.

DSC analysis: A sample of the Compound 1 calcium salt (about 3-6 mg) obtained generally according to this Example, was weighed into an aluminum pan (40 μL), loaded into Mettler Toledo DSC 1 Star System (sampling interval: 1 s). The sample was then heated from 25° C. to 350° C. (heating rate of 10° C./min), during which any differential thermal events were recorded. Nitrogen was used as a purge gas, at a flow rate of 80 cm³/min. The sample analysis was performed using STAR 16.10 software.

¹H NMR analysis: ¹H NMR spectrum was obtained on Bruker Advance III HD 300 MHz NMR Spectrometer (sample size: about 8 mg/mL in DMSO-d₆) and analyzed with TopSpin 3.6.4 software.

### Example 5—Preparation of Calcium Salt of Compound 1 (Predominantly Amorphous)

Compound 1 calcium salt as prepared generally according to Example 3 was heated at about 60° C. for 1 h at atmospheric pressure. The resultant Compound 1 calcium salt was determined to be anhydrous and predominantly (>50%) amorphous, as evidenced by the presence of only a few XRPD peaks. Compound 1 has the formula:

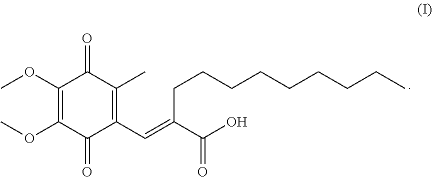 [SMILES: CCCCCCCCC/C(=C\C1=C(C)C(=O)C(OC)=C(OC)C1=O)C(=O)O]

X-ray powder diffraction (XRPD) analysis was carried out using a Bruker D8 Discover diffractometer with DAVINCI configuration, in transmission mode (scan type: TwoTheta or Offset Coupled TwoTheta/Theta) scanning the samples (˜2-3 mg) at between 1.5 and 45° 2-theta angles, and using the following measurements characteristics: acquisition time was 7.58 minutes, increment per step was 0.01°, time per step was 0.1 s, and generator voltage/generator amperage was 40 mA/40 kV to reach 1.6 kW power. The XRPD system was used in Parallel Beam Geometry (Göbel mirror) with an anode of Cu and a detector type LynxEye. Also, the XRPD system used a goniometer type Theta/Theta with a measuring circle diameter of 560 mm and vertical operating position. For transmission mode, 1 UBC collimator magnetic holder 1 mm was mounted to primary optics.

The raw XRPD data were imported in the Diffrac.EVA5.0 software and processed using the subsequent parameters: background subtraction and Kα2 stripping were performed before peak determination, and the peak search operation was performed with a threshold of 0.78 and a peak width of 0.242 (or 0.153). All resulting peaks with relative intensity greater than or equal to 2% were considered.

FIG. 6A shows an XRPD diffractogram (background subtracted) of the anhydrous and predominantly amorphous Compound 1 calcium salt obtained when heated at about 60° C. for 1 h at atmospheric pressure, and Table 1 lists XRPD peaks represented in FIG. 6A.

TABLE 1

| 2-theta | d Value | Net Intensity | Rel. |
|---|---|---|---|
| Angle (°) | (Å) | (Counts) | Intensity (%) |
| 4.45 | 19.85 | 719.68 | 100.00 |
| 6.00 | 14.72 | 60.49 | 8.40 |
| 9.11 | 9.70 | 27.73 | 3.85 |

The TG/DSC analysis of the predominantly amorphous Compound 1 calcium salt showed only one exothermic event starting at about 210° C. that was attributed to the degradation process of the salt (FIG. 6B). No mass loss was attributed to the thermal event. Based on the TG/DSC data, the predominantly amorphous Compound 1 calcium salt is anhydrous.

Hydration Experiment: Water (1.60 mL) was added to the Compound 1 calcium salt (anhydrous and predominantly amorphous) as obtained above (about 200 mg). The resultant mixture was allowed to stir at 700 rpm for 1 h. The resultant mixture was heated at about 40° C. for 2 h at atmospheric pressure then at 50° C. for 4 h at atmospheric pressure. The resultant solids showed an XRPD pattern and a TG/DSC consistent with Compound 1 calcium salt monohydrate obtained generally according to Example 3.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of preventing the progression of a diabetic retinal disease, comprising the steps of:
(a) identifying a human patient with diabetic retinal disease that would benefit from therapy that prevents the progression of the diabetic retinal disease; and
(b) orally administering to said human patient in need thereof a therapeutically effective amount of a first therapeutic agent to thereby prevent the progression of diabetic retinal disease, wherein the first therapeutic agent is a compound of Formula I or a pharmaceutically acceptable salt thereof.

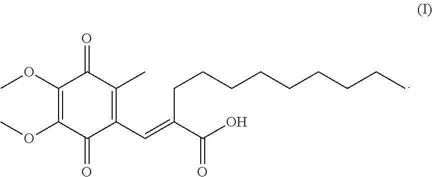 [SMILES: CCCCCCCCC/C(=C\C1=C(C)C(=O)C(OC)=C(OC)C1=O)C(=O)O]

2. The method of claim 1, wherein the diabetic retinal disease is diabetic retinopathy.

3. The method of claim 2, wherein step (a) comprises identifying a human patient with diabetic retinal disease that is mild or moderate non-proliferative diabetic retinopathy that would benefit from therapy that prevents progression of the diabetic retinal disease.

4. The method of claim 2, wherein step (a) comprises identifying a human patient with diabetic retinal disease that is moderate to moderately severe non-proliferative diabetic retinopathy that would benefit from therapy that prevents progression of the diabetic retinal disease.

5. (canceled)

6. (canceled)

7. The method of claim 3, wherein preventing the progression of diabetic retinal disease comprises preventing an increase of three or more steps in the patient's Diabetic Retinopathy Severity Score.

8. (canceled)

9. (canceled)

10. (canceled)

11. (canceled)

12. (canceled)

13. (canceled)

14. The method of claim 2, wherein the diabetic retinopathy is moderate diabetic retinopathy.

15. The method of claim 2, wherein the diabetic retinopathy is moderately severe to severe diabetic retinopathy.

16. The method of claim 2, wherein the diabetic retinopathy is non-proliferative diabetic retinopathy.

17. (canceled)

18. (canceled)

19. The method of claim 3, wherein the first therapeutic agent is a calcium salt of compound of Formula I.

20. (canceled)

21. (canceled)

22. (canceled)

23. (canceled)

24. (canceled)

25. (canceled)

26. (canceled)

27. (canceled)

28. The method of claim 3, wherein a first dose of the first therapeutic agent and a second dose of the first therapeutic agent are orally administered to the patient on the same day.

29. The method of claim 3, wherein the first therapeutic agent is orally administered to the patient in an amount of about 600 mg per day.

30. The method of claim 3, wherein about 300 mg of the first therapeutic agent is orally administered to the patient in the morning, and about 300 mg of the first therapeutic agent is orally administered to the patient in the evening.

31. The method of claim 3, wherein about 300 mg of the first therapeutic agent is orally administered to the patient, and then at a time that is from about 8 hours to about 16 hours later about 300 mg of the first therapeutic agent is orally administered to the patient.

32. (canceled)

33. (canceled)

34. (canceled)

35. (canceled)

36. (canceled)

37. (canceled)

38. The method of claim 3, wherein the first therapeutic agent is orally administered to a patient only 1 time per day.

39. (canceled)

40. The method of claim 3, wherein the first therapeutic agent is orally administered to the patient daily for at least 12 weeks.

41. (canceled)

42. (canceled)

43. The method of claim 3, wherein the first therapeutic agent is orally administered to the patient daily for at least 48 weeks.

44-65. (canceled)

66. The method of claim 3, wherein the patient has a DRSS level of 47 or 53 in at least one eye.

67. The method of claim 2, wherein the diabetic retinopathy is proliferative diabetic retinopathy or diabetic macular edema.

68. A method of reducing the rate of progression of a diabetic retinal disease, comprising the steps of:
(a) identifying a human patient with diabetic retinal disease that would benefit from therapy that reduces the rate of progression of the diabetic retinal disease; and
(b) orally administering to said human patient in need thereof a therapeutically effective amount of a first therapeutic agent to thereby reduce the rate of progression of the diabetic retinal disease, wherein the first therapeutic agent is a compound of Formula I or a pharmaceutically acceptable salt thereof:

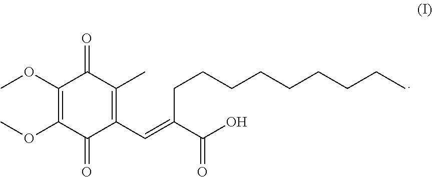 [SMILES: CCCCCCCCC/C(=C\C1=C(C)C(=O)C(OC)=C(OC)C1=O)C(=O)O]

69. A method of slowing clinically meaningful progression of a diabetic retinal disease, comprising the steps of:
(a) identifying a human patient with diabetic retinal disease that would benefit from therapy that slows clinically meaningful progression of the diabetic retinal disease; and
(b) orally administering to said human patient in need thereof a therapeutically effective amount of a first therapeutic agent to thereby slow clinically meaningful progression of the diabetic retinal disease, wherein the first therapeutic agent is a compound of Formula I or a pharmaceutically acceptable salt thereof:

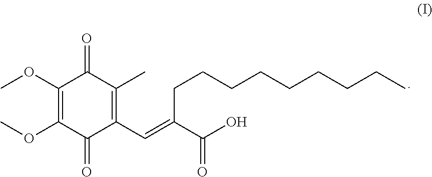 [SMILES: CCCCCCCCC/C(=C\C1=C(C)C(=O)C(OC)=C(OC)C1=O)C(=O)O]

70-115. (canceled)
